(19) United States
(12) Patent Application Publication
(10) Pub. No.: US 2026/0048083 A1
(43) Pub. Date: Feb. 19, 2026

(54) METHOD OF TREATING ALZHEIMER'S DISEASE WITH EXPANDED NATURAL KILLER CELLS

(71) Applicant: NKMAX CO., LTD., Gyeonggi-do (KR)

(72) Inventors: Paul Y. Song, Santa Monica, CA (US); Sang Woo Park, Gyeonggi-do (KR); Yong Man Kim, Gyeonggi-do (KR)

(21) Appl. No.: 19/101,563

(22) PCT Filed: Jul. 11, 2023
(86) PCT No.: PCT/US2023/070005

§ 371 Date: Feb. 5, 2025

Related U.S. Application Data
(60) Provisional application No. 63/396,865, filed on Aug. 10, 2022.
(60) Provisional application No. 63/376,050, filed on Sep. 16, 2022.
(60) Provisional application No. 63/501,962, filed on May 12, 2023.

(51) Int. Cl. A61K 35/17; A61K 40/15; A61K 45/06; A61P 25/28; C12N 5/00; C12N 5/0783; G01N 33/68

(52) U.S. Cl. CPC A61K 35/17; A61K 40/15; A61K 45/06; A61P 25/28; C12N 5/0018; C12N 5/0646; G01N 33/6896; A61K 2239/31; A61K 2239/38; C12N 2501/2302; C12N 2501/2321; C12N 2502/1114; G01N 2333/4709; G01N 2800/2821; G01N 2800/52

(57) ABSTRACT

A method for treating Alzheimer's disease is disclosed. The method comprises identifying a subject and treating the subject with expanded natural killer cells (NKs). A composition for treating Alzheimer's disease is also disclosed.

### INCORPORATION BY REFERENCE

Any and all applications for which a foreign or domestic priority claim is identified in the Application Data Sheet as filed with the present application are hereby incorporated by reference under 37 CFR 1.57.

### FIELD

The present disclosure relates to a method for treating Alzheimer's disease with natural killer cells.

### BACKGROUND

Natural killer (NK) cells have proven to be promising candidates for use in adoptive cell therapy (ACT) due to their high cytotoxicity and lower risk than T-cells. One general approach to NK ACT has been the administration of autologous NK cells expanded ex vivo.

Alzheimer's disease is a neurodegenerative disease affecting parts of the brain that control thought, memory, language, and motor system. Current treatments for Alzheimer's are associated with alleviation of cognitive and behavioral symptoms.

### SUMMARY

This application is related to methods of producing high-purity natural killer cells, and cell therapeutic compositions for treating Alzheimer's disease comprising high-purity natural killer cells and cytokines. Any features, structures, or steps disclosed herein can be replaced with or combined with any other features, structures, or steps disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

In some embodiments, methods of treating Alzheimer's disease in a subject are provided. In some embodiments, the method comprises identifying a subject, wherein the subject has Alzheimer's disease; and administering to the subject a therapeutically effective amount of an autologous natural killer cell (NK) cell population.

In some embodiments, a method of treating Alzheimer's disease in a subject is provided. In some embodiments, the method comprises: identifying a subject, wherein the subject has Alzheimer's disease; and administering to the subject an expanded NK cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a method of cell therapy is provided, comprising: identifying a subject, wherein the subject has Alzheimer's disease; and administering to the subject an expanded NK cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a population of expanded NK cells is provided. In some embodiments, the NK cells were expanded by a method that comprises: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21. In some embodiments, the population of expanded NK cells has been administered to a subject who has Alzheimer's disease.

In some embodiments, the amount of expanded NK cells administered to a subject is a therapeutically effective amount.

In some embodiments, the therapeutically effective amount of expanded NK cells comprises 0.1×10⁹ to 9×10⁹ cells.

In some embodiments, at least 2, 3, 4, 5, 6, 7, 8, 9, 10, 11, 12, 13, 14, 15, 16, 17, 18, 19, 20 or more doses of expanded NK cells is administered to the subject.

In some embodiments, IL-2 is added at a concentration of 50-1000 IU/mL during step ii).

In some embodiments, IL-21 is added at a concentration of 10-100 ng/mL during step ii).

In some embodiments, the Mini Mental State Exam (MMSE) score of the subject is between 24-30, 19-23, or 10-18 after treatment with expanded NK cells. In some embodiments, the amount of NK cells administered is sufficient to achieve said score.

In some embodiments, the MMSE score of the subject is ≥24 after treatment with expanded NK cells. In some embodiments, the amount of NK cells administered is sufficient to achieve said score.

In some embodiments, the MMSE score of the subject is ≥19 after treatment with expanded NK cells. In some embodiments, the amount of NK cells administered is sufficient to achieve said score.

In some embodiments, the MMSE score of the subject is ≥10 after treatment with expanded NK cells. In some embodiments, the amount of NK cells administered is sufficient to achieve said score.

In some embodiments, expansion of NK cells further comprises: co-culturing the at least one of CD56+ cells and/or CD3−/CD56+ cells with the combination of feeder cells, in the presence of IL-2 for a first period; and co-culturing the at least one of CD56+ cells and/or CD3−/CD56+ cells with the combination of feeder cells, in the presence of IL-21 for a second period.

In some embodiments, IL-21 is added more than once during Day 0-6 of the second period.

In some embodiments, IL-21 and the combination of feeder cells are added more than once during Day 0-6 of the second period.

In some embodiments, IL-21 is added more than once during the first six days of every fourteen-day cycle during the second period.

In some embodiments, the method further comprises administration of one or more secondary Alzheimer's disease therapeutics.

In some embodiments, the one or more secondary Alzheimer's disease therapeutics comprises aducanumab, lecanemab, and/or donaneman.

In some embodiments, the NK cells and the one or more secondary Alzheimer's disease therapeutics are co-administered.

In some embodiments, the NK cells and the one or more secondary Alzheimer's disease therapeutics are administered for 1, 2, 3, 4, 5, 6, 7, 8, 9, 10, 11, 12, 14, 16, 20, 24, 28, 32, or 36 weeks.

In some embodiments, the NK cells and the one or more secondary Alzheimer's disease therapeutics are co-administered for 1, 2, 3, 4, 5, 6, 7, 8, 9, 10, 11, 12, 14, 16, 20, 24, 28, 32, or 36 weeks.

In some embodiments, the NK cells and the one or more secondary Alzheimer's disease therapeutics are alternately administered for 1, 2, 3, 4, 5, 6, 7, 8, 9, 10, 11, 12, 14, 16, 20, 24, 28, 32, or 36 weeks.

In some embodiments, administration of the NK cells in combination with one or more secondary Alzheimer's therapies and/or therapeutics, reduces the time required for the NK cells to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the time required for the one or more secondary Alzheimer's disease therapeutics to achieve a therapeutic effect by 1-fold, 2-fold, 3-fold, 4-fold, 5-fold, 6-fold, 7-fold, 8-fold, 9-fold, or 10-fold

In some embodiments, administration of the NK cells in combination with one or more secondary Alzheimer's therapies and/or therapeutics, reduces the time required for the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the time required for the NK cells to achieve a therapeutic effect by 1-fold, 2-fold, 3-fold, 4-fold, 5-fold, 6-fold, 7-fold, 8-fold, 9-fold, or 10-fold.

In some embodiments, administration of the NK cells in combination with one or more secondary Alzheimer's therapies and/or therapeutics, reduces the number of doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the number of doses of the NK cells required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the doses of the NK cells required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.

In some embodiments, administration of the NK cells in combination with one or more secondary Alzheimer's therapies and/or therapeutics, reduces the number of doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.

In some embodiments, administration of the NK cells in combination with one or more secondary Alzheimer's therapies and/or therapeutics, reduces the number of doses of the NK cells required to achieve a therapeutic effect by 1-fold, 2-fold, 3-fold, 4-fold, 5-fold, 6-fold, 7-fold, 8-fold, 9-fold, or 10-fold.

In some embodiments, administration of the NK cells in combination with one or more secondary Alzheimer's therapies and/or therapeutics, reduces the doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.

In some embodiments, administration of the NK cells in combination with one or more secondary Alzheimer's therapies and/or therapeutics, reduces the doses of the NK cells required to achieve a therapeutic effect by 1-fold, 2-fold, 3-fold, 4-fold, 5-fold, 6-fold, 7-fold, 8-fold, 9-fold, or 10-fold.

A kit comprising the NK cell population of any of the preceding embodiments and one or more secondary Alzheimer's disease therapeutics.

A formulation comprising the NK cell population of any of the preceding embodiments and one or more secondary Alzheimer's disease therapeutics.

A composition comprising the NK cell population of any of the preceding embodiments and one or more secondary Alzheimer's disease therapeutics.

The method of any one of the preceding embodiments, wherein identifying a subject as having Alzheimer's disease comprises detecting and/or quantifying one or more biomarkers.

The method of any one of the preceding embodiments, wherein the one or more biomarkers are quantified and/or detected in the subject's cerebrospinal fluid and/or blood.

The method of any one of the preceding embodiments, wherein the one or more biomarkers comprise YKL-40, CX3CL1, TNF-α, IL-6, IL-8, IL-12/IL-23p40, and/or sTREM2, or any combination thereof.

The method of any one of the preceding embodiments, wherein the one or more biomarkers comprise Aβ-42/40, Aβ-42, total Tau, pTau, pTau 181, GFAP, and/or NfL, or any combination thereof.

The method of any one of the preceding embodiments, wherein identifying a subject as having Alzheimer's disease comprises administering one or more cognitive assessments.

The method of any one of the preceding embodiments, wherein the one or more cognitive assessments comprises a Clinical Dementia Rating, Alzheimer's disease assessment scale-cognitive subscale, mini-mental status exam, or any combination thereof.

The method of any one of the preceding embodiments, wherein administration of the NK cells increases the Aβ-42 level in the subject's CSF and/or plasma by about 28-275%.

The method of any one of the preceding embodiments, wherein administration of the NK cells increases the Aβ-42/40 ratio in the subject's CSF and/or plasma by about 40-264%.

The method of any one of the preceding embodiments, wherein administration of the NK cells increases the IL-8 level in the subject's CSF and/or plasma by about 25-180%.

The method of any one of the preceding embodiments, wherein administration of the NK cells decreases the pTau level in the subject's CSF and/or plasma by about 21-84%.

The method of any one of the preceding embodiments, wherein administration of the NK cells decreases the GFAP level in the subject's CSF and/or plasma by about 36-95%.

The method of any one of the preceding embodiments, wherein administration of the NK cells decreases NfL level in the subject's CSF and/or plasma by about 4-71%.

The method of any one of the preceding embodiments, wherein administration of the NK cells increases the CX3CL1 level in the subject's CSF and/or plasma by about 18-231%.

The method of any one of the preceding embodiments, wherein administration of the NK cells decreases the IL-6 level in the subject's CSF and/or plasma by about 19-65%.

The method of any one of the preceding embodiments, wherein administration of the NK cells decreases the TNF-α level in the subject's CSF and/or plasma by about 42-96%.

The method of any one of the preceding embodiments, wherein administration of the NK cells decreases the IL-12/IL-23p40 ratio in the subject's CSF and/or plasma by about 7-53%.

The method of any one of the preceding embodiments, wherein administration of the NK cells decreases neuroinflammation in the subject as compared to the level of neuroinflammation in the subject prior to administration of the NK cells. Also provided is a method of reducing neuroinflammation (e.g., inflammation in the brain related to Alzheimer's Disease) by administering a therapeutically effective amount of the expanded NK cells of the present disclosure, to a subject in need thereof. In some embodiments, the subject has Alzheimer's Disease. In some embodiments, decreased or reduced neuroinflammation is measured based on a decrease in one or more biomarkers of neuroinflammation, as described herein.

The method of any one of the preceding embodiments, wherein administration of the NK cells decreases neuroinflammation in the subject by up to about 100% as compared to the level of neuroinflammation in the subject prior to administration of the NK cells.

In some embodiments, any of the preceding embodiments is achieved by administering an amount of NK cells, as disclosed herein, to the subject to achieve said score and/or result.

In some embodiments, the NK cells are administered intravenously.

In some embodiments, the NK cells are intravenously administered weekly for up to 20 weeks.

In some embodiments, any of the above steps can have further steps added between them. In some embodiments, any one or more of the above steps can be performed concurrently or out of the order provided herein.

### BRIEF DESCRIPTION OF THE DRAWINGS

FIG. 1 illustrates some embodiments of the improvement in spatial or sight skills of a subject with Alzheimer's disease after treatment with NK cells.

FIG. 2 is a flowchart depicting some embodiments of a method of treating Alzheimer's Disease in a subject.

FIG. 3 is a flowchart depicting some embodiments of a method of cell therapy.

FIG. 4 is a flowchart depicting some embodiments of a population of expanded NK cells.

FIG. 5 is a flowchart depicting some embodiments of a method of treating Alzheimer's Disease in a subject.

FIG. 6A is a bar graph depicting the change in Clinical Dementia Rating (CDR) of subjects treated with different doses of NK cells.

FIG. 6B is a line graph depicting the change in Clinical Dementia Rating (CDR) of subjects treated with different doses of NK cells.

FIG. 7A is a bar graph depicting the change in Alzheimer's disease assessment scale-cognitive subscale (ADAS-Cog) scores of subjects treated with different doses of NK cells.

FIG. 7B is a line graph depicting the change in Alzheimer's disease assessment scale-cognitive subscale (ADAS-Cog) scores of subjects treated with different doses of NK cells.

FIG. 8A is a bar graph depicting the change in Mini-Mental State Examination (MMSE) scores of subjects treated with different doses of NK cells.

FIG. 8B is a line graph depicting the change in Mini-Mental State Examination (MMSE) scores of subjects treated with different doses of NK cells.

FIG. 9A is a line graph depicting the average change in Aβ-42 levels in the cerebrospinal fluid of subjects treated with different doses of NK cells.

FIG. 9B is a line graph depicting the change in Aβ-42 levels in the cerebrospinal fluid of subjects treated with different doses of NK cells.

FIG. 10A is a line graph depicting the average change in Aβ-42/40 ratio in the cerebrospinal fluid of subject's treated with different doses of NK cells.

FIG. 10B is a line graph depicting the change in Aβ-42 levels in the cerebrospinal fluid of subject's treated with different doses of NK cells.

FIG. 11A is a line graph depicting the average change in total Tau levels in the cerebrospinal fluid of subjects treated with different doses of NK cells.

FIG. 11B is a line graph depicting the change in Aβ-42/40 ratio in the cerebrospinal fluid of subject's treated with different doses of NK cells.

FIG. 12A is a line graph depicting the average change in p-tau 181 levels in the cerebrospinal fluid of subjects treated with different doses of NK cells.

FIG. 12B is a line graph depicting the change in p-tau 181 levels in the cerebrospinal fluid of subjects treated with different doses of NK cells.

FIG. 13A is a line graph depicting the average change in GFAP levels in the cerebrospinal fluid of subjects treated with different doses of NK cells.

FIG. 13B is a line graph depicting the change in GFAP levels in the cerebrospinal fluid of subjects treated with different doses of NK cells.

FIG. 14A is a line graph depicting the average change in NfL levels in the cerebrospinal fluid of subjects treated with different doses of NK cells.

FIG. 14B is a line graph depicting the change in NfL levels in the cerebrospinal fluid of subjects treated with different doses of NK cells.

FIG. 15A is a line graph depicting the average change in YKL-40 levels in the cerebrospinal fluid of subjects treated with different doses of NK cells.

FIG. 15B is a line graph depicting the change in YKL-40 levels in the cerebrospinal fluid of subjects treated with different doses NK cells.

FIG. 16A is a line graph depicting the average change in baseline CX3CL1 (Fractalkine) levels in the cerebrospinal fluid of subjects treated with different doses of NK cells.

FIG. 16B is a line graph depicting the change in baseline CX3CL1 (Fractalkine) levels in the cerebrospinal fluid of subjects treated with different doses NK cells.

FIG. 17A is a line graph depicting the average change in baseline IL-6 levels in the cerebrospinal fluid of subjects treated with different doses of NK cells.

FIG. 17B is a line graph depicting the change in baseline IL-6 levels in the cerebrospinal fluid of subjects treated with different doses of NK cells.

FIG. 18A is a line graph depicting the average change in baseline TNF-α levels in the cerebrospinal fluid of subjects treated with different doses of NK cells.

FIG. 18B is a line graph depicting the change in baseline TNF-α levels in the cerebrospinal fluid of subjects treated with different doses of NK cells.

FIG. 19A is a line graph depicting the average change in baseline IL-8 levels in the cerebrospinal fluid of subjects treated with different doses of NK cells.

FIG. 19B is a line graph depicting the change in baseline IL-8 levels in the cerebrospinal fluid of subjects treated with different doses of NK cells.

FIG. 20A is a line graph depicting the average change in baseline IL-12/IL-23p40 levels in the cerebrospinal fluid of subjects treated with different doses of NK cells.

FIG. 20B is a line graph depicting the change in baseline IL-12/IL-23p40 levels in the cerebrospinal fluid of subjects treated with different doses of NK cells.

FIG. 21A is a line graph depicting the average change in baseline sTREM2 levels in the cerebrospinal fluid of subjects treated with different doses of NK cells.

FIG. 21B is a line graph depicting the change in baseline sTREM2 levels in the cerebrospinal fluid of subjects treated with different doses of NK cells.

FIG. 22A is a line graph showing the average expression level (percentage) of CX3CR1 in T cells in CSF of subjects treated with different doses of NK cells.

FIG. 22B is a line graph showing the expression level (percentage) of CX3CR1 in T cells in CSF of subjects treated with different doses of NK cells.

FIG. 23A is a line graph showing the average expression level (percentage) of CX3CR1 in NK cells in CSF of subjects treated with different doses of NK cells.

FIG. 23B is a line graph showing the expression level (percentage) of CX3CR1 in NK cells in CSF of subjects treated with different doses of NK cells.

FIG. 24A is a line graph showing the average expression level (percentage) of CX3CR1 in microglia in CSF of subjects treated with different doses of NK cells.

FIG. 24B is a line graph showing the expression level (percentage) of CX3CR1 in microglia in CSF of subjects treated with different doses of NK cells.

FIG. 25A is a bar graph depicting average NK cell activity in the plasma of subjects treated with different doses of NK cells.

FIG. 25B is a bar graph depicting NK cell activity in the plasma of subjects treated with different doses of NK cells.

FIG. 26 shows the Study Design for a dose escalation study of SNK01 administered to Alzheimer's Disease patients.

FIG. 27 shows a line graph depicting the Clinical Dementia Rating-Sum of Box (CDR-SB) scores of subjects treated with different doses of NK cells.

FIG. 28 shows a line graph depicting the mean change from baseline in the Clinical Dementia Rating-Sum of Box (CDR-SB) scores of subjects treated with different doses of NK cells.

FIG. 29 shows a line graph depicting the Alzheimer's Disease Assessment Scale-Cognitive subscale (ADAS-Cog) scores of subjects treated with NK cells.

FIG. 30 shows a line graph depicting the mean change from baseline for Alzheimer's Disease Assessment Scale-Cognitive subscale (ADAS-Cog) scores of subjects treated with different doses of NK cells.

FIG. 31 shows a line graph depicting the Mini-Mental State Examination (MMSE) scores of subjects treated with different doses of NK cells.

FIG. 32 shows a line graph depicting the mean change from baseline for the Mini-Mental State Examination (MMSE) scores of subjects treated with different doses of NK cells.

FIG. 33 shows a line graph depicting the change in Aβ-42 levels in the cerebrospinal fluid of subjects treated with different doses of NK cells.

FIG. 34 shows a line graph depicting the mean change from baseline for aggregate changes in Aβ-42 levels in the cerebrospinal fluid of subjects treated with different doses of NK cells.

FIG. 35 shows a line graph depicting the change in Aβ-42/40 ratio in the cerebrospinal fluid of subjects treated with different doses of NK cells.

FIG. 36 shows a line graph depicting the mean change from baseline in Aβ-42/40 ratio in the cerebrospinal fluid of subjects treated with different doses of NK cells.

FIG. 37 shows line graphs depicting the change in total tau levels in the cerebrospinal fluid of subjects treated with different doses of NK cells.

FIG. 38 shows a line graph depicting the change in p-tau 181 levels in the cerebrospinal fluid of subjects treated with different doses of NK cells.

FIG. 39 shows a line graph depicting the mean change from baseline in the aggregate change in p-tau 181 levels in the cerebrospinal fluid of subjects treated with different doses of NK cells.

FIG. 40 shows a line graph depicting the change in GFAP levels in the cerebrospinal fluid of subjects treated with different doses of NK cells.

FIG. 41 shows a line graph depicting the mean change from baseline in the aggregate change in GFAP levels in the cerebrospinal fluid of subjects treated with different doses of NK cells.

FIG. 42 shows a line graph depicting the change in NfL levels in the cerebrospinal fluid of subjects treated with different doses of NK cells.

FIG. 43 shows a line graph depicting the mean change from baseline in NfL levels in the cerebrospinal fluid of subjects treated with different doses of NK cells.

FIG. 44 shows a line graph depicting the change in YKL-40 levels in the cerebrospinal fluid of subjects treated with different doses of NK cells.

FIG. 45 shows a line graph depicting the mean change from baseline in YKL-40 levels in the cerebrospinal fluid of subjects treated with different doses of NK cells.

FIG. 46 shows a line graph depicting the change in baseline CX3CL1 (Fractalkine) levels in the cerebrospinal fluid of subjects treated with different doses of NK cells.

FIG. 47 shows line graphs depicting the change in baseline IL-6 levels in the cerebrospinal fluid of subjects treated with different doses of NK cells.

FIG. 48 shows line graphs depicting the change in baseline TNF-α levels in the cerebrospinal fluid of subjects treated with different doses of NK cells.

FIG. 49 shows line graphs of Aβ-42 changes in the plasma of subjects treated with NK cells.

FIG. 50 shows line graphs of Aβ-42/40 ratio changes in the plasma of subjects treated with NK cells.

FIG. 51 shows line graphs of changes in total Tau in the plasma of subjects treated with NK cells.

FIG. 52 shows line graphs of p-tau 181 changes in the plasma of subjects treated with NK cells.

FIG. 53 shows line graphs of GFAP changes in the plasma of subjects treated with NK cells.

FIG. 54 shows line graphs of NfL changes in the plasma of subjects treated with NK cells.

FIG. 55 shows line graphs of YKL-40 changes in the plasma of subjects treated with NK cells.

FIG. 56 shows line graphs of TNF-α changes in the plasma of subjects treated with NK cells.

FIG. 57 shows line graphs of IL-8 changes in the plasma of subjects treated with NK cells.

FIG. 58 shows line graphs of IL-6 changes in the plasma of subjects treated with NK cells.

FIG. 59 shows line graphs of IL-1β changes in the plasma of subjects treated with NK cells.

FIG. 60 shows line graphs of IL-10 changes in the plasma of subjects treated with NK cells.

FIG. 61 shows line graphs of IFN-γ changes in the plasma of subjects treated with NK cells.

FIG. 62 shows a line graph of the percentage of CD3+/CD56− T cells in the Leukocytes of subjects treated with NK cells.

FIG. 63 shows a line graph of the change from the baseline in frequency of CD3+/CD56− T cells in Leukocytes in subjects treated with NK cells.

FIG. 64 shows a line graph of the mean change from baseline in frequency of CD3+/CD56− T cells in Leukocytes in subjects treated with different doses of NK cells.

FIG. 65 shows a line graph of the percentage of CD3+/CD56− T cells in Lymphocytes of subjects treated with NK cells.

FIG. 66 shows a line of the change from the baseline in frequency of CD3+/CD56− T cells in Lymphocytes in subjects treated with NK cells.

FIG. 67 shows a line graph of the mean change from baseline in frequency of CD3+/CD56− T cells in Lymphocytes in subjects treated with different doses of NK cells.

FIG. 68 shows a line graph of the percentage of CX3CR1+ cells in CD3−CD56+ NK Cells from subjects treated with NK cells.

FIG. 69 shows a line graph of the change from the baseline in the percentage of CX3CR1+ cells in CD3−CD56+ NK Cells in subjects treated with NK cells.

FIG. 70 shows a line graph of the mean change from baseline in the percentage of CX3CR1+ cells in CD3−CD56+ NK Cells in subjects treated with different doses of NK cells.

FIG. 71 shows a line graph of the percentage of CX3CR1+ cells in CD3+CD56− T Cells from subjects treated with NK cells.

FIG. 72 shows a line graph of the change from the baseline in the percentage of CX3CR1+ cells in CD3+CD56− T Cells in subjects treated with NK cells.

FIG. 73 shows a line graph of the mean change from baseline in the percentage of CX3CR1+ cells in CD3+CD56− T Cells in subjects treated with different doses of NK cells.

FIG. 74 is a line graph depicting the change in total tau levels in the cerebrospinal fluid of subjects treated with different doses of NK cells.

FIG. 74 shows a line graph of the mean change from baseline in total tau in CSF of subjects receiving different doses of NK cells.

FIG. 75 show a bar graph depicting the change in Clinical Dementia Rating (CDR) of subjects treated with different doses of NK cells.

FIG. 76 is a bar graph depicting the change in Alzheimer's disease assessment scale-cognitive subscale (ADAS-Cog) scores of subjects treated with different doses of NK cells.

FIG. 77 is a line graph depicting the change in Alzheimer's disease assessment scale-cognitive subscale (ADAS-Cog) scores of subjects treated with different doses of NK cells.

FIG. 78 is a bar graph depicting the change in Mini-Mental State Examination (MMSE) scores of subjects treated with different doses of NK cells.

FIG. 79 is a line graph depicting the change in Aβ-42 levels in the cerebrospinal fluid of subjects treated with different doses of NK cells.

### DETAILED DESCRIPTION

Natural killer cells (NK cells) are one type of innate immune cells, which are known to recognize and kill virus-infected and tumor cells by releasing cytotoxic granules such as perforin and granzyme or by death receptor-mediated cytotoxicity. NK cells have also been reported to be able to kill activated T cells (Rabinovich B, et al., J Immunol, 2003: 170: 3572-3576).

In order to obtain the therapeutic effect of NK cells in Alzheimer's disease, a large amount of NK cells having high purity is useful, but it is not easy to obtain a large amount of blood from the Alzheimer's patient, and the proportion of NK cells in the blood is small, only about 5 to 20%. Thus, it has been difficult to use NK cells as an immunotherapeutic agent.

It is desirable to effectively activate and proliferate only the NK cells; however, in a conventional method of proliferating NK cells, various expensive cytokines need to be used at a high concentration, thus the corresponding therapy is only available to some financially stable patients. Further, according to conventional methods of proliferating NK cells, other types (e.g., T cells, B cells, etc.) of immune cells may be present together with the NK cells, and allogeneic administration of the NK cells containing T cells may cause a graft versus host disease (GVHD) and allogeneic administration of NK cells containing B cells to blood-type incompatible subjects may cause a passenger B-lymphocyte syndrome, and thus, the therapeutic effect of NK cells in Alzheimer's disease is not maximized.

Further, in addition to activating and proliferating NK cells, it is desirable to highly maintain the functions of NK cells until the activated and proliferated NK cells are clinically used. As a result, the development of a composition capable of promoting the proliferation of the NK cells, increasing production of cytokines such as TNFα, IFNγ and GM-CSF derived from the NK cells, and increasing activity of the NK cells is sought.

Provided herein are methods and compositions for treating Alzheimer's disease comprising natural killer cells, which can be highly pure and/or present in large amounts and/or especially active.

### Terminology

All terms are to be given their ordinary and customary meaning as understood by one of ordinary skill in the art, in view of the present disclosure.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The ranges disclosed herein also encompass any and all overlap, sub-ranges, and combinations thereof. Language such as “up to,” “at least,” “greater than,” “less than,” “between,” and the like includes the number recited. The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about 5.0 cm” includes “5.0 cm.”

Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The term “generally” as used herein represents a value, amount, or characteristic that predominantly includes or tends toward a particular value, amount, or characteristic. As an example, in certain embodiments, the term “generally uniform” refers to a value, amount, or characteristic that departs from exactly uniform by less than 20%, less than 15%, less than 10%, less than 5%, less than 1%, less than 0.1%, and less than 0.01%.

The term “treat” and “treatment” includes therapeutic treatments, prophylactic treatments, and applications in which one reduces the risk that a subject will develop a disorder or other risk factor. As used herein, the term “treatment” includes an intervention (e.g., a clinical intervention) made in response to a disease, disorder or physiological condition manifested by a patient, particularly a patient suffering from a neuro degenerative disease, for example Alzheimer's disease. Treatment does not require the complete curing of a disorder and encompasses embodiments in which one reduces symptoms or underlying risk factors, slowing or stopping the progression or worsening of a disease, disorder, or condition and the remission of the disease. Symptoms of Alzheimer's disease include but are not limited to a motor deficit and cognitive problems such as memory loss, disorientation, decline in ability to perform routine tasks, difficulty learning, loss of language skills, depression, agitation and dementia. The “treatment” of Alzheimer's disease comprises reduction, slowing or stopping one or more symptoms associated with Alzheimer's disease.

In some embodiments, the cell therapeutic composition may include a therapeutically effective amount of cell therapeutic agent for treatment of diseases. The term “therapeutically effective amount” means an amount of an active ingredient or a cell therapeutic composition which induces biological or medical responses in tissue systems, animals, or humans which are considered by researchers, veterinarians, physicians, or other clinicians, and includes an amount of inducing alleviation of symptoms of diseases or disorders to be treated. It will be apparent to those skilled in the art that the cell therapeutic agent included in the cell therapeutic composition may be changed according to a desired effect. Therefore, the optimal content of the cell therapeutic agent may be easily determined by those skilled in the art, and may be adjusted according to various factors including a type of disease, severity of the disease, contents of other ingredients contained in the composition, a type of formulation, and an age, a weight, a general health condition, a gender, and a diet of a patient, an administration time, an administration route, a secretion ratio of the composition, a treatment period, and simultaneously used drugs. It is important to include an amount capable of obtaining a maximum effect by a minimum amount without side effects by considering all of the factors. For example, in some embodiments, the cell therapeutic composition may include a cell therapeutic agent of 1×10⁶ to 5×10⁸ cells per kg of body weight.

As used herein, the term “Alzheimer's disease” refers to the neurodegenerative disease that causes loss of memory, dementia, and eventual death. A common hypothesized cause is the aggregation and accumulation of amyloid beta (Aβ) peptides in the brain, resulting in neuronal degeneration and inflammation. Currently available therapeutics, such as cholinesterase inhibitors and NDMA receptor antagonists, have minimal effect on the progression of the disease and/or only treat secondary symptoms of the disease. “Alzheimer's disease” and “Alzheimer's” is used interchangeably herein.

As used herein, the term “dementia” refers to a general term for loss of memory, language, problem-solving and other thinking abilities that are severe enough to interfere with daily life. Alzheimer's is the most common cause of dementia. Disorders grouped under the general term “dementia” are caused by abnormal brain changes. These changes trigger a decline in thinking skills, also known as cognitive abilities, severe enough to impair daily life and independent function. They also affect behavior, feelings and relationships. Types of dementia include Alzheimer's disease, vascular dementia, Dementia With Lewy Bodies (DLB), Parkinson's Disease Dementia. Mixed Dementia, Frontotemporal Dementia (FTD), Huntington's Disease, Creutzfeldt-Jakob Disease, normal pressure hydrocephalus, and Wemicke-Korsakoff Syndrome. Alzheimer's disease accounts for 60-80% of cases. Vascular dementia, which occurs because of microscopic bleeding and blood vessel blockage in the brain, is the second most common cause of dementia. Those who experience the brain changes of multiple types of dementia simultaneously have mixed dementia. There are many other conditions that can cause symptoms of dementia, including some that are reversible, such as thyroid problems and vitamin deficiencies. Signs of dementia can vary greatly. Examples include problems with: short-term memory; keeping track of a purse or wallet; paying bills; planning and preparing meals; remembering appointments; and/or traveling out of the neighborhood.

Dementia is caused by damage to brain cells. This damage interferes with the ability of brain cells to communicate with each other. When brain cells cannot communicate normally, thinking, behavior and feelings can be affected. Different types of dementia are associated with particular types of brain cell damage in particular regions of the brain. For example, in Alzheimer's disease, high levels of certain proteins inside and outside brain cells make it hard for brain cells to stay healthy and to communicate with each other. The brain region called the hippocampus is the center of learning and memory in the brain, and the brain cells in this region are often the first to be damaged. That's why memory loss is often one of the earliest symptoms of Alzheimer's. While most changes in the brain that cause dementia are permanent and worsen over time, thinking and memory problems caused by the following conditions may improve when the condition is treated or addressed: depression; medication side effects; excess use of alcohol; thyroid problems; vitamin deficiencies; diagnosis of dementia.

There is no one test to determine if someone has dementia. Doctors diagnose Alzheimer's and other types of dementia based on a careful medical history, a physical examination, laboratory tests, and the characteristic changes in thinking, day-to-day function and behavior associated with each type. Doctors can determine that a person has dementia with a high level of certainty. But it's harder to determine the exact type of dementia because the symptoms and brain changes of different dementias can overlap. In some cases, a doctor may diagnose “dementia” and not specify a type. If this occurs, it may be necessary to see a specialist such as a neurologist, psychiatrist, psychologist or geriatrician. The brain has many distinct regions, each of which is responsible for different functions (for example, memory, judgment and movement). When cells in a particular region are damaged, that region cannot carry out its functions normally.

As used herein, the term Mini-Mental State Examination Scale (MMSE) refers to a brief 30-point questionnaire used to assess cognitive impairment with lower scores indicating greater impairment. The Mini-Mental State Examination (MMSE) is the best-known and the most often used short screening tool for providing an overall measure of cognitive impairment in clinical, research and community settings. The MMSE assesses 11 categories of cognition including orientation to time, memory, attention, concentration, naming, repetition, comprehension, and the ability to create a sentence and to copy 2 intersecting polygons. The total scores on the scale ranges from 0 to 30 with lower scores indicating greater impairment.

As used herein, the term Clinical Dementia Rating (CDR) refers to a global assessment instrument that yields global score and a sum of boxes score. The Clinical Dementia Rating Scale is derived from a semi-structured interview with the participant and an appropriate informant, and it rates impairment in 6 categories (memory, orientation, judgment, and problem solving, community affairs, home and hobbies, and personal care) on a 5-point scale for which 0=no impairment, 0.5=questionable impairment, and 1, 2, and 3=mild, moderate, and severe impairment, respectively. From the 6 individual category ratings, or box scores, the Clinical Dementia Rating Scale—Global Score is established by clinical scoring rules, for which the Clinical Dementia Rating of 0=no dementia and Clinical Dementia Rating of 0.5, 1, 2, or 3=questionable, mild, moderate, or severe dementia, respectively (Morris, 1993). The CDR-SB score is a detailed quantitative general index that provides more information than the Clinical Dementia Rating Scale—Global Score in participants with early (prodromal to mild) dementia (Cedarbaum et al, 2013; Coley et al, 2011). In particular, the CDR-SB has been proposed for use in longitudinal assessment of dementia and is widely used in AD studies as a global measure of disease progression (Williams et al, 2013).

As used herein, the term “Alzheimer's Disease Assessment Scale” (ADAS) refers to a rating scale for assessing the severity of cognitive (ADAS-cog) and non-cognitive dysfunction resulting from mild to severe AD. The ADAS is scored by summing the number of errors made on each task so that higher scores indicate worse performance. The ADAS comprises two subscales. The non-cognitive subscale (ADAS-Noncog) includes 10 tasks, which consider mood and behavioral changes. The cognitive subscale (ADAS-Cog) includes both subject-completed tests and observer-based assessments. Specific tasks include word recall, naming objects and fingers, commands, constructional praxis, ideational praxis, orientation, word recognition, and language. Together these tasks assess the cognitive domains of memory, language, and praxis.

Any suitable option for assessing the severity of AD can be used in any embodiment of the present disclosure. In some embodiments, the cognitive assessment is suitable for severe AD including, without limitation, Clinical Dementia Rating Scale Sum of Boxes (CDR-SB), Mini-mental status exam (MMSE), Neuropsychiatric Inventory (NPI), Alzheimer's Disease Cooperative Study Group Clinical Global Impression of Change Caregiver Input (ADCS-CGIC), Alzheimer's Disease Assessment Scale—Cognitive Subscale (ADAS-Cog), Severe Impairment Battery (SIB), and Alzheimer's Disease Cooperative Study Group Activities of Daily Living Inventory for Severe Alzheimer's Disease (ADCS-ADL-Severe).

As used herein, the term “amyloid” refers to an aggregated protein structure consisting of unbranched microscopic fibrils often found in dense tissue deposits and associated with a variety of human diseases, including a number of significant neurodegenerative disorders. “Amyloid Beta” (Aβ) is produced from a transmembrane Aβ precursor protein (APP), APP is sequentially cleaved by β- and γ-secretase. Cleavage of APP by γ-secretase generates a number of AD isoforms, among which Aβ42 has the highest propensity for aggregation and appears to be the predominant species in neuritic plaques. Decreased CSF Aβ42 has been consistently found in the CSF of AD patients. Studies have demonstrated inverse correlations between CSF Aβ42 and neuritic plaque burden suggesting that low levels of Aβ42 in CSF are caused by its deposition in the brain parenchyma. (Janelidze et al. 2016). CSF Aβ42/Aβ40 ratio is a diagnostic biomarker of AD during both predementia and dementia stages in comparison to CSF Aβ42 alone. The ratios reflect AD-type pathology better, whereas decline in CSF Aβ42 is also associated with non-AD subcortical pathologies. Studies suggested that the ratios rather than CSF Aβ42 can be used in the clinical work-up of AD. (Janelidze, Shorena et al. “CSF Aβ42/Aβ40 and Aβ42/Aβ38 ratios: better diagnostic markers of Alzheimer disease.” Annals of clinical and translational neurology vol. 3, 3 154-65. 1 Jan. 2016, doi:10.1002/acn3.274).

As used herein, the term “Tau” refers to an intracellular protein that contributes to the assembly and stabilization of microtubules in neuronal axons. In AD, tau becomes hyperphosphorylated and loses its ability to assemble and stabilize microtubules. CSF Total tau (t-tau) concentration shows a marked increase in AD, reflecting the neuronal damage associated with the disease. Increase in t-tau concentration reflects the degree of neuronal degeneration. (Randall et al. 2013).

The tangles characteristic of AD are made up of filaments formed from an abnormally phosphorylated form of tau called “phospho-tau” (p-tau). P-tau is believed to reflect neurofibrillary pathology. P-tau is more specific to AD than t-tau and it allows for better differentiation between AD and other neurodegenerative diseases. (Randall et al. 2013) P-tau 217 and p-tau 181 in cerebrospinal fluid (CSF) and plasma have been used as biomarkers of tau-pathology. P-tau 217 and p-tau 181 levels correlate to cognitive impairment in AD.

As used herein, the term “glial fibrillary acidic protein” (GFAP) refers to an intermediate filament structural protein involved in cytoskeleton assembly and integrity, expressed in high abundance in activated glial cells. Neuronal stress, caused by either disease or injury, evokes astrocyte activation as a response, including hypertrophy, proliferation, and increased GFAP expression. Glial fibrillary acidic protein (GFAP) is a marker of reactive astrogliosis that increases in the cerebrospinal fluid (CSF) and blood of individuals with Alzheimer disease (AD) (Ganne, Akshatha et al. “Glial Fibrillary Acidic Protein: A Biomarker and Drug Target for Alzheimer's Disease.” Pharmaceutics vol. 14, 7 1354. 26 Jun. 2022, doi:10.3390/pharmaceutics14071354).

As used herein, the term “YKL-40” (also known as Chitinase 3-like 1) refers to a glycoprotein produced by inflammatory, cancer and stem cells. YKL-40 is elevated in the brain and cerebrospinal fluid (CSF) in several neurological and neurodegenerative diseases associated with inflammatory processes. YKL-40 quantification is expected to have an application in the evaluation of therapeutic intervention in dementias with a neuroinflammatory component. (Llorens, F., Thüfne, K., Tahir, W. et al. YKL-40 in the brain and cerebrospinal fluid of neurodegenerative dementias. Mol Neurodegeneration 12, 83 (2017).

As used herein, the term “CX3CL1” or “Fractalkine” refers to a chemokine expressed mainly in neurons in the CNS. Soluble CX3CL1 has a chemoattractive effect for monocytes, natural killer cells, and lymphocyte cells. Receptor CX3CR1 is expressed in microglia, astrocytes, T cells and NK cells. The interaction between CX3CL1 and CX3CR1 has both beneficial and detrimental consequences throughout the activation of various pathways within microglia. Therefore, correct functionality of the CX3CL1/CX3CR1 axis is crucial for the maintenance of brain homeostasis, and especially for dealing with microglia-mediated inflammation in the CNS. CX3CL1 acts as a regulator of microglia activation in response to brain injury or inflammation. Low level of CX3CL1 concentrations were found in the CSF of AD patients. (Perea et al. 2018, González-Prieto et al. 2021, Pawelec et al. 2020).

As used herein, the term “Soluble triggering receptor expressed on myeloid cells 2” (sTREM2) is a protein expressed on macrophages, immature monocyte-derived dendritic cells, osteoclasts, and microglia. sTREM2 in cerebrospinal fluid (CSF) has been described as a biomarker for microglial activation, which has been seen to be increased in a variety of neurological disorders. For example, an early increase of CSF sTREM2 in Alzheimer's disease is associated with tau related-neurodegeneration but not with amyloid-β pathology, relating to microglial function and to cognitive impairment in AD.

As used herein, the term “neurofilament light chain” (NfL) refers to a neuronal cytoplasmic protein highly expressed in large calibre myelinated axons. NfL is found particularly in neuronal axons, keeping the myelinated axons structurally stable and playing an essential role in the growth and impulse conduction along the axons. They also act as skeletal supports, helping to maintain the shape of neurons. The levels of Cerebrospinal fluid (CSF) neurofilament light (NfL) are significantly elevated in AD and is a biomarker of neurodegeneration. (Dhiman et al. 2020).

### Embodiments

In some embodiments, the therapeutic composition comprises the expanded NK cells (of any of the embodiments provided herein) in combination with one or more additional (or “secondary”) therapeutics. In some embodiments, the one or more secondary therapeutics are any one or more therapeutics for use in the treatment of Alzheimer's disease. In some embodiments, the one or more secondary therapeutics comprise an immunotherapy, a passive immunotherapy, an active immunotherapy, a small molecule, a supplement, a dietary supplement, a DNA based therapy, an RNA based therapy, a small molecule, or other secondary Alzheimer's disease therapy, or is a combination of one or more of the preceding therapeutics.

In some embodiments, the one or more secondary Alzheimer's disease therapies is administered orally, intravenously, via injection, via any suitable means, or via any combination therein. In some embodiments, the NK cells and the one or more secondary Alzheimer's disease therapies are administered in the same way. In some embodiments, the NK cells and the one or more secondary Alzheimer's disease therapies are administered in different ways.

In some embodiments, the one or more secondary Alzheimer's therapeutics comprise an FDA approved Alzheimer's therapeutic. In some embodiments, the one or more secondary therapeutics comprise aducanumab, lecanemab, and/or donaneman, or any combination thereof. In some embodiments, the subject undergoes magnetic resonance imaging prior to administration of the aducanumab. In some embodiments, the aducanumab is administered by IV infusion. In some embodiments, the aducanumab is administered by IV infusion over 1, 2, 3, 4, 5, 6, 7, 8, 9, 10, 11, 12, 13, 14, 15, 16, 17, 18, 19, 20, 21, 22, 23, or 24 hours, or by a range that is defined by any two of the preceding values. In some embodiments the aducanumab is administered at 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0, mg/kg, or by a range that is defined by any two of the preceding values. In some embodiments, the aducanumab is administered every 1, 2, 3, 4, 5, 6, 7, 10, 14, 15, 20, 21, 28, 29, 30, or 31, days, or by a range that is defined by any two of the preceding values. In some embodiments, the number of doses of aducanumab administered is 1, 2, 3, 4, 5, 6, 7, 8, 9, 10, 11, 12, 13, 14, 15, 16, 17, 18, 19, 20, 21, 22, 23, 24, 25, 26, 27, or 28, or is a range that is defined by any two of the preceding values. In some embodiments, the dosage of aducanumab administered is titrated over time. For example, in some embodiments, the dosage of aducanumab administered is increased by about 1%, 2%, 3%, 4%, %5, 6%, 7%, 8%, 9%, 10%, 15%, 20% 25%, 30%, 40%, 50%, 60%, 70%, 75%, 80%, 90%, 91%, 92%, 93%, 94%, 95%, 96%, 97%, 98%, 99%, or 100%, or by a range that is defined by any two of the preceding values. In some embodiments, the dosage of aducanumab administered is increased by about 1-fold, 2-fold, 3-fold, 4-fold, 5-fold, 6-fold, 7-fold, 8-fold, 9-fold, or 10-fold, or by a range that is defined by any two of the preceding values.

In some embodiments, the one or more secondary therapeutics comprise AAB-003, PF-05236812, AADvac1, Axon peptide 108 conjugated to KLH, ABT 418, ABT-089, ABT-288, ABT-384, ABT-957, ABvac 40, ACI-24, Pal1-15 acetate salt, ACI-3024, Tau Morphomer™, ACI-35, VAC20121, ACU193, ACU-193, AF 102B, cevimeline HCL, Evoxac™, AL002, AL003, AL101, GSK-4527226, ALZ-101, ALZ-801, valiltramiprosate, NRM-8499, homotaurine prodrug, 3-APS, ALZT-OP1 Cromolyn, sodium, Intal, Ibuprofen, AN-1792, AIP 001, APNmAb005, RAA7, AR1001, ASN51, ASN121151, AVP-786, AVP-923, Nuedexta, Zenvia, AXS-05, Dextromethorphan/bupropion, AZD1446, TC-6683, AZD3480, ispronicline, TC-1734, AZP2006, AZP-2006, Acetyl-l-camitine HCIALCAR, Acitretin, Soriatane, Neotigason, RO 101670, Aduhelm, Aducanumab, BIIB037, Affitope AD02, Albrioza, AMX0035, Allopregnanolone, brexanolone, 3α-hydroxy-5α-pregnan-20-one, 3α,5α-tetrahydroprogesterone, Alpha-Tocopherol, Vitamin E, Alzhemed™, Vivimind™, Tramiprosate, NC-531, homotaurine, 3-APS, milomotide, CAD106, Aripiprazole, Abilify, BMS-337039, Atabecestat, JNJ-54861911, BACE inhibitor, Atomoxetine, ATX, Strattera, Atorvastatin, Lipitor™, Zarator®, Sortis®, Tahor®, Atuzaginstat, COR388, Avagacestat, BMS-708163, Azeliragon, PF-04494700, TTP488, BI 1181181, VTP 37948, BACE inhibitor, BI 409306, SUB 166499, BI 425809, BIIB076, NI-105, 6C5 huIgG1/1, BIIB080, IONIS-MAPTRx, ISIS 814907, BIIB118, PF-05251749, BPN14770, Bapineuzumab, AAB-001, Baricitinib, Olumiant®, NCB28050, LY3009104, Bepranemab, UCB0107, UCB 0107, Antibody D, Besipirdine HCl HP 749, Bexarotene, Targretin®, Blarcamesine, Anavex 2-73, Bosutinib, BOSULIF®, PF-5208763, SKI-606, Brexpiprazole, Rexulti, OPC34712, Bryostatin 1, CERE-110, Nerve Growth Factor gene therapy, CHF 5074, COGNIShunt™, CX516, Ampalex, Candesartan, Atacand, Candesartan cilexetil, Cannabidiol, CBD, Epidiolex, Carvedilol, Coreg, Artist, Aucardic, Dilatrend, Kredex, Celecoxib, Celebrex, Cerebrolysin, Circadin, Citalopram, escitalopram, Celexa, Lexapro, Cipralex, Clioquinol, iodochlorhydroxyquin, PBT-1, Continuous Positive Airway Pressure, CPAP, Contraloid, PRI-002, contraloid acetate, RD2, Crenezumab MABT5102A, RG7412, Curcumin, diferuloylmethane, Longvida™, DAYVIGO, Lemborexant, E2006, DNL747, SAR 443060, Dapagliflozin, Farxiga, Forxiga, BMS 512148, Dapsone, Avlosulfon, Diaminodiphenylsulfone, DDS, Dasatinib+Quercetin, Deep Brain Stimulation-fomix, Deferiprone, Ferriprox, Dexpramipexole, R-pramipexole, RPPX, KNS-760704, BIIB 050, Dimebon, Dimebolin, Latrepirdine, Pf-01913539, Docosahexaenoic acid (DHA), Omega 3 fatty acid, Donanemab, N3pG-Aβ Monoclonal Antibody, LY3002813, Donepezil Aricept™, Donepezil hydrochloride, Eranz®, E 2020, Dronabinol, THC, Marinol, Syndros, delta-9-tetrahydrocannabinol, delta-9-THC, E2814, EHT 0202, Etazolate, ELND005, AZD-103, Scyllo-inositol, cyclohexane-1,2,3,4,5,6-hexol, EVP-0962, EVP 0015962, Edicotinib, JNJ-40346527, JNJ-527, PRV-6527, Edonerpic, T-817 MA, T 817, Elayta, CT1812, Elenbecestat, E2609, BACE inhibitor, Empagliflozin, Jardiance, BI-10773, Encenicline, EVP-6124, MT-4666, α7-nAChR agonist, Epigallocatechin Gallate (EGCG), Sunphenon EGCg, Epothilone D, BMS-241027, Eptastigmine, MF 201, Estrogen, Premarin™, Etanercept, Enbrel™, Exenatide, Exendin-4, Byetta, Bydureon, Flurizan™, tarenflurbil, r-flurbiprofen, MPC-7869, Fosgonimeton, ATH-1017, NDX-1017, G-CSF, Filgrastim, GC 021109, GENUS, Gamma entrainment using sensory stimuli, GammaSense Stimulation, GLN-1062, Memogain, GRF6019, GSK239512, GSK2647544, GSK-2647544, GSK933776, GV-971, sodium oligomannate, sodium oligo-mannurarate, GV1001, RIAVAX™, Tertomotide, Galantamine, Razadyne™, Reminyl™, Nivalin®, Gammagard®, Intravenous Immunoglobulin, IVIg, Gamunex, Human Albumin Combined With Flebogamma, Gantenerumab, RO4909832, RG1450, Gemfibrozil, Lopid, Jezil, Gen-Fibro, Gosuranemab, BIIB092, BMS-986168, IPN007, Guanfacine, Intuniv, SPD503, Afken, Estulic, Tenex, HF0220, HTL0018318, Huperzine A, ZT-1, DEBIO 9902, Qian ceng ta, Cerebra capsule, Pharmassure Memorall capsule, Advil™, Nuprin™, Motrin™, Idalopirdine, Lu AE58054, SGS 518, Idebenone, Catena, Sovrima, Intepirdine RVT-101, SB 742457, GSK 742457, Inzomelid, JNJ-63733657, KarXT, xanomeline-trospium, Ketasyn, Axona, Caprylic Acid, AC-1202, LM11A-31-BHS, LM11A-31, LMTM, TRx0237, LMT-X, Methylene Blue, Tau aggregation inhibitor (TAI), LU25-109, LX1001, AAVrh.10-APOE2, AAVrh.10hAPOE2, LY2599666, LY2886721, BACE inhibitor, LY3202626, BACE Inhibitor, LY3372689, LY3372993, N3pG-Abeta mAb, Ladostigil, Ladostigil hemitartrate, TV3326, Lanabecestat, AZD3293, LY3314814, BACE inhibitor, Lecanemab, BAN2401, mAb158, Lenalidomide, Revlimid, Leuprolide, Leuprolide acetate, Lupron Depot, Eligard, Levetiracetam, Keppra, Linopirdine, DuP996, Liraglutide, Victoza™, Saxenda™, Lomecel-B, mesenchymal stem cells, Lomoxican, Losartan, Cozaar®, MK0954, Lu AF20513, Lu AF87908, Lumateperone M-007, MEDI1814, MEM 1003, BAY Z 4406, MK-7622, MKC-231, MSDC-0160, Mitoglitazone, MW150, MW01-18-150SRM, MW151, MW01-2-151SRM, Minozac, MW01-2-151WH, compound 17, Masitinib, Masivet, Kinavet, AB1010, Masitinib mesylate, Masupirdine, SUVN-502, Melatonin, Memantine, Ebixa™, Namenda™, Axura®, Akatinol®, Memary®, Metformin, Glucophage, Glucophage XR, Methylphenidate, Ritalin, Concerta, Metrifonate, trichlorfon, Milameline, CI 979, Minocycline, Solodyn, Arestin, Minocin, Dynacin, Montelukast, Singulair, MK0476, NGX267, AF267B, NIC5-15, Pinitol, D-Pinitol, NS2330, Tesofensine, Nabilone, Cesamet, Naproxen, Aleve™, Anaprox™, Naprosyn™, Nasal Insulin, Detemir, Levemir, Humulin, Novolin, glulisine, Nefiracetam, Neflamapimod, VX-745, Nelonicline, ABT-126, NeoTrofin, AIT-082, leteprinim, Neramexane, MRZ 2/579, Nicotinamide Riboside, Niagen, NR, Nicotinamide, Vitamin B3, Nilotinib, Tasigna, AMN107, Nilvadipine Nilvad, Nivadil, ARC029, Nuplazid, Pimavanserin, ACP-103, Pimavanserin tartrate, ORM-12741, Octagam®10%, NewGam, PBT2, PBT-2, PF-05212377, PF-5212377, WYE-103760, SAM-760, PF-06648671, PF-06751979, BACE inhibitor, PF-06852231, PNT001, PRX-03140, Potassium salt, PU-AD, PU-HZ151, icapamespib, PXT864, PXT00864, Pepinemab, VX15, VX15/2503, Phenserine, Physostigmine Salicylate, Synapton, Piromelatine, Neu-P11, Ponezumab, PF-04360365, Posiphen, ANVS-401, Posiphen tartrate, Prazosin, Prazosin hydrochloride, Minipress, Hypovase, Vasoflex, Prednisone, Propentofylline, HWA 285, PPF, Protollin, RG3487, RO5313534, MEM 3454, RG7129, RO5508887, BACE Inhibitor, RG7345, RO6926496, RO7126209, RG6102, Brain shuttle gantenerumab, Rasagiline, Rasagiline mesylate, Azilect, TVP-1012, Rember™, Methylene Blue, methylthioninium (MT), TRx-0014, Repetitive Transcranial Magnetic Stimulation, rTMS, Resveratrol, trans-3,4′,5-trihydroxystilbene, Rilapladib, SB-659032, Riluzole, Rilutek®, RP 54274, Rivastigmine Exelon™, Rivastigmine tartrate, Rivastach® Patch, Prometax®, SDZ ENA 713, Rofecoxib, Vioxx™, Rosiglitazone, Rosiglitazone maleate, Avandia, Rotigotine, Neupro, S 38093, S-Equol, Aus-131, S47445, CX1632, SAGE-718, SAR110894D, SAR228810, SB 202026, Memric, SDI-118, SGS-742, CGP-36742, DVD-742, ST101, ZSET1446, SUVN-G3031, Sabeluzole, R58735, Saracatinib, AZD0530, Sargramostim, GM-CSF Leukine, Leukine®, Semagacestat, LY450139 Dihydrate, hydroxylvaleryl monobenzocaprolactam, Semaglutide Ozempic, Rybelsus, Sembragiline, RO4602522, RG1577, Semorinemab, RO7105705, MTAU9937A, RG6100, Simufilam, PTI-125, sumifilam, Simvastatin, Zocor®, Lipex®, Lipovas®, Denan®, Solanezumab LY2062430, Souvenaid, Fortasyn Connect, Suritozole, MD 26479, Suvorexant, Belsomra, MK-4305, T3D-959, DB959, TB006, TPI 287, Tacrine, Cognex™, Telmisartan, Micardis, Thalidomide, Thalomid®, Tideglusib, NP031112, Nypta®, Zentylor™, Glycogen synthase kinase 3 inhibitor, NP12, Tilavonemab, ABBV-8E12, C2N 8E12, HJ8.5, Trazodone, Trazodone hydrochloride, Oleptro, Desyrel, Tricaprilin, AC-1204, Caprylic triglyceride, Troriluzole, BHV-4157, trigriluzole, FC-4157, UB-311, Umibecestat, CNP520, BACE Inhibitor, Vafidemstat, ORY-2001, Valacyclovir, Valtrex, Valaciclovir, 256U87, Valproate, Depakote, Depakene, Valproic acid, Divalproex sodium, Vanutide cridificar, ACC-001, PF-05236806, Varenicline, Champix™, Chantix™, Varenicline, tartrate, CP-526555, Alpha4 beta2 nicotinic receptor agonist, Varoglutamstat, PQ912, Vascepa, Icosapent ethyl (IPE), Ethyl eicosapentaenoic acid (E-EPA), AMR101, Miraxion, Verubecestat, MK-8931, MK-8931-009, BACE inhibitor, XPro1595 Pegipanermin, INB03, NeuLiv, XENP1595, DN-TNF, XENP345, XPro™, LIVNate, Xaliproden, SR 57746A, Xanamem, UE 2343, Young Plasma, Zagotenemab, LY3303560, kMCT-ONS, medium chain triglyceride-based ketogenic oral nutritional supplement, kMCT, and/or a combination therein. See alzforum(dot)org/therapeutics/search?fda_statuses=&target_types=&therapy_types=&conditions%5B%5D=145&keywords-entry=&keywords=#results.

In some embodiments, the expanded NK cells are administered in numerous doses, including up to 20 doses or more, such as 1, 2, 3, 4, 5, 6, 7, 8, 9, 10, 11, 12, 13, 14, 15, 16, 17, 18, 19 or 20 or more doses in a given period. In some embodiments, the method comprises administering the doses about 1, 2, 3, 4, 5, 6, 7, 8, 9, 10, 11, 12 weeks apart, including ranges between any two of the listed values. In some embodiments, the doses can be administered about 1, 2, 3, 4, 5, 6, 7, 8, 9, 10, 11, 12 months apart, including ranges between any two of the listed values. In some embodiments, the period for doses is monthly, quarterly, every 6 months, yearly, every 2, 3, 4, 5, or more years.

Some embodiments have been described wherein treatment of Alzheimer's disease with expanded NK cells leads to a slowing of the progression of the disease, improvement of symptoms or a reversal in the progression of Alzheimer's disease as measured by Mini-Mental State Exam (MMSE), Mini-Cog test, Global Deterioration Scale (GDS), Cognitive subscale (ADAS-Cog), Clinical Dementia rating scale: sum of boxes (CDR-SB), AD composite score (ADCOMS) and/or by measuring the change in one or more CSF biomarkers from the beginning to the end of a study or treatment method. For example, in certain embodiments, the treatment with expanded NK cells leads to an improvement in the symptoms or reversal in the progression of disease of at least about 10, 20, 30, 40, 50, 60, 70, 80, 90, 100%, including ranges between any of the listed values. In some embodiments, the CSF biomarker is a core CSF biomarker, a CSF inflammatory biomarker, a CSF immune cell chemokine ligand, a CSF innate immune receptor, and/or any combination thereof. In some embodiments, the CSF core biomarker comprises amyloid beta 42, amyloid beta 40, amyloid beta 42/40 ratio, total Tau (t-tau), phosphorylated Tau (p-tau), for example, p-tau 181, neurofilament light (NfL), and/or any combination thereof. In some embodiments, the CSF inflammatory marker comprises glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, the CSF immune cell chemokine ligand comprises CX3CL1 (Fractalkine). In some embodiments, the CSF innate immune receptor biomarker comprises soluble TREM2. In some embodiments, the CSF biomarker comprises, without limitation, one or more of CD3+/CD56− T cells (e.g., % CD3+/CD56− T cells in leukocytes and/or lymphocytes), % CX3CR1+ cells in CD3−CD56+ NK Cells, and % CX3CR1+ cells in CD3+/CD56− T cells.

In some embodiments, a method of treating Alzheimer's disease in a subject, the method comprising: identifying a subject, wherein the subject has Alzheimer's; and administering to the subject an expanded natural killer (NK) cell population, wherein the NK cells are expanded by a method comprising: isolating at least one of CD56+ cells and/or CD3−/CD56+ cells from the PBMCs; co culturing the at least one of CD56+ cells and/or CD3−/CD56+ cells with a combination of feeder cells in the presence of at least two cytokines; wherein the combination of feeder cells comprises irradiated Jurkat cells and irradiated Epstein Barr virus transformed lymphocyte continuous line (EBV LCL) cells; and wherein the at least two cytokines comprise IL 2 and IL 21, is disclosed.

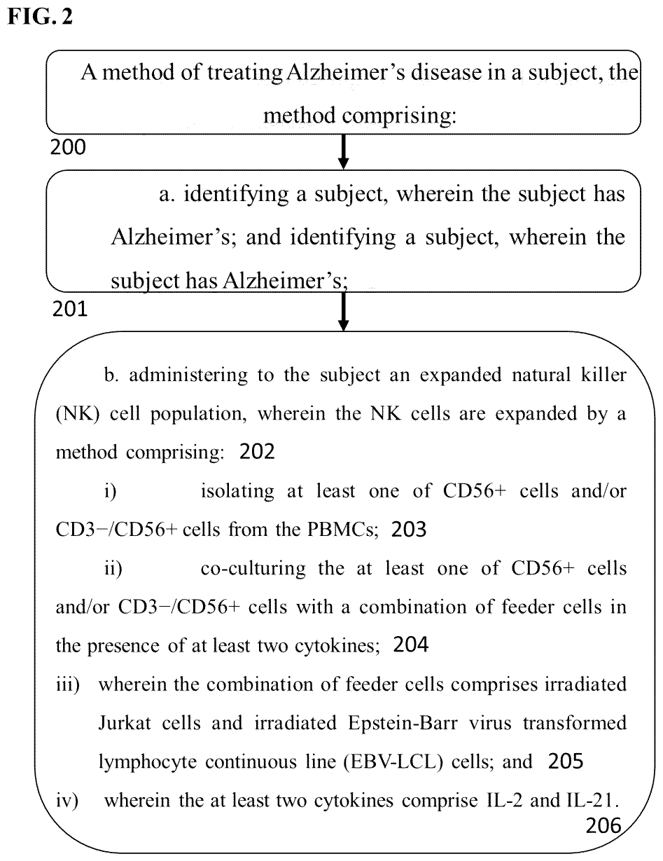

FIG. 2 is a flowchart depicting some embodiments of a method of treating Alzheimer's Disease in a subject.

In some embodiments, a method of treating Alzheimer's disease in a subject is provided 200 (with reference to FIG. 2). In some embodiments, the method comprises: identifying a subject, wherein the subject has Alzheimer's disease, at block 201; and administering to the subject an expanded natural killer (NK) cell population, at block 202. In some embodiments, the NK cells are expanded by a method comprising: i) isolating at least one of CD56+ cells and/or CD3−/CD56+ cells from the PBMCs, at block 203; ii) co-culturing the at least one of CD56+ cells and/or CD3−/CD56+ cells with a combination of feeder cells in the presence of at least two cytokines, at block 204; iii) wherein the combination of feeder cells comprises irradiated Jurkat cells and irradiated Epstein-Barr virus transformed lymphocyte continuous line (EBV-LCL) cells, at block 205; and iv) wherein the at least two cytokines comprise IL-2 and IL-21, at block 206. In some embodiments, the method further comprises administering one or more cognitive assessments to the subject. In some embodiments, the cognitive assessment is administered before and/or after administration of the NK cells. In some embodiments the cognitive assessment comprises CDR, MMSE, ADAS-Cog, and/or any combination thereof. In some embodiments, the method further comprises detecting and/or quantifying one or more biomarkers of AD. In some embodiments, the biomarker is a CSF or plasma biomarker. In some embodiments, the CSF biomarker is a core CSF biomarker, a CSF inflammatory biomarker, a CSF immune cell chemokine ligand, a CSF innate immune receptor, and/or any combination thereof. In some embodiments, the CSF core biomarker comprises amyloid beta 42, amyloid beta 40, amyloid beta 42/40 ratio, total Tau (t-tau), phosphorylated Tau (p-tau), p-tau 181, neurofilament light (NfL), and/or any combination thereof. In some embodiments, the CSF inflammatory marker comprises glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, the CSF immune cell chemokine ligand comprises CX3CL1 (Fractalkine). In some embodiments, the CSF biomarker comprises, without limitation, one or more of CD3+/CD56− T cells (e.g., % CD3+/CD56− T cells in leukocytes and/or lymphocytes), % CX3CR1+ cells in CD3−CD56+ NK Cells, and % CX3CR1+ cells in CD3+/CD56− T cells. In some embodiments, the CSF innate immune receptor biomarker comprises soluble TREM2. In some embodiments, the one or more plasma biomarkers comprise amyloid beta 42, amyloid beta 40, amyloid beta 42/40 ratio, total Tau (t-tau), phosphorylated Tau (p-tau), Glial Fibrillary Acidic Protein (GFAP), and neurofilament light (NfL). Plasma inflammatory markers included YKL-40, IL-1β, IL-6, IL-8, IL-10, TNF-α, and/or INF-γ, or any combination thereof. In some embodiments, the one or more biomarkers is detected and/or quantified before and/or after administration of the NK cells. In some embodiments, administration of the expanded NK cell population results in an improvement in the subject's score on one or more cognitive assessments. In some embodiments the cognitive assessment comprises CDR, MMSE, ADAS-Cog, and/or any combination thereof. In some embodiments, administration of the expanded NK cell population results in a decrease in the subject's CDR score. In some embodiments, administration of the expanded NK cell population results in a decrease in the subject's ADAS-Cog score. In some embodiments, administration of the expanded NK cell population results in an increase in the subject's MMSE score. In some embodiments, administration of the expanded NK cell population results in an increase or decrease in one or more CSF and/or plasma biomarkers of AD. In some embodiments, the one or more biomarkers comprise amyloid beta 42, amyloid beta 40, amyloid beta 42/40 ratio, total Tau (t-tau), phosphorylated Tau (p-tau), p-tau 181, Glial Fibrillary Acidic Protein (GFAP), and neurofilament light (NfL). In some embodiments, the CSF biomarker comprises, without limitation, one or more of CD3+/CD56− T cells (e.g., % CD3+/CD56− T cells in leukocytes and/or lymphocytes), % CX3CR1+ cells in CD3−CD56+ NK Cells, and % CX3CR1+ cells in CD3+/CD56− T cells. Plasma inflammatory markers include YKL-40, IL-1β, IL-6, IL-8, IL-10, TNF-α, and/or INF-γ, or any combination thereof. In some embodiments, administration of the expanded NK cell population results in an increase in amyloid beta 42, amyloid beta 42/40 ratio, and/or IL-8. In some embodiments, administration of the expanded NK cell population results in a decrease in total tau, p-tau, GFAP, NfL, YKL-40, CX3CL1 (Fractalkine), IL-6, TNF-α, IL-12/IL-23p40, and/or sTREM2. In some embodiments, administration of the expanded NK cell population results in decreased neuroinflammation.

In some embodiments, a method of cell therapy comprising: identifying a subject, wherein the subject has Alzheimer's disease; and administering to the subject an expanded NK cell population, wherein the NK cells are expanded by a method comprising: isolating at least one of CD56+ cells and/or CD3−/CD56+ cells from the PBMCs; co culturing at least one of CD56+ cells and/or CD3−/CD56+ cells with a combination of feeder cells in the presence of at least two cytokines; wherein the combination of feeder cells comprises irradiated Jurkat cells and irradiated Epstein Barr virus transformed lymphocyte continuous line (EBV LCL) cells; and wherein at least two cytokines comprise IL 2 and IL 21.

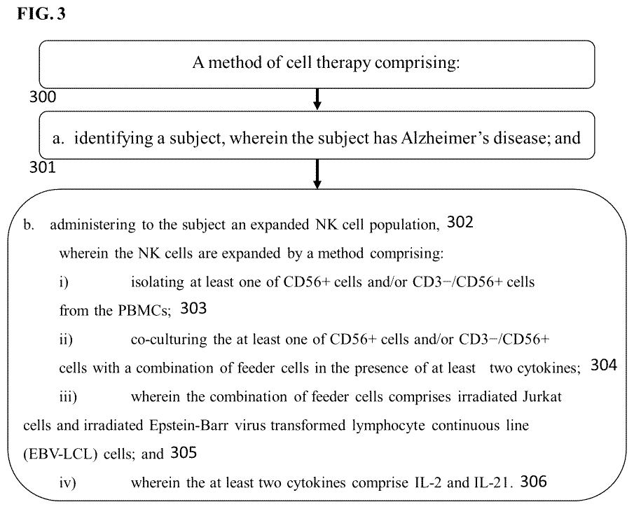

FIG. 3 is a flowchart depicting some embodiments of a method of cell therapy.

In some embodiments, a method of cell therapy is provided 300 (with reference to FIG. 3), comprising: identifying a subject, wherein the subject has Alzheimer's disease, at block 301; and administering to the subject an expanded NK cell population, at block 302. In some embodiments, the NK cells are expanded by a method comprising: i) isolating at least one of CD56+ cells and/or CD3−/CD56+ cells from the PBMCs, at block 303; ii) co-culturing the at least one of CD56+ cells and/or CD3−/CD56+ cells with a combination of feeder cells in the presence of at least two cytokines, at block 304; iii) wherein the combination of feeder cells comprises irradiated Jurkat cells and irradiated Epstein-Barr virus transformed lymphocyte continuous line (EBV-LCL) cells, at block 305; and iv) wherein the at least two cytokines comprise IL-2 and IL-21, at block 306.

In some embodiments, the method further comprises administering one or more cognitive assessments to the subject. In some embodiments, the cognitive assessment is administered before and/or after administration of the NK cells. In some embodiments the cognitive assessment comprises CDR, MMSE, ADAS-Cog, and/or any combination thereof. In some embodiments, the method further comprises detecting and/or quantifying one or more biomarkers of AD. In some embodiments, the biomarker is a CSF or plasma biomarker. In some embodiments, the CSF biomarker is a core CSF biomarker, a CSF inflammatory biomarker, a CSF immune cell chemokine ligand, a CSF innate immune receptor, and/or any combination thereof. In some embodiments, the CSF core biomarker comprises amyloid beta 42, amyloid beta 40, amyloid beta 42/40 ratio, total Tau (t-tau), phosphorylated Tau (p-tau), p-tau 181, neurofilament light (NfL), and/or any combination thereof. In some embodiments, the CSF inflammatory marker comprises glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, the CSF immune cell chemokine ligand comprises CX3CL1 (Fractalkine). In some embodiments, the CSF innate immune receptor biomarker comprises soluble TREM2. In some embodiments, the one or more plasma biomarkers comprise amyloid beta 42, amyloid beta 40, amyloid beta 42/40 ratio, total Tau (t-tau), phosphorylated Tau (p-tau), p-tau 181, Glial Fibrillary Acidic Protein (GFAP), and neurofilament light (NfL). Plasma inflammatory markers included YKL-40, IL-1β, IL-6, IL-8, IL-10, TNF-α, and/or INF-γ, or any combination thereof. In some embodiments, the one or more biomarkers is detected and/or quantified before and/or after administration of the NK cells.

In some embodiments, administration of the expanded NK cell population results in an improvement or stability in the subject's score on one or more cognitive assessments. In some embodiments the cognitive assessment comprises CDR, MMSE, ADAS-Cog, and/or any combination thereof. In some embodiments, administration of the expanded NK cell population results in a decrease in the subject's CDR score or CDR-SB score. In some embodiments, administration of the expanded NK cell population results in a decrease in the subject's ADAS-Cog score. In some embodiments, administration of the expanded NK cell population results in an increase in the subject's MMSE score.

In some embodiments, the change in cognitive function of the subject is assessed against a suitable minimal clinically important difference (MCID) for AD. In some embodiments, the change in cognitive function of the subject is assessed against a suitable minimal clinically important difference (MCID) for mild AD. In some embodiments, the change in CDR-SB score is assessed based on an MCID of at least f 0.5, 1, 1.5, 2, 2.5, or 3, or a difference in a range defined by any two of the preceding values (e.g., ±0.5-3, ±1-2.5, ±1.5-2.5, etc.). In some embodiments, the change in CDR-SB score is assessed based on an MCID of at least, or of at least about f 2. In some embodiments, the change in CDR-SB score is clinically meaningful for mild AD when the CDR-SB score increases by at least, or at least about 2. In some embodiments, the change in CDR-SB score indicates an improved or stable assessment when there is a reduction in CDR-SB, no change in CDR-SB, or an increase in CDR-SB that is not meaningful (e.g., clinically meaningful) for mild AD.

In some embodiments, the change in ADAS-Cog score is assessed based on an MCID of at least f 0.5, 1, 1.5, 2, 2.5, 3, 3.5, 4, 4.5, or 5, or a difference in a range defined by any two of the preceding values (e.g., ±0.5-5, ±1-4.5, ±2-4, etc.). In some embodiments, the change in ADAS-Cog score is assessed based on an MCID of at least, or of at least about ±3. In some embodiments, the change in ADAS-Cog score is clinically meaningful for mild AD when the CDR-SB score increases by at least, or at least about 3. In some embodiments, the change in ADAS-Cog score indicates an improved or stable assessment when there is a reduction in ADAS-Cog, no change in ADAS-Cog, or an increase in ADAS-Cog that is not meaningful (e.g., clinically meaningful) for mild AD.

In some embodiments, the change in MMSE score is assessed based on an MCID of at least 0.5, 1, 1.5, 2, 2.5, or 3, or a difference in a range defined by any two of the preceding values (e.g., ±0.5-3, ±1-2.5, ±1.5-2.5, etc.). In some embodiments, the change in MMSE score is assessed based on an MCID of at least, or of at least about ±2. In some embodiments, the change in MMSE score is clinically meaningful for mild AD when the MMSE score decreases by at least, or at least about 2. In some embodiments, the change in MMSE score indicates an improved or stable assessment when there is an increase in MMSE, no change in MMSE, or a decrease in MMSE that is not meaningful (e.g., clinically meaningful) for mild AD.

In some embodiments, administration of the expanded NK cell population results in an improvement in, or a stable assessment in one or more cognitive assessments over the course of treatment. As used herein, an improvement in or stable cognitive assessment denotes the assessment not showing a change that indicates having or worsening of the disease (e.g., Alzheimer's), as described herein, over the relevant time period (e.g., increased CDR-SB, or ADAS, or reduced MMSE). In some embodiments, an improvement in or stable cognitive assessment is based on an MCID of the cognitive test (e.g., for mild AD). In some embodiments, administration of the expanded NK cell population results in an improvement in, or stable cognitive assessment for Alzheimer's disease (e.g., no increase in CDR-SB, or ADAS, or no reduction in MMSE) in, in about, or in at least 20%, 30%, 40%, 50%, 60%, 70%, 80%, or about 90%, or a percentage in a range defined by any two of the preceding values (e.g., 20-90%, 30-90%, 40-80%, 60-90%, 30-90%, etc.) of the treated subjects, up to about 12 weeks, or more, after the last dose. In some embodiments, administration of the expanded NK cell population results in an improvement in, or stable cognitive assessment for Alzheimer's disease (e.g., no increase in CDR-SB, or ADAS, or no reduction in MMSE) in, in about, or in at least 20%, 30%, 40%, 50%, 60%, 70%, 80%, or about 90%, or a percentage in a range defined by any two of the preceding values (e.g., 20-90%, 30-90%, 40-80%, 60-90%, 30-90%, etc.) of the treated subjects, between 1 week after the last dose and 11 weeks after the last dose. In some embodiments, administration of the expanded NK cell population results in an improvement in, or stable cognitive assessment for Alzheimer's disease (e.g., no increase in CDR-SB, or ADAS, or no reduction in MMSE) in at least about 65% and up to about 85% of the treated subjects, between 1 week after the last dose and 11 weeks after the last dose. In some embodiments, administration of the expanded NK cell population results in an improvement in, or stable cognitive assessment for Alzheimer's disease (e.g., no increase in CDR-SB, or ADAS, or no reduction in MMSE) in at least about 50% and up to about 85% of the treated subjects, at least up to about 12 weeks after the last dose.

In some embodiments, administration of the expanded NK cell population results in an increase or decrease in one or more CSF and/or plasma biomarkers of AD. In some embodiments, the one or more CSF and/or plasma biomarkers comprise amyloid beta 42, amyloid beta 40, amyloid beta 42/40 ratio, total Tau (t-tau), phosphorylated Tau (p-tau), Glial Fibrillary Acidic Protein (GFAP), and neurofilament light (NfL). Plasma inflammatory biomarkers include, without limitation, YKL-40, IL-1β, IL-6, IL-8, IL-10, TNF-α, and/or INF-γ, or any combination thereof. In some embodiments, the CSF biomarker comprises, without limitation, one or more of CD3+CD56− T cells (e.g., % CD3+CD56− T cells in leukocytes and/or lymphocytes), % CX3CR1+ cells in CD3−CD56+ NK Cells, and % CX3CR1+ cells in CD3+/CD56− T cells. In some embodiments, administration of the expanded NK cell population results in an increase in or a stable level of (e.g., lack of a decrease in) amyloid beta 42, amyloid beta 42/40 ratio, and/or IL-8 (e.g., as measured in CSF or plasma). In some embodiments, administration of the expanded NK cell population results in an increase or a stable level of CSF or plasma amyloid beta 42, amyloid beta 42/40 ratio, and/or IL-8 in, in about, or in at least 20%, 30%, 40%, 50%, 60%, 70%, or 80%, or a percentage in a range defined by any two of the preceding values (e.g., 20-80%, 30-80%, 40-70%, 50-80%, etc.) of the treated subjects, up to about 12 weeks, or more, after the last dose. In some embodiments, administration of the expanded NK cell population results in an increase in or a stable level of CSF or plasma amyloid beta 42, amyloid beta 42/40 ratio, and/or IL-8 in, in about, or in at least 20%, 30%, 40%, 50%, 60%, 70%, or 80%, or a percentage in a range defined by any two of the preceding values (e.g., 20-80%, 30-80%, 40-70%, 50-80%, etc.) of the treated subjects, from 1-12 weeks, or more, after the last dose. In some embodiments, administration of the expanded NK cell population results in an increase or a stable level of CSF or plasma amyloid beta 42 and/or amyloid beta 42/40 ratio in at least about 30% and up to about 75% of the treated subjects, from 1-12 weeks or more after the last dose. In some embodiments, administration of the expanded NK cell population results in an increase or a stable level of plasma amyloid beta 42 and/or amyloid beta 42/40 ratio in at least about 50% and up to about 75% of the treated subjects, at least up to about 12 weeks after the last dose.

In some embodiments, administration of the expanded NK cell population results in a decrease in or a stable level of (e.g., lack of an increase in) total tau, p-tau, p-tau 181, GFAP, NfL, YKL-40, CX3CL1 (Fractalkine), IL-6, TNF-α, IL-12/IL-23p40, and/or sTREM2 (e.g., as measured in CSF or plasma). In some embodiments, administration of the expanded NK cell population results in a decrease or a stable level of CSF or plasma p-Tau 181, GFAP, NfL, and/or YKL-40 in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the subjects, up to about 12 weeks, or more, after the last dose. In some embodiments, administration of the expanded NK cell population results in a decrease or a stable level of CSF or plasma p-tau 181, GFAP, NfL, and/or YKL-40 in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the subjects, from 1-12 weeks, or more, after the last dose. In some embodiments, administration of the expanded NK cell population results in a decrease or a stable level of CSF p-tau 181, GFAP, NfL, and/or YKL-40 in at least about 30% and up to about 100% of the treated subjects, from 1-12 weeks or more after the last dose. In some embodiments, administration of the expanded NK cell population results in a decrease or a stable level of plasma p-tau 181, GFAP, NfL, and/or YKL-40 in at least about 50% and up to about 100% of the treated subjects, at least up to about 12 weeks, after the last dose. In some embodiments, administration of the expanded NK cell population results in decreased neuroinflammation.

In methods of treating Alzheimer's disease of the present disclosure, in some embodiments, administration of the expanded NK cell population results in an improvement in, or stable CSF and/or plasma levels of protein biomarkers and/or neuroinflammation markers over the course of treatment. As used herein, an improvement or stable level of a biomarker or neuroinflammation marker denotes the level of the biomarker or neuroinflammation marker not showing a change that is associated with or with worsening of the disease (e.g., Alzheimer's), as described herein, over the relevant time period. In some embodiments, administration of the expanded NK cell population results in an improvement in, or stable CSF and/or plasma levels of one or more protein biomarkers for Alzheimer's disease (e.g., amyloid beta 42, amyloid beta 42/40 ratio, and/or p-tau 181). In some embodiments, administration of the expanded NK cell population results in an improvement, or stable CSF and/or plasma levels of one or more protein biomarkers for Alzheimer's disease (e.g., amyloid beta 42, amyloid beta 42/40 ratio, and/or p-tau 181) in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the treated subjects, up to about 12 weeks, or more, after the last dose. In some embodiments, administration of the expanded NK cell population results in an improvement, or stable CSF and/or plasma levels of one or more protein biomarkers for Alzheimer's disease (e.g., amyloid beta 42, amyloid beta 42/40 ratio, and/or p-tau 181) in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the treated subjects, from 1-12 weeks, or more, after the last dose. In some embodiments, administration of the expanded NK cell population results in an improvement, or stable CSF levels of one or more protein biomarkers for Alzheimer's disease (e.g., amyloid beta 42, amyloid beta 42/40 ratio, and/or p-tau 181) in at least about 30% and up to about 100% of the treated subjects, from 1-12 weeks or more after the last dose. In some embodiments, administration of the expanded NK cell population results in an improvement, or stable plasma levels of one or more protein biomarkers for Alzheimer's disease (e.g., amyloid beta 42, amyloid beta 42/40 ratio, and/or p-tau 181) in at least about 50% and up to about 100% of the treated subjects, at least up to about 12 weeks after the last dose.

In any method of treating Alzheimer's disease of the present disclosure, in some embodiments, administration of the expanded NK cell population results in an improvement in, or stable CSF and/or plasma levels of one or more neuroinflammation markers (e.g., GFAP, NfL, and/or YKL-40). In some embodiments, administration of the expanded NK cell population results in an improvement, or stable CSF and/or plasma levels of one or more neuroinflammation markers (e.g., GFAP, NfL, and/or YKL-40) in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the treated subjects, up to about 12 weeks, or more, after the last dose. In some embodiments, administration of the expanded NK cell population results in an improvement, or stable CSF and/or plasma levels of one or more neuroinflammation markers (e.g., GFAP, NfL, and/or YKL-40) in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the treated subjects, from 1-12 weeks, or more, after the last dose. In some embodiments, administration of the expanded NK cell population results in an improvement, or stable CSF levels of one or more neuroinflammation markers (e.g., GFAP, NfL, and/or YKL-40) in at least about 30% and up to about 90% of the treated subjects, from 1-12 weeks or more after the last dose. In some embodiments, administration of the expanded NK cell population results in an improvement, or stable plasma levels of one or more neuroinflammation markers (e.g., GFAP, NfL, and/or YKL-40) in at least about 50% and up to about 75% of the treated subjects, at least up to about 12 weeks, after the last dose.

In any method of treating Alzheimer's disease of the present disclosure, in some embodiments, a subject shows rebound from an improvement (e.g., reversing or halting an improvement) in, or from stable CSF and/or plasma levels of one or more neuroinflammation markers (e.g., GFAP, NfL, and/or YKL-40) after administration of the expanded NK cell population is terminated. In some embodiments, rebound from an improvement or stable CSF levels of one or more neuroinflammation markers (e.g., GFAP, NfL, and/or YKL-40) after administration of the expanded NK cell population is terminated is observed in, in about, or in at least 20%, 30%, 40%, 50%, 60%, 70%, 80%, 90%, or about 100%, or a percentage in a range defined by any two of the preceding values (e.g., 20-100%, 30-90%, 40-80%, 60-100%, 30-100%, etc.) of the treated subjects. In some embodiments, rebound from an improvement or stable CSF levels of one or more neuroinflammation markers (e.g., GFAP, NfL, and/or YKL-40) after administration of the expanded NK cell population is terminated is observed in at least about 20% and up to about 100% of the treated subjects.

In any method of treating Alzheimer's disease of the present disclosure, in some embodiments, a subject shows rebound from an improvement in, or stable CSF and/or plasma levels of one or more protein biomarkers for Alzheimer's disease (e.g., amyloid beta 42, amyloid beta 42/40 ratio, and/or p-tau 181) after administration of the expanded NK cell population is terminated. In some embodiments, rebound from an improvement or stable CSF levels of one or more protein biomarkers for Alzheimer's disease (e.g., amyloid beta 42, amyloid beta 42/40 ratio, and/or p-tau 181) after administration of the expanded NK cell population is terminated is observed in, in about, or in at least 20%, 30%, 40%, 50%, 60%, or about 70%, or a percentage in a range defined by any two of the preceding values (e.g., 20-70%, 30-60%, 40-60%, etc.) of the treated subjects. In some embodiments, rebound from an improvement or stable CSF levels of one or more protein biomarkers for Alzheimer's disease (e.g., amyloid beta 42, amyloid beta 42/40 ratio, and/or p-tau 181) after administration of the expanded NK cell population is terminated is observed in at least about 25% and up to about 60% of the treated subjects.

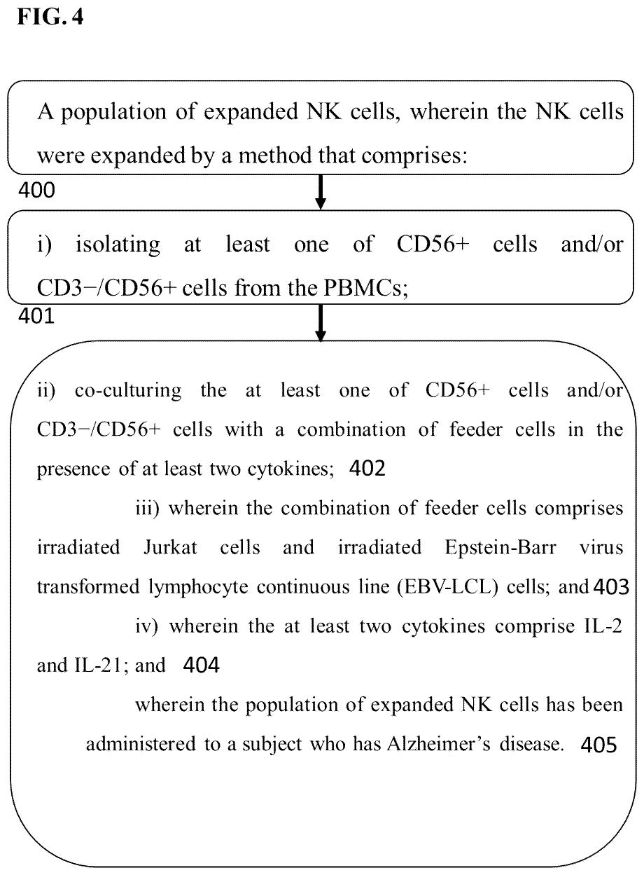

FIG. 4 is a flowchart depicting some embodiments of a population of expanded NK cells.

In some embodiments, a population of expanded NK cells 400 is provided (with reference to FIG. 4). In some embodiments, the NK cells were expanded by a method that comprises: i) isolating at least one of CD56+ cells and/or CD3−/CD56+ cells from the PBMCs, at block 401; ii) co-culturing the at least one of CD56+ cells and/or CD3−/CD56+ cells with a combination of feeder cells in the presence of at least two cytokines, at block 402; iii) wherein the combination of feeder cells comprises irradiated Jurkat cells and irradiated Epstein-Barr virus transformed lymphocyte continuous line (EBV-LCL) cells, at block 403; and iv) wherein the at least two cytokines comprise IL-2 and IL-21, at block 404. In some embodiments, the population of expanded NK cells has been administered to a subject who has Alzheimer's disease. In some embodiments, administration of the NK cells improves the subject's performance on one or more cognitive assessments. In some embodiments, the cognitive assessment is administered before and/or after administration of the NK cells. In some embodiments the cognitive assessment comprises CDR, MMSE, ADAS-Cog, and/or any combination thereof. In some embodiments, administration of the NK cells alters the levels of one or more biomarkers of AD. In some embodiments, the biomarker is a CSF or plasma biomarker. In some embodiments, the CSF biomarker is a core CSF biomarker, a CSF inflammatory biomarker, a CSF immune cell chemokine ligand, a CSF innate immune receptor, and/or any combination thereof. In some embodiments, the CSF core biomarker comprises amyloid beta 42, amyloid beta 40, amyloid beta 42/40 ratio, total Tau (t-tau), phosphorylated Tau (p-tau), p-tau 181, neurofilament light (NfL), and/or any combination thereof. In some embodiments, the CSF inflammatory marker comprises glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, the CSF immune cell chemokine ligand comprises CX3CL1 (Fractalkine). In some embodiments, the CSF innate immune receptor biomarker comprises soluble TREM2. In some embodiments, the one or more plasma biomarkers comprise amyloid beta 42, amyloid beta 40, amyloid beta 42/40 ratio, total Tau(t-tau), phosphorylated Tau (p-tau), p-tau 181, Glial Fibrillary Acidic Protein (GFAP), and neurofilament light (NfL). Plasma inflammatory markers included YKL-40, IL-1β, IL-6, IL-8, IL-10, TNF-α, and/or INF-γ, or any combination thereof. In some embodiments, the one or more biomarkers is detected and/or quantified before and/or after administration of the NK cells. In some embodiments, administration of the expanded NK cell population results in an improvement in the subject's score on one or more cognitive assessments. In some embodiments the cognitive assessment comprises CDR, MMSE, ADAS-Cog, and/or any combination thereof. In some embodiments, administration of the expanded NK cell population results in a decrease in the subject's CDR score. In some embodiments, administration of the expanded NK cell population results in a decrease in the subject's ADAS-Cog score. In some embodiments, administration of the expanded NK cell population results in an increase in the subject's MMSE score. In some embodiments, administration of the expanded NK cell population results in an increase or decrease in one or more CSF and/or plasma biomarkers of AD. In some embodiments, the one or more biomarkers comprise amyloid beta 42, amyloid beta 40, amyloid beta 42/40 ratio, total Tau (t-tau), phosphorylated Tau (p-tau), p-tau 181, Glial Fibrillary Acidic Protein (GFAP), and neurofilament light (NfL). Plasma inflammatory markers include YKL-40, IL-1β, IL-6, IL-8, IL-10, TNF-α, and/or INF-γ, or any combination thereof. In some embodiments, administration of the expanded NK cell population results in an increase in amyloid beta 42, amyloid beta 42/40 ratio, and/or IL-8. In some embodiments, administration of the expanded NK cell population results in a decrease in total tau, p-tau, p-tau 181, GFAP, NfL, YKL-40, CX3CL1 (Fractalkine), IL-6, TNF-α, IL-12/IL-23p40, and/or sTREM2. In some embodiments, administration of the expanded NK cell population results in decreased neuroinflammation.

In some embodiments, a method of treating Alzheimer's disease in a subject, the method comprising: identifying a subject, wherein the subject has Alzheimer's disease; and administering to the subject a therapeutically effective amount of an autologous NK cell population (or an allogeneic NK cell population), is disclosed.

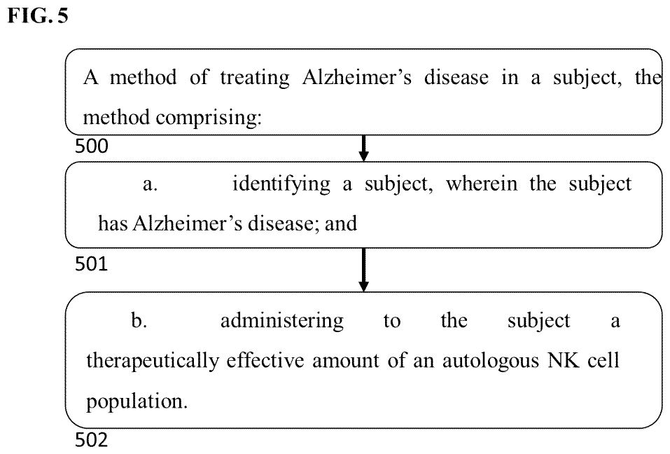

FIG. 5 is a flowchart depicting some embodiments of a method of treating Alzheimer's Disease in a subject.

In some embodiments, a method of treating Alzheimer's disease in a subject 500 is provided (with reference to FIG. 5). In some embodiments, the method comprises identifying a subject, wherein the subject has Alzheimer's, at block 501; and administering to the subject a therapeutically effective amount of an autologous natural killer cell (NK) cell population, at block 502 (or an allogeneic NK cell population). In some embodiments, the method further comprises administering one or more cognitive assessments to the subject. In some embodiments, the cognitive assessment is administered before and/or after administration of the NK cells. In some embodiments the cognitive assessment comprises CDR, MMSE, ADAS-Cog, and/or any combination thereof. In some embodiments, the method further comprises detecting and/or quantifying one or more biomarkers of AD. In some embodiments, the biomarker is a CSF or plasma biomarker. In some embodiments, the CSF biomarker is a core CSF biomarker, a CSF inflammatory biomarker, a CSF immune cell chemokine ligand, a CSF innate immune receptor, and/or any combination thereof. In some embodiments, the CSF core biomarker comprises amyloid beta 42, amyloid beta 40, amyloid beta 42/40 ratio, total Tau (t-tau), phosphorylated Tau (p-tau), neurofilament light (NfL), and/or any combination thereof. In some embodiments, the CSF inflammatory marker comprises glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, the CSF immune cell chemokine ligand comprises CX3CL1 (Fractalkine). In some embodiments, the CSF innate immune receptor biomarker comprises soluble TREM2. In some embodiments, the one or more plasma biomarkers comprise amyloid beta 42, amyloid beta 40, amyloid beta 42/40 ratio, total Tau (t-tau), phosphorylated Tau (p-tau), Glial Fibrillary Acidic Protein (GFAP), and neurofilament light (NfL). Plasma inflammatory markers included YKL-40, IL-1β, IL-6, IL-8, IL-10, TNF-α, and/or INF-γ, or any combination thereof. In some embodiments, the one or more biomarkers is detected and/or quantified before and/or after administration of the NK cells. In some embodiments, administration of the expanded NK cell population results in an improvement in the subject's score on one or more cognitive assessments. In some embodiments the cognitive assessment comprises CDR, MMSE, ADAS-Cog, and/or any combination thereof. In some embodiments, administration of the expanded NK cell population results in a decrease in the subject's CDR score. In some embodiments, administration of the expanded NK cell population results in a decrease in the subject's ADAS-Cog score. In some embodiments, administration of the expanded NK cell population results in an increase in the subject's MMSE score. In some embodiments, administration of the expanded NK cell population results in an increase or decrease in one or more CSF and/or plasma biomarkers of AD. In some embodiments, the one or more biomarkers comprise amyloid beta 42, amyloid beta 40, amyloid beta 42/40 ratio, total Tau (t-tau), phosphorylated Tau (p-tau), Glial Fibrillary Acidic Protein (GFAP), and neurofilament light (NfL). Plasma inflammatory markers included YKL-40, IL-1β, IL-6, IL-8, IL-10, TNF-α, and/or INF-γ, or any combination thereof. In some embodiments, administration of the expanded NK cell population results in an increase in amyloid beta 42, amyloid beta 42/40 ratio, and/or IL-8. In some embodiments, administration of the expanded NK cell population results in a decrease in total tau, p-tau, p-tau 181GFAP, NfL, YKL-40, CX3CL1 (Fractalkine), IL-6, TNF-α, IL-12/IL-23p40, and/or sTREM2. In some embodiments, administration of the expanded NK cell population results in decreased neuroinflammation.

In some embodiments, the amount of expanded NK cells administered to a subject is a therapeutically effective amount.

In some embodiments, the therapeutically effective amount of expanded NK cells comprises 0.1×10⁹ to 12×10⁹ cells. In some embodiments, the amount is 0.1×10⁹, 0.5×10⁹, 1×10⁹, 2×10⁹, 3×10⁹, 4×10⁹, 5×10⁹, 6×10⁹, 7×10⁹, 8×10⁹, 9×10⁹, 10×10⁹, 11×10⁹, 12×10⁹, or more. In some embodiments, the therapeutically effective amount of expanded NK cells comprises 0.1×10⁹-1×10¹⁴ cells, 0.5×10⁹-1×10¹¹ cells, 1×10⁹-1×10¹⁰ cells, 1×10⁹-1×10¹¹ cells, or 1×10⁹-5×10¹⁰ cells. In some embodiments it is any one of the preceding amounts given every 1, 2, 3, 4, 5, 6, 7, or 8 weeks. In some embodiments, it is 3-5 billion cells given every 2-4 weeks (e.g., 4 billion cells every 3 weeks). In any method provided herein, in some embodiments, the maximum number of NK cells administered is 9×10⁹ cells. In any method provided herein, in some embodiments, the maximum number of NK cells administered is 1×10¹² cells.

In some embodiments, at least 1, 2, 3, 4, 5, 6, 7, 8, 9, 10, 11, 12, 13, 14, 15, 16, 17, 18, 19, 20 or more doses of expanded NK cells is administered to the subject.

In some embodiments, IL-2 is added at a concentration of 50-1000 IU/mL during step ii).

In some embodiments IL-21 is added at a concentration of 10-100 ng/mL during step ii).

In some embodiments, the Mini-Mental State Exam (MMSE) score of the subject is between 20-25 after treatment with expanded NK cells.

In some embodiments, the Mini-Mental State Exam (MMSE) score of the subject is ≥25 after treatment with expanded NK cells.

In some embodiments, expansion of NK cells further comprises: co-culturing the at least one of CD56+ cells and/or CD3−/CD56+ cells with the combination of feeder cells, in the presence of IL-2 for a first period; and co-culturing the at least one of CD56+ cells and/or CD3−/CD56+ cells with the combination of feeder cells, in the presence of IL-21 for a second period.

In some embodiments, IL-21 is added more than once during Day 0-6 of the second period.

In some embodiments, IL-21 and the combination of feeder cells are added more than once during Day 0-6 of the second period.

In some embodiments, IL-21 is added more than once during the first six days of every fourteen-day cycle during the second period.

In some embodiments, the NK cells do not include a chimeric antigen receptor (CAR).

In some embodiments, the NK cells do not include an engineered CAR.

In any method of the present disclosure, in some embodiments, the NK cells to be administered can be NK cells that have been expanded with any suitable option for expanding NK cells. In some embodiments, the NK cells are autologous (e.g., autologous to the subject to which the NK cells are administered). In some embodiments, the NK cells are or comprise SNK01. (See www(dot)sec(dot)gov/ix?doc=/Archives/edgar/data/1845459/000110465923074785/gfor-20230331xs4a.htm, which is incorporated by reference herein as to SNK01). As used herein, “SNK01” denotes SNK01 autologous NK cells produced by NKGen Biotech, Inc. (Irvine, CA). In some embodiments, the NK cells are or comprise SNK01 autologous cells, produced by NKGen Biotech, Inc. (Irvine, CA). Suitable options for expanding NK cells are provided in, e.g., PCT publication No. WO 2019/152663, which is incorporated by reference in its entirety herein. In some embodiments, the NK cells are allogeneic (e.g., allogeneic to the subject to which the NK cells are administered).

In some embodiments, any of the above steps can have further steps added between them. In some embodiments, any one or more of the above steps can be performed concurrently or out of the order provided herein.

In some embodiments, administration of NK cells improves the subject's score on one or more cognitive assessments. In some embodiments, the cognitive assessment is a CDR In some embodiments, administration of the NK cells decreases the subjects CDR by about 0.1, 0.2, 0.3, 0.4, 0.5, 0.6, 0.7, 0.8, 0.9, 1, 1.5, 2, or 2.5 points, or by a range that is defined by any two of the preceding values. For example, in some embodiments, the subject's CDR score is decreased by about 0.1-2.5, 0.1-2, 0.1-1.5, 0.1-1, 0.1-0.5, 0.5-2.5, 0.5-2, 0.5-1.5, 0.5-1, 1-2.5, 1-2, 1-1.5, 1.5-2.5, 1.5-2, or 2-2.5, points following one or more administrations of the NK cells.

In some embodiments, administration of NK cells improves the subject's score on one or more cognitive assessments. In some embodiments, the cognitive assessment is a MMSE. In some embodiments, administration of the NK cells increases the subjects MMSE score by about 1, 2, 3, 4, 5, 6, 7, 8, 9, 10, 11, 12, 12, 14, or 15, points, or by a range that is defined by any two of the preceding values. For example, in some embodiments, the subject's MMSE score is increased by about 1-15, 1-10, 1-7, 1-5, 1-3, 3-15, 3-10, 3-7, 3-5, 5-15, 5-10, 5-7, 7-15 7-10, or 10-15, points following one or more administrations of the NK cells.

In some embodiments, administration of NK cells improves the subject's score on one or more cognitive assessments. In some embodiments, the cognitive assessment is an ADAS-Cog. In some embodiments, administration of the NK cells decreases the subjects ADAS-Cog by about 1, 2, 3, 4, 5, 6, 7, 8, 9, 10, 15, 20, 25, 30, 35, 40, 35, 50, 55, 60, 65, or 70 points, or by a range that is defined by any two of the preceding values. For example, in some embodiments, the subject's ADAS-Cog score is decreased by between about 1-70, 1-50, 1-25, 1-10, 1-7, 1-5, 1-3, 3-70, 3-50, 3-25, 3-10, 3-7, 3-5, 5-70, 5-50, 5-25, 5-10, 5-7, 7-70, 7-50, 7-25, 7-10, 10-70, 10-50, 10-25, 25-70, 25-50, or 50-70, points following one or more administrations of the NK cells.

In some embodiments, the level of one or more CSF and/or plasma biomarkers increases following one or more administrations of the NK cells. In some embodiments, the biomarker is amyloid beta 42. In some embodiments, the level of amyloid beta 42 in the subject's CSF increases by about 1, 2, 3, 4, 5, 6, 7, 8, 9, 10, 20, 25, 30, 40, 50, 60, 70, 75, 80, 90, 100, 150, 200, 250, 300, 400, or 500%, or by an amount in a range that is defined by any two of the preceding values, following one or more administrations of the NK cells. For example, in some embodiments, the level of amyloid beta 42 in the subject's CSF increases by between about 1-500, 1-250, 1-100, 1-75, 1-50, 1-25, 1-10, 10-500, 10-250, 10-100, 10-75, 10-50, 10-25, 25-500, 25-250, 25-100, 25-75, 25-50, 50-500, 50-250, 50-100, 50-75, 75-500, 75-250, 75-100, 100-500, 100-250, or 250-500%, following one or more administrations of the NK cells. In some embodiments, the level of amyloid beta 42 in the subject's CSF increases by about 28-275% following one or more administrations of the NK cells.

In some embodiments, the level of one or more CSF and/or plasma biomarkers increases following one or more administrations of the NK cells. In some embodiments, the biomarker is amyloid beta 42/40 ratio. In some embodiments, the ratio of amyloid beta 42 to amyloid beta 40 in the subject's CSF increases by about 1, 2, 3, 4, 5, 6, 7, 8, 9, 10, 20, 25, 30, 40, 50, 60, 70, 75, 80, 90, 100, 150, 200, 250, 300, 400, or 500%, or by an amount in a range that is defined by any two of the preceding values, following one or more administrations of the NK cells. For example, in some embodiments, the ratio of amyloid beta 42 amyloid beta 40 in the subject's CSF increases by between about 1-500, 1-250, 1-100, 1-75, 1-50, 1-25, 1-10, 10-500, 10-250, 10-100, 10-75, 10-50, 10-25, 25-500, 25-250, 25-100, 25-75, 25-50, 50-500, 50-250, 50-100, 50-75, 75-500, 75-250, 75-100, 100-500, 100-250, or 250-500%, following one or more administrations of the NK cells. In some embodiments, the ratio of amyloid beta 42 amyloid beta 40 in the subject's CSF increases by between about 40-264% following one or more administrations of the NK cells.

In some embodiments, the level of one or more CSF and/or plasma biomarkers increases following one or more administrations of the NK cells. In some embodiments, the biomarker is IL-8. In some embodiments, the level of IL-8 in the subject's CSF increases by about 1, 2, 3, 4, 5, 6, 7, 8, 9, 10, 20, 25, 30, 40, 50, 60, 70, 75, 80, 90, 100, 150, 200, 250, 300, 400, or 500%, or by an amount in a range that is defined by any two of the preceding values, following one or more administrations of the NK cells. For example, in some embodiments, the level of IL-8 in the subject's CSF increases by between about 1-500, 1-250, 1-100, 1-75, 1-50, 1-25, 1-10, 10-500, 10-250, 10-100, 10-75, 10-50, 10-25, 25-500, 25-250, 25-100, 25-75, 25-50, 50-500, 50-250, 50-100, 50-75, 75-500, 75-250, 75-100, 100-500, 100-250, or 250-500%, following one or more administrations of the NK cells. In some embodiments, the level of IL-8 in the subject's CSF increases by between about 25-108% following one or more administrations of the NK cells.

In some embodiments, the level of one or more CSF and/or plasma biomarkers decreases following one or more administrations of the NK cells. In some embodiments, the biomarker is total tau. In some embodiments, the level of total tau in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of total tau in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of total tau in the subject's CSF decreases by between about 21-84% following one or more administrations of the NK cells.

In some embodiments, the level of one or more CSF and/or plasma biomarkers decreases following one or more administrations of the NK cells. In some embodiments, the biomarker is p-tau. In some embodiments, the level of p-tau in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of p-tau in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of p-tau in the subject's CSF decreases by about 9-94% following one or more administrations of the NK cells.

In some embodiments, the level of one or more CSF and/or plasma biomarkers decreases following one or more administrations of the NK cells. In some embodiments, the biomarker is p-tau 181. In some embodiments, the level of p-tau 181 in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of p-tau 181 in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of p-tau 181 in the subject's CSF decreases by about 9-94% following one or more administrations of the NK cells.

In some embodiments, the level of one or more CSF and/or plasma biomarkers decreases following one or more administrations of the NK cells. In some embodiments, the biomarker is GFAP. In some embodiments, the level of GFAP in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of GFAP in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of GFAP in the subject's CSF decreases by between about 36-95% following one or more administrations of the NK cells.

In some embodiments, the level of one or more CSF and/or plasma biomarkers decreases following one or more administrations of the NK cells. In some embodiments, the biomarker is NfL. In some embodiments, the level of NfL in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of NfL in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. in some embodiments, the level of NfL in the subject's CSF decreases by between about 4-71% following one or more administrations of the NK cells.

In some embodiments, the level of one or more CSF and/or plasma biomarkers decreases following one or more administrations of the NK cells. In some embodiments, the biomarker is YKL-40. In some embodiments, the level of YKL-40 in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of YKL-40 in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. in some embodiments, the level of YKL-40 in the subject's CSF decreases by between about 4-73% following one or more administrations of the NK cells.

In some embodiments, the level of one or more CSF and/or plasma biomarkers increases following one or more administrations of the NK cells. In some embodiments, the biomarker is CX3CL1 (Fractalkine). In some embodiments, the level of CX3CL1 (Fractalkine) in the subject's CSF increases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of CX3CL1 (Fractalkine) in the subject's CSF increases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of CX3CL1 (Fractalkine) in the subject's CSF increases by between about 18-231% following one or more administrations of the NK cells.

In some embodiments, the level of one or more CSF and/or plasma biomarkers increases following one or more administrations of the NK cells. In some embodiments, the biomarker is % CX3CR1+ cells in CD3−CD56+ NK cells. In some embodiments, the % CX3CR1+ cells in CD3−CD56+ NK cells in the subject's CSF increases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the % CX3CR1+ cells in CD3−CD56+ NK cells in the subject's CSF increases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells.

In some embodiments, the level of one or more CSF and/or plasma biomarkers is changed following one or more administrations of the NK cells. In some embodiments, the biomarker is % CD3+CD56− T cells in lymphocytes and/or leukocytes. In some embodiments, the % CD3+CD56− T cells in lymphocytes and/or leukocytes in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the % CD3+CD56− T cells in lymphocytes and/or leukocytes in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells.

In some embodiments, the level of one or more CSF and/or plasma biomarkers decreases following one or more administrations of the NK cells. In some embodiments, the biomarker is IL-6. In some embodiments, the level of IL-6 in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of IL-6 in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of IL-6 in the subject's CSF decreases by between about 19-65% following one or more administrations of the NK cells.

In some embodiments, the level of one or more CSF and/or plasma biomarkers decreases following one or more administrations of the NK cells. In some embodiments, the biomarker is TNF-α. In some embodiments, the level of TNF-α in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of TNF-α in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of TNF-α in the subject's CSF decreases by between about 42-96% following one or more administrations of the NK cells.

In some embodiments, the level of one or more CSF and/or plasma biomarkers decreases following one or more administrations of the NK cells. In some embodiments, the biomarker is IL-12/IL-23p40 ratio. In some embodiments, the ratio of IL-12 to IL-23p40 in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the ratio of IL-12 to IL-23p40 in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. in some embodiments, the ratio of IL-12 to IL-23p40 in the subject's CSF decreases by between about 7-53% following one or more administrations of the NK cells.

In some embodiments, the level of one or more CSF and/or plasma biomarkers decreases following one or more administrations of the NK cells. In some embodiments, the biomarker is sTREM2. In some embodiments, the level of sTREM2 in the subject's CSF decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the level of sTREM2 in the subject's CSF decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. In some embodiments, the level of sTREM2 in the subject's CSF decreases by between about 26-73% following one or more administrations of the NK cells.

In some embodiments, the subject's level of neuroinflammation decreases following one or more administrations of the NK cells (e.g., the expanded NK cells). In some embodiments, the subject's level of neuroinflammation decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the subject's level of neuroinflammation decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. The level of neuroinflammation can be measured using any suitable option. In some embodiments, the level of neuroinflammation is measured by assaying the level or change in level of one or more markers of inflammation in plasma or CSF, e.g., as described herein.

A method for producing high-purity NK cells without using expensive cytokines has been developed. After CD56+ cells are isolated from peripheral blood mononuclear cells, when the CD56+ cells isolated from peripheral blood mononuclear cells are co-cultured with feeder cells in the presence of cytokines, high-purity CD56+ NK cells could be produced. Also, a cell therapeutic composition for treating Alzheimer's disease comprising NK cells which are effectively usable for autologous therapy is provided herein. As a result, when a specific cytokine was added to CD56+ NK cells isolated from peripheral blood mononuclear cells, high survival rate and high activity were exhibited. Therefore, in some embodiments, the treatment of Alzheimer's disease involves or includes a method for expanding NK cells and to provide a cell therapeutic composition for the treatment of Alzheimer's disease comprising expanded peripheral blood-derived CD56+ NK cells. In some embodiments, the NK cells are effectively usable for allogeneic therapy.

According to some embodiments, a method for producing high-purity NK cells may include: isolating peripheral blood mononuclear cells (PBMCs) from a blood sample (“First Isolation Step”); isolating cells selected from a group consisting of CD56+ cells and CD3−/CD56+ cells from the peripheral blood mononuclear cells (“Second Isolation Step”); and co-culturing the cells selected from a group consisting of CD56+ cells and CD3−/CD56+ cells together with feeder cells in the presence of cytokine (“Culturing Step”). Each step is described in greater detail herein. The CD3−/CD56+ cells produced according to the disclosed method may exhibit not only higher purity and higher activity, but also other distinguished characteristics, such as having different surface markers or activated receptors, for example, one or more from CD16, CD25, CD27, CD28, CD69, CD94/NKG2C, CD94/NKG2E, CD266, CD244, NKG2D, KIR2S, KIR3S, Ly94D, NCRs, IFN-a, IFN-b, CXCR3, CXCR4, CX3CR1, CD62L and CD57.

### First Isolation Step

In the present specification, the “blood sample” may be, but not limited to, whole blood of the peripheral blood or leukocytes isolated from the peripheral blood using leukapheresis. Further, the peripheral blood may be obtained from a healthy person, a patient having a risk of Alzheimer's disease, or an Alzheimer's patient, but the source of the peripheral blood is not limited thereto.

In the present specification, the term “leukapheresis” may refer to a method of selectively collecting leukocytes from the collected blood and then giving the blood to a patient again, and in some embodiments, the leukocytes isolated by the method may be used without additional methods such as a Ficoll-Hypaque density gradient method.

In the present specification, the term “peripheral blood mononuclear cell” may be used interchangeably with “PBMC”, “mononuclear cell”, and may refer to a mononuclear cell isolated from the peripheral blood. The peripheral blood mononuclear cells may be obtained from the collected human blood using known methods such as a Ficoll-Hypaque density gradient method.

In some embodiments, the peripheral blood mononuclear cells may be autologous, but allogenic peripheral blood mononuclear cells may also be used for producing high-purity NK cells for immunotherapy according to methods described herein. Further, in some embodiments, the peripheral blood mononuclear cells may be obtained from a healthy person, but the peripheral blood mononuclear cells may be also obtained from a patient having a risk of Alzheimer's disease and/or an Alzheimer's patient.

In the present specification, the term “CD56+ cells” may be used interchangeably with “CD56+ NK cells”, or “CD56+ natural killer cells”, and the term “CD3−/CD56+ cells” may be used interchangeably with “CD3−/CD56+ NK cells.” The CD56+ cells or CD3−/CD56+ cells may include cells in which CD56 glycoprotein on the cell surface is expressed, or further, cells in which CD3 glycoprotein is not expressed while the CD56 glycoprotein is expressed. Even the same type of immune cells may have differences in CD type attached to the cell surface and expression rate and thus, the functions thereof may be different.

### Second Isolation Step

In some embodiments, the isolating of the CD56+ natural killer cells from the blood sample may be performed by an isolating method using at least one selected from the group consisting of CD56 microbeads and CD3 microbeads, or an isolating method using equipment such as CliniMACSs, a flow cytometry cell sorter, etc.

For example, the isolating method using the CD56 microbeads and/or the CD3 microbeads may be performed by adding the CD56 microbeads to PBMCs and then removing non-specific binding. Isolation of CD3−/CD56+ cells may be performed by adding CD3 microbeads to the PBMCs to deplete CD3+ cells then adding the CD56 microbeads again to enrich the CD56+ cells. In some instances, through isolating CD56+ cells and/or CD3−/CD56+ cells from PBMCs, T cells or other non-natural killer cells may be removed.

### Culturing Step

In the present specification, the term “cytokine” may refer to an immunoactive compound that is capable of inducing the peripheral blood mononuclear cells to differentiate into NK cells.

In some embodiments, the cytokine may be interleukin-2 (IL-2), IL-15, IL-21, FMS-like tyrosine kinase 3 ligand (Flt3-L), a stem cell factor (SCF), IL-7, IL-18, IL-4, type I interferons, a granulocyte-macrophage colony-stimulating factor (GM-CSF), and an insulin-like growth factor 1 (IGF 1), but not limited thereto.

In some embodiments, the cytokine may be used at a concentration of 50-1,000, 50-900, 50-800, 50-700, 50-600, 50-550, 100-550, 150-550, 200-550, 250-550, 300-550, 350-550, 400-550, 450-550 IU/mL. Conventional methods of proliferating NK cells utilize high concentrations of various cytokines. Conversely, in some embodiments of the method of proliferating NK cells described herein, since two types of feeder cells may be used with the high-purity CD56+ cells, NK cells with high yield and high purity may be proliferated using only low concentrations of one cytokine.

In the present specification, the term “feeder cell” may refer to a cell of which proliferation is blocked by gamma irradiation, but has metabolic activity to produce various metabolites and thus, helps the proliferation of target cells.

In some embodiments, the feeder cells may be at least one selected from the group consisting of irradiated Jurkat cells, irradiated Epstein-Barr virus transformed lymphocyte continuous line (EBV-LCL) cells, and PBMC, HFWT, RPMI 1866, Daudi, MM-170, K562 or cells genetically modified by targeting K562 (for example, K562-mbIL-15-41BB ligand). For example, in one embodiment, the feeder cells may be the irradiated Jurkat cells and the EBV-LCL cells.

In the present specification, the term “Jurkat cell” or “Jurkat cell line” may refer to a blood cancer (immortalized acute T cell leukemia) cell line, which has been developed by Dr. Arthur Weiss of the University of California at San Francisco. Jurkat cells, in which various chemokine receptors are expressed and capable of producing IL-2, have not generally been considered as a possible candidate of the feeder cells for immunotherapy because MHC class I, which is a natural killer cell activation inhibitor, is highly expressed on the cell surface thereof. The Jurkat cells may be obtained from the ATCC (ATCC TIB-152).

In the present specification, the term “EBV-LCL cell” or “EBV-LCL cell line” refers to an Epstein-Barr virus transformed lymphocyte continuous line (EBV-LCL) (D. M. Koelle et al., J Clin Invest, 1993: 91: 961-968), which is a B cell line that is made by infecting human B cells with Epstein-Barr virus in a test tube. The EBV-LCL cells may be directly prepared and used in a general laboratory by a method of adding cyclosporine A in a process of infecting EBV in the PBMC. In some embodiments, the EBV-LCL cell may be prepared by following steps. 30×10⁶ PBMCs are added in 9 mL of a culture medium, the mixture is added in a T 25 culture flask, and then 9 mL of an EBV supernatant is added. 80 μL of cyclosporine A (50 μg/mL) is added and then cultured at 37° C. After 7 days of culture, a half of supernatant is removed, a fresh culture medium is added, and then 40 μL of cyclosporine A is added. The same process may be repeated once every 7 days until 28 days of culture. The cell line may be usable after 28 days of culture, and from this time, the cell line may be cultured in the culture medium without adding cyclosporine A.

The Jurkat cells and the EBV-LCL cells may be used as the feeder cells after irradiation. In some embodiments, the irradiated Jurkat cells and the irradiated EBV-LCL cells may be included at a content ratio of 1:0.1-5, 1:0.1-4, 1:0.1-3, 1:0.1-2, 1:0.1-1.5, 1:0.5-1.5, 1:0.75-1.25, 0.1-5:1, 0.1-4:1, 0.1-3:1, 0.1-2:1, 0.1-1.5:1, 0.5-1.5:1 or 0.75-1.25:1. For example, the irradiated Jurkat cells and the irradiated EBV-LCL cells may be included at a content ratio of 1:1.

In some embodiments, the irradiated Jurkat cells and the irradiated EBV-LCL cells may be obtained by treating with irradiation of 50-500, 50-400, 50-300, 50-200, 50-150, 70-130, 80-120 or 90-110 Gy. For example, the irradiated Jurkat cells and/or the irradiated EBV-LCL cells may be obtained by treating Jurkat cells and/or EBV-LCL cells with irradiation of 100 Gy.

In some embodiments, the culturing may be performed for 1-50, 1-42, 1-40, 1-35, 1-20, 1-19, 1-18, 1-17, 1-16, 1-15 or 1-14 days.

In some embodiments, the culturing step may further include following steps: co-culturing with the feeder cells and a first cytokine (“first culturing step”); and further co-culturing after addition of a second cytokine (“second culturing step”)

The second culturing step may include adding the second cytokine once or more between day 0-6 of culturing. For example, the second culturing step may include adding the second cytokine once on each of day 0 and day 3 of culturing.

The second culturing step may include adding the second cytokine and the feeder cells during the first 6 days of the cycle of 14 days of culturing. For example, the second culturing step may include adding the feeder cells during a 14 days cycle, and adding the second cytokine on day 3 and 6 of each cycle once each.

In some embodiments, the first cytokine may be IL-2. In some embodiments, the second cytokine may be IL-21. In some embodiments, the second cytokine may be used at the concentration of 10-1000, 10-500, 10-100, 20-100, 30-100, 40-100, 50-100 or 10-50 ng/mL. In some embodiments, culturing with the addition of the second cytokine once or more during day 0-6 may exhibit superior proliferation and/or activity. In some embodiments, culturing with the addition of the feeder cells and the second cytokine for six days in the cycle of 14 days may exhibit superior proliferation and/or activity.

In some embodiments, the co-culturing may be performed by including the peripheral blood mononuclear cells and the feeder cells (for example, the Jurkat cells and the EBV-LCL cells) at a mixing ratio of 1:1-100, 1:1-90, 1:1-80, 1:1-70, 1:10-65, 1:20-65, 1:30-65, 1:40-65, 1:50-65 or 1:55-65.

The co-culturing may be performed in a medium and any suitable media generally used for induction and proliferation of the peripheral blood mononuclear cells to the NK cells in the art may be used without a limitation as such a medium. For example, an RPMI-1640, DMEM, x-vivo10, x-vivo20, or cellgro SCGM medium may be used as such a medium. In addition, the culture conditions such as a temperature may follow any suitable culture conditions of the peripheral blood mononuclear cells known in the art.

In some embodiments, within the produced NK cells, purity of the CD56+ NK cells may be 85% or more, 90% or more, or 95% or more, or 98% or more with respect to the whole cells. In some embodiments, within the produced NK cells, a ratio of T cells to whole cells may be 15% or less, 10% or less, 5% or less, 2% or less, 1% or less.

In some embodiments, the cytokines IL-2 and IL-21 are capable of supporting expansion of a CD3−/CD56+, or CD56+ population in vitro. In some embodiments, the population of CD3−/CD56+ or CD56+ cells expanded with IL-2 and IL-21 possesses an NK cell phenotype.

In some embodiments, the method of treatment of Alzheimer's disease involves culturing and/or expanding cells in line with one or more of the approaches outlined in U.S. Pat. No. 10,590,385.

In the present specification, the term “peripheral blood-derived” may mean that the cells are derived from “whole blood of the peripheral blood” or “leukocytes isolated from the peripheral blood using leukapheresis.” The peripheral blood derived CD56+ NK cells may be used interchangeably with peripheral blood mononuclear cell (PBMC) derived CD56+ NK cells.

In some embodiments, the term “subject” refers to a mammal which is a subject for treatment, observation, or testing, and preferably, a human. The subject may be a patient of Alzheimer's disease, but not limited thereto.

In some embodiments, in the case of an adult, the cell therapeutic composition may be administered once to several times a day. The cell therapeutic composition may be administered every day or in a 2-180 day interval. the cell therapeutic agent included in the composition may include 1×10⁶ to 1×10¹¹ peripheral blood-derived CD56+ natural killer cells, for example, about 1×10⁶ to 1×10⁸ NK cells per kg of body weight. In some preferred embodiments, the cell therapeutic agent included in the composition may include 2×10⁹ to 9×10⁹ peripheral blood-derived CD56+ natural killer cells. In some embodiments, the peripheral blood-derived CD56+ natural killer cells in the cell therapeutic composition are at least about 90% pure. In some embodiments, the cytokine is IL-2 at a concentration ranging from about 50-50,000 IU/ml.

In some embodiments, the cell therapeutic composition of the present invention may be administered by any suitable method, such as administration through a rectal, intravenous, intraarterial, intraperitoneal, intramuscular, intrasternal, percutaneous, topical, intraocular, or intradermal route. In some embodiments, the NK cells included in the composition may be allogeneic, i.e. obtained from a person other than the subject being treated. In some embodiments, the person may be a healthy person or a patient with Alzheimer's disease. In some embodiments, the NK cells included in the composition may be autologous, i.e. obtained from the subject being treated. In some embodiments, the NK cells included in the composition may be allogeneic, e.g., obtained from a healthy donor other than the subject being treated.

In some embodiments the subject has Alzheimer's disease. Alzheimer's is a neurodegenerative disorder that affects the central nervous system (CNS). Alzheimer's is characterized by the buildup of amyloid-beta plaques and neurofibrillary tangles in the brain. Hallmarks of Alzheimer's disease include motor deficits such as difficulty moving, and cognitive problems including depression, agitation and dementia.

In some embodiments, identifying a subject with Alzheimer's disease comprises a medical diagnosis of Alzheimer's disease. In some embodiments, diagnosis of Alzheimer's disease comprises assessment of memory impairment, thinking skills, and behavioral changes. In some embodiments, diagnosis of Alzheimer's disease comprises imaging via CT, MRI, PET, and/or DaT scan.

Natural killer cells (NK cells) are one type of innate immune cells, which are known to recognize and kill virus-infected and tumor cells by releasing cytotoxic granules such as perforin and granzyme or by death receptor-mediated cytotoxicity.

In some embodiments the NK cells administered to the patient are autologous to the subject. In some embodiments, the NK cells administered to the patient are allogeneic with respect to the subject. In some embodiments, the NK cells administered are derived from a healthy subject. In some embodiments, the NK cells administered are derived from a subject, with disease such as a subject with Alzheimer's.

In some embodiments, the NK cell population has undergone expansion prior to administration. In some embodiments, an autologous NK cell population was expanded in vitro prior to administration. In some embodiments, an allogeneic NK cell population was expanded in vitro prior to administration. In some embodiments NK cell expansion is accomplished by feeder cells. In some embodiments, NK cell expansion is accomplished by cytokine stimulation. In some embodiments, NK cell expansion is accomplished by both cytokines and feeder cells. In some embodiments, expansion of NK cells results in a population with a high purity of NK cells.

In some embodiments, the ratio of CD56+ NK cells to whole cells (purity) may be 85% or more, 90% or more, 95% or more, or 98% or more.

In some embodiments, the composition may not include T cells, or may include only trace amount of T cells. For example, the ratio of T cells to whole cells in the composition may be less than 15%, less than 10%, less than 5%, less than 2%, less than 1% or less.

In some embodiments, the NK cells are co-administered with a cytokine. In some embodiments the cytokine is IL-2, IL-21, IL-15, Flt3-L, IL-7, SCF, IL-18, IL-4, type I IFN, GM-CSF, IGF I, or any combinations thereof. In some embodiments, the cytokine may be used at a concentration of 18-180,000, 20-100,000, 50-50,000, 50-1,000, 50-900, 50-800, 50-700, 50-600, 50-550, 100-550, 150-550, 200-550, 250-550, 300-550, 350-550, 400-550, 450-550 IU/mL. When the cytokine is used in these ranges, it may suppress apoptosis of the NK cells included in the treatment composition and increase activity of the NK cells.

In the present specification, the term “cell therapeutic agent” refers to a medicine which is used for treatment, diagnosis, and prevention through a series of actions, such as proliferating and screening autologous, allogeneic, and xenogenic living cells in vitro for restoring functions of cells and tissues or changing biological characteristics of the cells by other methods. The cell therapeutic agents have been regulated as medical products from 1993 in the USA and 2002 in Korea. These cell therapeutic agents may be largely classified into two fields, that are, first, stem cell therapeutic agents for tissue regeneration or recovery of organ functions, and second, immune cell therapeutic agents for regulation of immune responses, such as inhibition of the immune response or enhancement of the immune response in vivo.

The cell therapeutic composition described herein may be formulated in a suitable form together with a pharmaceutically acceptable carrier suitable or generally used for cell therapy. The “pharmaceutically acceptable” refers to a composition which is physiologically acceptable and does not generally cause an allergic reaction such as gastrointestinal disorders, dizziness, or the like, or similar reactions thereto, when being administered to the human body. The pharmaceutically acceptable carrier may include, for example, parenteral administration carriers such as water, suitable oils, saline, aqueous glucose and glycol, and the like, and further include stabilizers and preservatives. The suitable stabilizer includes an antioxidant such as sodium hydrogen sulfite, sodium sulfite, or ascorbic acid, sucrose, albumin, or the like. The suitable preservative includes DMSO, glycerol, ethylene glycol, sucrose, trehalose, dextrose, polyvinylpyrrolidone, or the like.

The cell therapeutic composition may also be administered by any device in which the cell therapeutic agent may move to the target cell.

In some embodiments, the method comprises administering the doses about 1, 2, 3, 4, 5, 6, 7, 8, 9, 10, 11, 12 weeks apart, including ranges between any two of the listed values. For example, in some embodiments, the method comprises administering the doses between about 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12, weeks apart. In some embodiments, cognitive and motor functions of the patient is monitored at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and/or 52, weeks, including ranges between any two of the listed values. For example, in some embodiments, cognitive and motor functions of the patient is monitored at between about 1-52, 1-40, 1-30, 1-20, 1-10, 1-8, 1-4, 4-52, 4-40, 4-30, 4-20, 4-10, 4-8, 8-52, 8-40, 8-30, 8-20, 8-10, 10-52, 10-40, 10-30, 10-20, 20-52, 20-48, 20-44, 20-40, 20-30, 30-52, 30-40, or 40-52, weeks. In some embodiments, after 1, 2, 3, 4, 5, 6, 7, 8, 9, 10, 11, 12, 13, 14, 15, 16, 17, 18, 19, 20, 21, 22, 23, or 24 months, or after a number of months in a range defined by any two of the preceding values, the NK cell-treated group will exhibit improved cognitive and motor functions. For example, in some embodiments, after between about 1-24, 1-18, 1-12, 1-8, 1-4, 4-24, 4-18, 4-12, 4-8, 8-24, 8-18, 8-12, 12-14, 12-18, or 18-24, months the NK cell-treated group will exhibit improved cognitive and motor functions. In some embodiments, the NK cell-treated group will exhibit improved cognitive and motor functions in greater than about 24 months.

In some embodiments, the method comprises administering the doses in combination with one or more Alzheimer's disease therapies (“secondary therapies”).

In some embodiments, the secondary Alzheimer's disease therapy comprises a physical procedure.

In some embodiments, the secondary Alzheimer's disease therapy comprises administration of one or more therapeutics. In some embodiments, the secondary Alzheimer's disease therapeutic comprises aducanumab.

In some embodiments the method comprises administering doses of the one or more secondary Alzheimer's disease therapeutics about 1, 2, 3, 4, 5, 6, 7, 8, 9, 10, 11, 12 weeks apart, including ranges between any two of the listed values. For example, in some embodiments, the method comprises administering of the one or more secondary Alzheimer's disease therapeutics between about 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12, weeks apart. In some embodiments, cognitive and motor functions of the patient are monitored at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and/or 52, weeks, including ranges between any two of the listed values. For example, in some embodiments, cognitive and motor functions of the patient is monitored at between about 1-52, 1-40, 1-30, 1-20, 1-10, 1-8, 1-4, 4-52, 4-40, 4-30, 4-20, 4-10, 4-8, 8-52, 8-40, 8-30, 8-20, 8-10, 10-52, 10-40, 10-30, 10-20, 20-52, 20-48, 20-44, 20-40, 20-30, 30-52, 30-40, or 40-52, weeks. In some embodiments, after 1, 2, 3, 4, 5, 6, 7, 8, 9, 10, 11, 12, 13, 14, 15, 16, 17, 18, 19, 20, 21, 22, 23, or 24 months, or after a number of months in a range defined by any two of the preceding values, the NK cell-secondary therapeutic treated group will exhibit improved cognitive and motor functions. For example, in some embodiments, after between about 1-24, 1-18, 1-12, 1-8, 1-4, 4-24, 4-18, 4-12, 4-8, 8-24, 8-18, 8-12, 12-14, 12-18, or 18-24, months the NK cell-secondary therapeutic treated group will exhibit improved cognitive and motor functions. In some embodiments, the NK cell-secondary therapeutic treated group will exhibit improved cognitive and motor functions in greater than about 24 months.

In some embodiments the method comprises administering doses of the one or more secondary Alzheimer's disease therapeutics about 1, 2, 3, 4, 5, 6, 7, 8, 9, 10, 11, 12 weeks apart from administration of NK cells, including ranges between any two of the listed values. For example, in some embodiments, the method comprises administering of the one or more secondary Alzheimer's disease therapeutics between about 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12, weeks apart from administration of NK cells. In some embodiments, cognitive and motor functions of the patient will be monitored at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and/or 52, weeks, including ranges between any two of the listed values. For example, in some embodiments, cognitive and motor functions of the patient is monitored at between about 1-52, 1-40, 1-30, 1-20, 1-10, 1-8, 1-4, 4-52, 4-40, 4-30, 4-20, 4-10, 4-8, 8-52, 8-40, 8-30, 8-20, 8-10, 10-52, 10-40, 10-30, 10-20, 20-52, 20-48, 20-44, 20-40, 20-30, 30-52, 30-40, or 40-52, weeks. In some embodiments, after 1, 2, 3, 4, 5, 6, 7, 8, 9, 10, 11, 12, 13, 14, 15, 16, 17, 18, 19, 20, 21, 22, 23, or 24 months, or after a number of months in a range defined by any two of the preceding values, the NK cell-secondary therapeutic treated group will exhibit improved cognitive and motor functions. For example, in some embodiments, after between about 1-24, 1-18, 1-12, 1-8, 1-4, 4-24, 4-18, 4-12, 4-8, 8-24, 8-18, 8-12, 12-14, 12-18, or 18-24, months the NK cell-secondary therapeutic treated group will exhibit improved cognitive and motor functions. In some embodiments, the NK cell-secondary therapeutic treated group will exhibit improved cognitive and motor functions in greater than about 24 months. In some embodiments the NK cells and secondary Alzheimer's disease therapeutic are administered simultaneously. In some embodiments the NK cells and secondary Alzheimer's disease therapeutic are co-administered.

In some embodiments, the NK cells are administered with more than one secondary Alzheimer's disease therapeutics. In some embodiments, the NK cells are administered with 1, 2, 3, 4, 5, 6, 7, 8, 9, or 10, or a range that is defined by any two of the preceding values, secondary Alzheimer's disease therapies and or therapeutics. For example, in some embodiments, the NC cells are administered with between 1-10, 1-7. 1-5, 1-3, 3-10, 3-7, 3-5, 5-10, 5-7, or 7-10, secondary Alzheimer's disease therapies and or therapeutics.

In some embodiments, the NK cells are administered prior to administration of the one or more secondary Alzheimer's disease therapies or therapeutics. For example, in some embodiments, about 1, 2, 3, 4, 5, 6, 7, 8, 9, 10, 11, 12, 13, 14, 15, 16, 17, 18, 19, or 20 doses, or a range of doses that is defined by any two of the preceding values, of the NK cells are administered prior to administration of the one or more secondary Alzheimer's disease therapies or therapeutics. For example, in some embodiments, between about 1-20, 1-15, 1-10, 1-5, 5-20, 5-15, 5-10, 10-20, 10-15, or 15-20, doses of NK cells are administered prior to administration of the one or more secondary Alzheimer's disease therapies or therapeutics. In some embodiments, the one or more secondary Alzheimer's disease therapies or therapeutics is administered prior to administration of the NK cells. For example, in some embodiments, about 1, 2, 3, 4, 5, 6, 7, 8, 9, 10, 11, 12, 13, 14, 15, 16, 17, 18, 19, or 20 doses, or a range of doses that is defined by any two of the preceding values, of the one or more secondary Alzheimer's disease therapies or therapeutics is administered prior to administration of the NK cells. For example, in some embodiments, between about 1-20, 1-15, 1-10, 1-5, 5-20, 5-15, 5-10, 10-20, 10-15, or 15-20, doses of the one or more secondary Alzheimer's disease therapies or therapeutics are administered prior to administration of the NK cells. In some embodiments the NK cells and the one or more secondary Alzheimer's disease therapeutics are administered in alternating cycles. For example, in some embodiments, a first cycle of NK cell administration of about 1, 2, 3, 4, 5, 6, 7, 8, 9, 10, 11, 12, 13, 14, 15, 16, 17, 18, 19, or 20 doses of NK cells, or a range of doses that is defined by any two of the preceding values, is followed by a first cycle of secondary Alzheimer's disease therapeutic administration comprising about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, doses. For example, in some embodiments, a first cycle of NK cell administration of between about 1-20, 1-15, 1-10, 1-5, 5-20, 5-15, 5-10, 10-20, 10-15, or 15-20, doses of NK cells is followed by a first cycle of secondary Alzheimer's disease therapeutic administration comprising between about 1-30, 1-25, 1-20, 1-15, 1-10, 1-5, 5-30, 5-25, 5-20, 5-15, 5-10, 10-30, 10-25, 10-20, 10-15, 15-30, 15-25, 15-20, 20-30, 20-25, or 25-30, doses. In some embodiments, the NK cells and the one or more secondary Alzheimer's disease therapeutics are administered for about 1, 2, 3, 4, 5, 6, 7, 8, 9, 10, 11, 12, 13, 14, 15, 16, 17, 18, 19 or 20 cycles, or by a range that is defined by any two of the preceding values. For example, in some embodiments, the NK cells and the one or more secondary Alzheimer's disease therapeutics are administered for between about 1-20, 1-15, 1-10, 1-5, 5-20, 5-15, 5-10, 10-20, 10-15, or 15-20, cycles. In some embodiments, the first cycle of the one or more secondary Alzheimer's disease therapeutics can be administered prior to the first cycle of NK cell administration. In some embodiments, the first cycle of NK cells can be administered prior to the first cycle of administration of the one or more secondary Alzheimer's disease therapeutics. In some embodiments the cyclic administration of NK cells and one or more secondary Alzheimer's disease therapeutics occur simultaneously. In some embodiments, one or more cycles are staggered by about 1, 2, 3, 4, 5, 6, 7, 8, 9, 10, 11, 12, 13, 14, 15, 16, 17, 18, 19, 20, 21, 22, 23, or 24 hours, or by a range that is defined by any two of the preceding values. For example, in some embodiments, one or more cycles are staggered by between about 1-24, 1-18, 1-12, 1-6, 6-24, 6-18, 6-12, 12-24, 12-28, or 18-24, hours. In some embodiments, one or more cycles are staggered by about 1, 2, 3, 4, 5, 6, 7, 10, 14, 15, 20, 21, 25, 28, 29, 30 or 31 days, or by a range that is defined by any two of the preceding values. For example, in some embodiments, one or more cycles are staggered by between about 1-31, 1-30, 1-29, 1-28, 1-20, 1-10, 1-5, 5-31, 5-30, 5-29, 5-28, 5-20, 5-10, 5-15, 15-31, 15-30, 15-29, 15-28, 15-20, 20-31, 20-30, 20-29, 20-28, or 28-31, days. In some embodiments, one or more cycles are staggered by about 1, 2, 3, 4, 5, 6, 7, 8, 9, 10, 11, or 12 months, or by a range that is defined by any two of the preceding values. For example, in some embodiments, one or more cycles are staggered by between about 1-12, 1-8, 1-4, 1-3, 3-12, 3-9, 3-6, 6-12, 6-9, 8-12, or 9-12, months.

In some embodiments, administration of the NK cells in combination with one or more secondary Alzheimer's therapies and/or therapeutics, reduces the amount of NK cells required to achieve a therapeutic effect. For example, in some embodiments, administration of the NK cells in combination with one or more secondary Alzheimer's therapies and/or therapeutics, reduces the dose of NK cells required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the dose of NK cells required to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the dose of NK cells required to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the dose of NK cells required to achieve a therapeutic effect by between about 1-10, 1-7, 1-5, 1-3, 3-10, 3-7, 3-5, 5-10, 5-7, or 7-10-fold.

In some embodiments, administration of the NK cells in combination with one or more secondary Alzheimer's therapies and/or therapeutics, reduces the amount of the one or more secondary Alzheimer's disease therapeutics required to achieve a therapeutic effect. For example, in some embodiments, administration of the NK cells in combination with one or more secondary Alzheimer's therapies and/or therapeutics, reduces the dose of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the dose of the one or more secondary Alzheimer's disease therapeutics required to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the number of doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by between about 1-10, 1-7, 1-5, 1-3, 3-10, 3-7, 3-5, 5-10, 5-7, or 7-10-fold.

In some embodiments, administration of the NK cells in combination with one or more secondary Alzheimer's therapies and/or therapeutics, reduces the amount of NK cells required to achieve a therapeutic effect. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the number of doses of the one or more Alzheimer's disease therapeutics required to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the number of doses of the one or more Alzheimer's disease therapeutics required to achieve a therapeutic effect by between about 1-10, 1-7, 1-5, 1-3, 3-10, 3-7, 3-5, 5-10, 5-7, or 7-10-fold.

In some embodiments, administration of the NK cells in combination with one or more Alzheimer's therapies and/or therapeutics, reduces the amount of the one or more secondary Alzheimer's disease therapeutics required to achieve a therapeutic effect. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the number of doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the number of doses of the one or more secondary Alzheimer's disease therapeutics required to achieve a therapeutic effect by between about 1-10, 1-7, 1-5, 1-3, 3-10, 3-7, 3-5, 5-10, 5-7, or 7-10-fold.

In some embodiments, administration of the NK cells in combination with one or more Alzheimer's therapies and/or therapeutics, reduces the time required for NK cells to achieve a therapeutic effect. For example, in some embodiments, administration of the NK cells in combination with one or more secondary Alzheimer's therapies and/or therapeutics, reduces the time required for NK cells to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the time required for NK cells to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the time required to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the time required to achieve a therapeutic effect by between about 1-10, 1-7, 1-5, 1-3, 3-10, 3-7, 3-5, 5-10, 5-7, or 7-10-fold.

In some embodiments, administration of the NK cells in combination with one or more secondary Alzheimer's therapies and/or therapeutics, reduces the time required for the one or more secondary Alzheimer's disease therapeutics to achieve a therapeutic effect. For example, in some embodiments, administration of the NK cells in combination with one or more secondary Alzheimer's therapies and/or therapeutics, reduces the time required for the one or more secondary Alzheimer's disease therapeutics to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%, or by a range that is defined by any two of the preceding values. For example, in some embodiments, administration of the NK cells in combination with one or more secondary Alzheimer's therapies and/or therapeutics, reduces the time required for the one or more secondary Alzheimer's disease therapeutics to achieve a therapeutic effect by between about 5-99, 5-95, 5-90, 5-75, 5-50, 5-25, 5-10, 10-99, 10-95, 10-90, 10-75, 10-50, 10-25, 25-99, 25-95, 25-90, 25-75, 25-50, 50-99, 50-95, 50-90, 50-75, 75-99, 75-95, 75-90, 90-99, 90-95, or 95-99%. In some embodiments, administration of the NK cells in combination with one or more secondary Alzheimer's therapies and/or therapeutics, reduces the time required for the one or more secondary Alzheimer's disease therapeutics to achieve a therapeutic effect by 1-fold, 2-fold, 3-fold, 4-fold, 5-fold, 6-fold, 7-fold, 8-fold, 9-fold, or 10-fold, or by a range that is defined by any two of the preceding values. For example, in some embodiments, administration of the NK cells in combination with one or more secondary Alzheimer's therapies and/or therapeutics, reduces the time required for the one or more secondary Alzheimer's disease therapeutics to achieve a therapeutic effect by between about 1-10, 1-7, 1-5, 1-3, 3-10, 3-7, 3-5, 5-10, 5-7, or 7-10-fold.

### Pharmaceutical Formulations

A pharmaceutical formulation for treating a disease as described herein can comprise NK cells described herein. In some embodiments, the NK cells can be formulated for systemic administration. In some embodiments, the NK cells can be formulated for parenteral administration. In some embodiments, NK cells are formulated as a pharmaceutical composition for administration to a subject by, but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for systemic administration. In other instances, the pharmaceutical composition describe herein is formulated for oral administration. In still other embodiments, the pharmaceutical composition describe herein is formulated for intranasal administration.

In some embodiments, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel*; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulation can further comprise an additional therapeutic agent. Non-limiting examples of additional therapeutic agents include secondary Alzheimer's disease therapeutics. In some embodiments, the secondary Alzheimer's disease therapeutic is aducanumab, lecanemab, and/or donaneman. In some embodiments, the secondary Alzheimer's disease therapeutic is aducanumab, AAB-003, PF-05236812, AADvac1, Axon peptide 108 conjugated to KLH, ABT 418, ABT-089, ABT-288, ABT-384, ABT-957, ABvac 40, ACI-24, Pal1-15 acetate salt, ACI-3024, Tau Morphomer™, ACI-35, VAC20121, ACU193, ACU-193, AF 102B, cevimeline HCL, Evoxac™, AL002, AL003, AL101, GSK-4527226, ALZ-101, ALZ-801, valiltramiprosate, NRM-8499, homotaurine prodrug, 3-APS, ALZT-OP1, Cromolyn sodium, Intal, Ibuprofen, AN-1792, AIP 001, APNmAb005, RAA7, AR1001, ASN51, ASN121151, AVP-786, AVP-923, Nuedexta, Zenvia, AXS-05, Dextromethorphan/bupropion, AZD1446, TC-6683, AZD3480, ispronicline, TC-1734, AZP2006, AZP-2006, Acetyl-l-carnitine HCl, ALCAR, Acitretin, Soriatane, Neotigason, RO 101670, Aduhelm, Aducanumab, BIIB037, Affitope AD02, Albrioza, AMX0035, Allopregnanolone, brexanolone, 3α-hydroxy-5α-pregnan-20-one, 3α,5α-tetrahydroprogesterone, Alpha-Tocopherol, Vitamin E, Alzhemed™, Vivimind™, Tramiprosate, NC-531, homotaurine, 3-APS, milomotide, CAD106, Aripiprazole, Abilify, BMS-337039, Atabecestat, JNJ-54861911, BACE inhibitor, Atomoxetine, ATX, Strattera, Atorvastatin, Lipitor™, Zarator®, Sortis®, Tahor®, Atuzaginstat, COR388, Avagacestat, BMS-708163, Azeliragon, PF-04494700, TTP488, BI 1181181, VTP 37948, BACE inhibitor, BI 409306, SUB 166499, BI 425809, BIIB076, NI-105, 6C5 huIgG1/1, BIIB080, IONIS-MAPTRx, ISIS 814907, BIIB118, PF-05251749, BPN14770, Bapineuzumab, AAB-001, Baricitinib, Olumiant®, NCB28050, LY3009104, Bepranemab, UCB0107, UCB 0107, Antibody D, Besipirdine HCl HP 749, Bexarotene, Targretin®, Blarcamesine, Anavex 2-73, Bosutinib, BOSULIF®, PF-5208763, SKI-606, Brexpiprazole, Rexulti, OPC34712, Bryostatin 1, CERE-110, Nerve Growth Factor gene therapy, CHF 5074, COGNIShunt™, CX516, Ampalex, Candesartan, Atacand, Candesartan cilexetil, Cannabidiol, CBD, Epidiolex, Carvedilol, Coreg, Artist, Aucardic, Dilatrend, Kredex, Celecoxib, Celebrex, Cerebrolysin, Circadin, Citalopram, escitalopram, Celexa, Lexapro, Cipralex, Clioquinol, iodochlorhydroxyquin, PBT-1, Continuous Positive Airway Pressure, CPAP, Contraloid, PRI-002, contraloid acetate, RD2, Crenezumab MABT5102A, RG7412, Curcumin, diferuloylmethane, Longvida™, DAYVIGO, Lemborexant, E2006, DNL747, SAR 443060, Dapagliflozin, Farxiga, Forxiga, BMS 512148, Dapsone, Avlosulfon, Diaminodiphenylsulfone, DDS, Dasatinib+Quercetin, Deep Brain Stimulation-fomix, Deferiprone, Ferriprox, Dexpramipexole, R-pramipexole, RPPX, KNS-760704, BIIB 050, Dimebon, Dimebolin, Latrepirdine, Pf-01913539, Docosahexaenoic acid (DHA), Omega 3 fatty acid, Donanemab, N3pG-Aβ Monoclonal Antibody, LY3002813, Donepezil Aricept™, Donepezil hydrochloride, Eranz®, E 2020, Dronabinol, THC, Marinol, Syndros, delta-9-tetrahydrocannabinol, delta-9-THC, E2814, EHT 0202, Etazolate, ELND005, AZD-103, Scyllo-inositol, cyclohexane-1,2,3,4,5,6-hexol, EVP-0962, EVP 0015962, Edicotinib, JNJ-40346527, JNJ-527, PRV-6527, Edonerpic, T-817 MA, T 817, Elayta, CT1812, Elenbecestat, E2609, BACE inhibitor, Empagliflozin, Jardiance, BI-10773, Encenicline, EVP-6124, MT-4666, α7-nAChR agonist, Epigallocatechin Gallate (EGCG), Sunphenon EGCg, Epothilone D, BMS-241027, Eptastigmine, MF 201, Estrogen, Premarin™, Etanercept, Enbrel™, Exenatide, Exendin-4, Byetta, Bydureon, Flurizan™, tarenflurbil, r-flurbiprofen, MPC-7869, Fosgonimeton, ATH-1017, NDX-1017, G-CSF, Filgrastim, GC 021109, GENUS, Gamma entrainment using sensory stimuli, GammaSense Stimulation, GLN-1062, Memogain, GRF6019, GSK239512, GSK2647544, GSK-2647544, GSK933776, GV-971, sodium oligomannate, sodium oligo-mannurarate, GV1001, RIAVAX™, Tertomotide, Galantamine, Razadyne™, Reminyl™, Nivalin®, Gammagard®, Intravenous Immunoglobulin, IVIg, Gamunex, Human Albumin Combined With Flebogamma, Gantenerumab, RO4909832, RG1450, Gemfibrozil, Lopid, Jezil, Gen-Fibro, Gosuranemab, BIIB092, BMS-986168, IPN007, Guanfacine, Intuniv, SPD503, Afken, Estulic, Tenex, HF0220, HTL0018318, Huperzine A, ZT-1, DEBIO 9902, Qian ceng ta, Cerebra capsule, Pharmassure Memorall capsule, Advil™, Nuprin™, Motrin™, Idalopirdine, Lu AE58054, SGS 518, Idebenone, Catena, Sovrima, Intepirdine, RVT-101, SB 742457, GSK 742457, Inzomelid, JNJ-63733657, KarXT, xanomeline-trospium, Ketasyn, Axona, Caprylic Acid, AC-1202, LM11A-31-BHS, LM11A-31, LMTM, TRx0237, LMT-X, Methylene Blue, Tau aggregation inhibitor (TAI), LU25-109, LX1001, AAVrh.10-APOE2, AAVrh.10hAPOE2, LY2599666, LY2886721, BACE inhibitor, LY3202626, BACE Inhibitor, LY3372689, LY3372993, N3pG-Abeta mAb, Ladostigil, Ladostigil hemitartrate, TV3326, Lanabecestat, AZD3293, LY3314814, BACE inhibitor, Lecanemab, BAN2401, mAb158, Lenalidomide, Revlimid, Leuprolide, Leuprolide acetate, Lupron Depot, Eligard, Levetiracetam, Keppra, Linopirdine, DuP996, Liraglutide, Victoza™, Saxenda™, Lomecel-B, mesenchymal stem cells, Lomoxicam, Losartan, Cozaar®, MK0954, Lu AF20513, Lu AF87908, Lumateperone ITI-007, MEDI1814, MEM 1003, BAY Z 4406, MK-7622, MKC-231, MSDC-0160, Mitoglitazone, MW150, MW01-18-150SRM, MW151, MW01-2-151SRM, Minozac, MW01-2-151WH, compound 17, Masitinib, Masivet, Kinavet, AB1010, Masitinib mesylate, Masupirdine, SUVN-502, Melatonin, Memantine, Ebixa™, Namenda™ Axura®, Akatinol®, Memary®, Metformin, Glucophage, Glucophage XR, Methylphenidate, Ritalin, Concerta, Metrifonate, trichlorfon, Milameline, CI 979, Minocycline, Solodyn, Arestin, Minocin, Dynacin, Montelukast, Singulair, MK0476, NGX267, AF267B, NIC5-15, Pinitol, D-Pinitol, NS2330, Tesofensine, Nabilone, Cesamet, Naproxen, Aleve™, Anaprox™, Naprosyn™, Nasal Insulin, Detemir, Levemir, Humulin, Novolin, glulisine, Nefiracetam, Neflamapimod, VX-745, Nelonicline, ABT-126, NeoTrofin, AIT-082, leteprinim, Neramexane, MRZ 2/579, Nicotinamide Riboside, Niagen, NR, Nicotinamide, Vitamin B3, Nilotinib, Tasigna, AMN107, Nilvadipine Nilvad, Nivadil, ARC029, Nuplazid, Pimavanserin, ACP-103, Pimavanserin tartrate, ORM-12741, Octagam®10%, NewGam, PBT2, PBT-2, PF-05212377, PF-5212377, WYE-103760, SAM-760, PF-06648671, PF-06751979, BACE inhibitor, PF-06852231, PNT001, PRX-03140, Potassium salt, PU-AD, PU-HZ151, icapamespib, PXT864, PXT00864, Pepinemab, VX15, VX15/2503, Phenserine, Physostigmine Salicylate, Synapton, Piromelatine, Neu-P11, Ponezumab, PF-04360365, Posiphen, ANVS-401, Posiphen tartrate, Prazosin, Prazosin hydrochloride, Minipress, Hypovase, Vasoflex, Prednisone, Propentofylline, HWA 285, PPF, Protollin, RG3487, RO5313534, MEM 3454, RG7129, RO5508887, BACE Inhibitor, RG7345, RO6926496, RO7126209, RG6102, Brain shuttle gantenerumab, Rasagiline, Rasagiline mesylate, Azilect, TVP-1012, Rember™, Methylene Blue, methylthioninium (MT), TRx-0014, Repetitive Transcranial Magnetic Stimulation, rTMS, Resveratrol, trans-3,4′,5-trihydroxystilbene, Rilapladib, SB-659032, Riluzole, Rilutek®, RP 54274, Rivastigmine, Exelon™, Rivastigmine tartrate, Rivastach® Patch, Prometax®, SDZ ENA 713, Rofecoxib, Vioxx™, Rosiglitazone, Rosiglitazone maleate, Avandia, Rotigotine, Neupro, S 38093, S-Equol, Aus-131, S47445, CX1632, SAGE-718, SAR110894D, SAR228810, SB 202026, Memric, SDI-118, SGS-742, CGP-36742, DVD-742, ST101, ZSET1446, SUVN-G3031, Sabeluzole, R58735, Saracatinib, AZD0530, Sargramostim, GM-CSF Leukine, Leukine®, Semagacestat, LY450139 Dihydrate, hydroxylvaleryl monobenzocaprolactam, Semaglutide Ozempic, Rybelsus, Sembragiline, RO4602522, RG1577, Semorinemab, RO7105705, MTAU9937A, RG6100, Simufilam, PTI-125, sumifilam, Simvastatin, Zocor®, Lipex®, Lipovas®, Denan®, Solanezumab LY2062430, Souvenaid, Fortasyn Connect, Suritozole, MD 26479, Suvorexant, Belsomra, MK-4305, T3D-959, DB959, TB006, TPI 287, Tacrine, Cognex™, Telmisartan, Micardis, Thalidomide, Thalomid®, Tideglusib, NP031112, Nypta®, Zentylor™, Glycogen synthase kinase 3 inhibitor, NP12, Tilavonemab, ABBV-8E12, C2N 8E12, HJ8.5, Trazodone, Trazodone hydrochloride, Oleptro, Desyrel, Tricaprilin, AC-1204, Caprylic triglyceride, Troriluzole, BHV-4157, trigriluzole, FC-4157, UB-311, Umibecestat, CNP520, BACE Inhibitor, Vafidemstat, ORY-2001, Valacyclovir, Valtrex, Valaciclovir, 256U87, Valproate, Depakote, Depakene, Valproic acid, Divalproex sodium, Vanutide cridificar, ACC-001, PF-05236806, Varenicline, Champix™, Chantix™, Varenicline, tartrate, CP-526555, Alpha4 beta2 nicotinic receptor agonist, Varoglutamstat, PQ912, Vascepa, Icosapent ethyl (IPE), Ethyl eicosapentaenoic acid (E-EPA), AMR101, Miraxion, Verubecestat, MK-8931, MK-8931-009, BACE inhibitor, XProl595, Pegipanermin, INB03, NeuLiv, XENP1595, DN-TNF, XENP345, XPro™, LIVNate, Xaliproden, SR 57746A, Xanamem, UE 2343, Young Plasma, Zagotenemab, LY3303560, kMCT-ONS, medium chain triglyceride-based ketogenic oral nutritional supplement, kMCT, and/or a combination therein. See alzforum(dot)org/therapeutics/search?fda_statuses=&target_types=&therapy_types=&conditions%5B%5D=145&keywords-entry=&keywords=#results.

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as bags, vials, tubes, and the like, each of the container(s) comprising one of the separate elements to be used in a method described herein. Suitable containers include, for example, bags, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, tubes, bags, containers, bottles, and any packaging material suitable for a selected formulation and intended mode of administration and treatment.

For example, the container(s) include NK cells as disclosed herein and/or one or more secondary Alzheimer's disease therapies or therapeutics. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. In some embodiments, the label complies with one or more regulations for pharmaceutical and/or investigative use. For example, in some embodiments, the label comprises the statement “Caution: New Drug—Limited by Federal (or United States) law to investigational use.”

In some embodiments, the pharmaceutical compositions are presented in a pack or dispenser device which contains one or more unit dosage forms containing a compound provided herein. The pack, for example, contains metal or plastic foil, such as a blister pack. In some embodiments, the pack or dispenser device is accompanied by instructions for administration. In some embodiments, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are also prepared, placed in an appropriate container, and labeled for treatment of an indicated condition.

### Numbered Arrangements

Some embodiments provided herein are described by way of the following provided numbered arrangements and also provided as possible combinations or overlapping embodiments:
1. A method of treating Alzheimer's disease in a subject, the method comprising:
   a. identifying a subject, wherein the subject has Alzheimer's; and
   b. administering to the subject an expanded natural killer (NK) cell population, wherein the NK cells are expanded by a method comprising:
      i) isolating at least one of CD56+ cells and/or CD3−/CD56+ cells from the PBMCs;
      ii) co-culturing the at least one of CD56+ cells and/or CD3−/CD56+ cells with a combination of feeder cells in the presence of at least two cytokines;
      iii) wherein the combination of feeder cells comprises irradiated Jurkat cells and irradiated Epstein-Barr virus transformed lymphocyte continuous line (EBV-LCL) cells; and
      iv) wherein the at least two cytokines comprise IL-2 and IL-21.
2. The method of arrangement 1, wherein the amount of expanded NK cells administered to a subject is a therapeutically effective amount.
3. The method of arrangement 2, wherein the therapeutically effective amount of expanded NK cells comprises 0.1×10⁹ to 9×10⁹ cells.
4. The method of arrangement 1, wherein at least 1, 2, 3, 4, 5, 6, 7, 8, 9, 10, 11, 12, 13, 14, 15, 16, 17, 18, 19, 20 or more doses of expanded NK cells is administered to the subject.
5. The method of arrangement 1, wherein IL-2 is added at a concentration of 50-1000 IU/mL during step ii).
6. The method of arrangement 1, wherein IL-21 is added at a concentration of 10-100 ng/mL during step ii).
7. The method of arrangement 1, wherein the Mini-Mental State Exam (MMSE) score of the subject is between 24-30, 19-23, or 10-18 after treatment with expanded NK cells.
8. The method of arrangement 1, wherein the MMSE score of the subject is ≥24 after treatment with expanded NK cells.
9. The method of arrangement 1, wherein the MMSE score of the subject is ≥19 after treatment with expanded NK cells.
10. The method of arrangement 1, wherein the MMSE score of the subject is ≥10 after treatment with expanded NK cells.
11. The method of arrangement 1, further comprising:
   co-culturing the at least one of CD56+ cells and/or CD3−/CD56+ cells with the combination of feeder cells, in the presence of IL-2 for a first period; and
   co-culturing the at least one of CD56+ cells and/or CD3−/CD56+ cells with the combination of feeder cells, in the presence of IL-21 for a second period.
12. The method of arrangement 11, wherein IL-21 is added more than once during Day 0-6 of the second period.
13. The method of arrangement 11, wherein IL-21 and the combination of feeder cells are added more than once during Day 0-6 of the second period.
14. The method of arrangement 11, wherein IL-21 is added more than once during the first six days of every fourteen-day cycle during the second period.
15. A method of cell therapy comprising:
   a. identifying a subject, wherein the subject has Alzheimer's disease; and
   b. administering to the subject an expanded NK cell population, wherein the NK cells are expanded by a method comprising:
      i) isolating at least one of CD56+ cells and/or CD3−/CD56+ cells from the PBMCs;
      ii) co-culturing the at least one of CD56+ cells and/or CD3−/CD56+ cells with a combination of feeder cells in the presence of at least two cytokines;
      iii) wherein the combination of feeder cells comprises irradiated Jurkat cells and irradiated Epstein-Barr virus transformed lymphocyte continuous line (EBV-LCL) cells; and
      iv) wherein the at least two cytokines comprise IL-2 and IL-21.
16. The method of Arrangement 15, wherein the amount of expanded NK cells administered to a subject is a therapeutically effective amount.
17. The method of Arrangement 16, wherein the therapeutically effective amount of expanded NK cells comprises 0.1×10⁹ to 9×10⁹ cells.
18. The method of Arrangement 15, wherein at least 1, 2, 3, 4, 5, 6, 7, 8, 9, 10, 11, 12, 13, 14, 15, 16, 17, 18, 19, 20 or more doses of expanded NK cells is administered to the subject.
19. The method of Arrangement 15, wherein IL-2 is added at a concentration of 50-1000 IU/mL during step ii).
20. The method of Arrangement 15, wherein IL-21 is added at a concentration of 10-100 ng/mL during step ii).
21. The method of Arrangement 15, wherein the Mini-Mental State Exam (MMSE) score of the subject is between 24-30, 19-23, or 10-18 after treatment with expanded NK cells.
22. The method of arrangement 15, wherein the MMSE score of the subject is ≥24 after treatment with expanded NK cells.
23. The method of arrangement 15, wherein the MMSE score of the subject is ≥19 after treatment with expanded NK cells.
24. The method of arrangement 15, wherein the MMSE score of the subject is ≥10 after treatment with expanded NK cells.
25. The method of arrangement 15, further comprising:
   co-culturing the at least one of CD56+ cells and/or CD3−/CD56+ cells with the combination of feeder cells, in the presence of IL-2 for a first period; and
   co-culturing the at least one of CD56+ cells and/or CD3−/CD56+ cells with the combination of feeder cells, in the presence of IL-21 for a second period.
26. The method of arrangement 25, wherein IL-21 is added more than once during Day 0-6 of the second period.
27. The method of arrangement 25, wherein IL-21 and the combination of feeder cells are added more than once during Day 0-6 of the second period.
28. The method of arrangement 25, wherein IL-21 is added more than once during the first six days of every fourteen-day cycle during the second period.
29. A population of expanded NK cells, wherein the NK cells were expanded by a method that comprises:
   i) isolating at least one of CD56+ cells and/or CD3−/CD56+ cells from the PBMCs;
   ii) co-culturing the at least one of CD56+ cells and/or CD3−/CD56+ cells with a combination of feeder cells in the presence of at least two cytokines;
   iii) wherein the combination of feeder cells comprises irradiated Jurkat cells and irradiated Epstein-Barr virus transformed lymphocyte continuous line (EBV-LCL) cells; and
   iv) wherein the at least two cytokines comprise IL-2 and IL-21; and
   wherein the population of expanded NK cells has been administered to a subject who has Alzheimer's disease.
30. The population of cells of arrangement 29, wherein the amount of expanded NK cells administered to a subject is a therapeutically effective amount.
31. The method of arrangement 30, wherein the therapeutically effective amount of expanded NK cells comprises 0.1×10⁹ to 9×10⁹ cells.
32. The population of cells of arrangement 29, wherein at least 1, 2, 3, 4, 5, 6, 7, 8, 9, 10, 11, 12, 13, 14, 15, 16, 17, 18, 19, 20 or more doses of expanded NK cells is administered to the subject.
33. The population of cells of Arrangement 29, wherein IL-2 is added at a concentration of 50-1000 IU/mL during step ii).
34. The population of cells of Arrangement 29, wherein IL-21 is added at a concentration of 10-100 ng/mL during step ii).
35. The population of cells of Arrangement 29, wherein the Mini-Mental State Exam (MMSE) score of the subject is between 24-30, 19-23, or 10-18 after treatment with expanded NK cells.
36. The population of cells of Arrangement 29, wherein the MMSE score of the subject is ≥24 after treatment with expanded NK cells.
37. The population of cells of Arrangement 29, wherein the MMSE score of the subject is ≥19 after treatment with expanded NK cells.
38. The population of cells of Arrangement 29, wherein the MMSE score of the subject is 10 after treatment with expanded NK cells.
39. The population of cells of arrangement 29, further comprising:
   co-culturing the at least one of CD56+ cells and/or CD3−/CD56+ cells with the combination of feeder cells, in the presence of IL-2 for a first period; and
   co-culturing the at least one of CD56+ cells and/or CD3−/CD56+ cells with the combination of feeder cells, in the presence of IL-21 for a second period.
40. The population of cells of arrangement 39, wherein IL-21 is added more than once during Day 0-6 of the second period.
41. The population of cells of arrangement 39, wherein IL-21 and the combination of feeder cells are added more than once during Day 0-6 of the second period.
42. The population of cells of arrangement 39, wherein IL-21 is added more than once during the first six days of every fourteen-day cycle during the second period.
43. A method of treating Alzheimer's disease in a subject, the method comprising:
   a. identifying a subject, wherein the subject has Alzheimer's disease; and
   b. administering to the subject a therapeutically effective amount of a NK cell population (e.g., an autologous NK cell population).
44. The method of any one of arrangements 1-43 further comprising administration of one or more secondary Alzheimer's disease therapeutics.
45. The method of arrangement 44, wherein the one or more secondary Alzheimer's disease therapeutics comprises aducanumab.
46. The method of arrangement 44, wherein the NK cells and the one or more secondary Alzheimer's disease therapeutics are co-administered.
47. The method of arrangement 44, wherein the NK cells and the one or more secondary Alzheimer's disease therapeutics are administered for 1, 2, 3, 4, 5, 6, 7, 8, 9, 10, 11, 12, 14, 16, 20, 24, 28, 32, or 36 weeks.
48. The method of arrangement 44, wherein the NK cells and the one or more secondary Alzheimer's disease therapeutics are co-administered for 1, 2, 3, 4, 5, 6, 7, 8, 9, 10, 11, 12, 14, 16, 20, 24, 28, 32, or 36 weeks.
49. The method of arrangement 44, wherein the NK cells and the one or more secondary Alzheimer's disease therapeutics are alternately administered for 1, 2, 3, 4, 5, 6, 7, 8, 9, 10, 11, 12, 14, 16, 20, 24, 28, 32, or 36 weeks.
50. The method of arrangement 44, wherein administration of the NK cells in combination with one or more secondary Alzheimer's therapies and/or therapeutics, reduces the time required for the NK cells to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
51. The method of arrangement 44, wherein administration of the NK cells in combination with one or more secondary Alzheimer's therapies and/or therapeutics, reduces the time required for the one or more secondary Alzheimer's disease therapeutics to achieve a therapeutic effect by 1-fold, 2-fold, 3-fold, 4-fold, 5-fold, 6-fold, 7-fold, 8-fold, 9-fold, or 10-fold
52. The method of arrangement 44, wherein administration of the NK cells in combination with one or more secondary Alzheimer's therapies and/or therapeutics, reduces the time required for the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
53. The method of arrangement 44, wherein administration of the NK cells in combination with one or more secondary Alzheimer's therapies and/or therapeutics, reduces the time required for the NK cells to achieve a therapeutic effect by 1-fold, 2-fold, 3-fold, 4-fold, 5-fold, 6-fold, 7-fold, 8-fold, 9-fold, or 10-fold
54. The method of arrangement 44, wherein administration of the NK cells in combination with one or more secondary Alzheimer's therapies and/or therapeutics, reduces the number of doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
55. The method of arrangement 44, wherein administration of the NK cells in combination with one or more secondary Alzheimer's therapies and/or therapeutics, reduces the number of doses of the NK cells required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
56. The method of arrangement 44, wherein administration of the NK cells in combination with one or more secondary Alzheimer's therapies and/or therapeutics, reduces the doses of the one or more secondary Alzheimer's disease therapeutics required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
57. The method of arrangement 44, wherein administration of the NK cells in combination with one or more secondary Alzheimer's therapies and/or therapeutics, reduces the doses of the NK cells required to achieve a therapeutic effect by 5%, 10%, 15%, 20%, 25%, 30%, 40%, 50%, 60%, 70%, 75%, 80%, 90%, 91%, 92%, 93%, 94%, 95%, 96%, 97%, 98%, or 99%.
58. The method of arrangement 44, wherein administration of the NK cells in combination with one or more secondary Alzheimer's therapies and/or therapeutics, reduces the number of doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.
59. The method of arrangement 44, wherein administration of the NK cells in combination with one or more secondary Alzheimer's therapies and/or therapeutics, reduces the number of doses of the NK cells required to achieve a therapeutic effect by 1-fold, 2-fold, 3-fold, 4-fold, 5-fold, 6-fold, 7-fold, 8-fold, 9-fold, or 10-fold.
60. The method of arrangement 44, wherein administration of the NK cells in combination with one or more secondary Alzheimer's therapies and/or therapeutics, reduces the doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.
61. The method of arrangement 44, wherein administration of the NK cells in combination with one or more secondary Alzheimer's therapies and/or therapeutics, reduces the doses of the NK cells required to achieve a therapeutic effect by 1-fold, 2-fold, 3-fold, 4-fold, 5-fold, 6-fold, 7-fold, 8-fold, 9-fold, or 10-fold.
62. A kit comprising the NK cell population of any of the preceding arrangements and one or more secondary Alzheimer's disease therapeutics.
63. A formulation comprising the NK cell population of any of the preceding arrangements and one or more secondary Alzheimer's disease therapeutics.
64. A composition comprising the NK cell population of any of the preceding arrangements and one or more secondary Alzheimer's disease therapeutics.
65. The method of any one of the preceding arrangements, wherein identifying a subject as having Alzheimer's disease comprises detecting and/or quantifying one or more biomarkers.
66. The method of arrangement 65, wherein the one or more biomarkers are quantified and/or detected in the subject's cerebrospinal fluid and/or plasma from peripheral blood.
67. The method of arrangement 65, wherein the one or more biomarkers comprise YKL-40, CX3CL1, TNF-α, IL-6, IL-8, IL-12/IL-23p40, and/or sTREM2, or any combination thereof.
68. The method of arrangement 65, wherein the one or more biomarkers comprise Aβ-42/40, Aβ-42, total tau, p-tau, GFAP, and/or NfL, or any combination thereof.
69. The method of any one of the preceding arrangements, wherein identifying a subject as having Alzheimer's disease comprises administering one or more cognitive assessments.
70. The method of arrangement 69, wherein the one or more cognitive assessments comprises a Clinical Dementia Rating, Alzheimer's disease assessment scale-cognitive subscale, mini-mental status exam, or any combination thereof.
71. The method of any one of the preceding arrangements, wherein administration of the NK cells increases the Aβ-42 level in the subject's CSF and/or plasma by about 28-275%.
72. The method of any one of the preceding arrangements, wherein administration of the NK cells increases the Aβ-42/40 ratio in the subject's CSF and/or plasma by about 40-264%.
73. The method of any one of the preceding arrangements, wherein administration of the NK cells increases the IL-8 level in the subject's CSF and/or plasma by about 25-180%.
74. The method of any one of the preceding arrangements, wherein administration of the NK cells decreases the p-tau level in the subject's CSF and/or plasma by about 21-84%.
75. The method of any one of the preceding arrangements, wherein administration of the NK cells decreases the GFAP level in the subject's CSF and/or plasma by about 36-95%.
76. The method of any one of the preceding arrangements, wherein administration of the NK cells decreases NfL level in the subject's CSF and/or plasma by about 4-71%.
77. The method of any one of the preceding arrangements, wherein administration of the NK cells increases the CX3CL1 level in the subject's CSF and/or plasma by about 18-231%.
78. The method of any one of the preceding arrangements, wherein administration of the NK cells decreases the IL-6 level in the subject's CSF and/or plasma by about 19-65%.
79. The method of any one of the preceding arrangements, wherein administration of the NK cells decreases the TNF-α level in the subject's CSF and/or plasma by about 42-96%.
80. The method of any one of the preceding arrangements, wherein administration of the NK cells decreases the IL-12/IL-23p40 ratio in the subject's CSF and/or plasma by about 7-53%.
81. The method of any one of the preceding arrangements, wherein administration of the NK cells decreases neuroinflammation in the subject as compared to the level of neuroinflammation in the subject prior to administration of the NK cells.
82. The method of any one of the preceding arrangements, wherein administration of the NK cells decreases neuroinflammation in the subject by up to about 100% as compared to the level of neuroinflammation in the subject prior to administration of the NK cells.
83. The method of any one of the preceding arrangements, wherein the NK cells are administered intravenously.
84. The method of any one of the preceding arrangements, wherein the NK cells are intravenously administered weekly for up to 20 weeks.
85. The method of any one of the preceding arrangements, wherein the expanded NK cell population or the NK cell population is or comprises SNK01.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

### EXAMPLES

The following examples are provided to illustrate certain particular features and/or embodiments. These examples should not be construed to limit the disclosure to the particular features or embodiments described.

### Example 1. Isolation of CD56+ Natural Killer (NK) Cells

CD56+ cells and CD3−/CD56+ cells will be isolated from PBMCs by the following method. First, the PBMCs will be isolated from the blood using a Ficoll-Hypaque density gradient method and then the cells will be counted.

### Example 1-1. Isolation of CD56+ Cells

The counted PBMCs will be added with a MACS buffer (1×PBS+0.5% HSA) and suspended, and added with CD56 microbeads (Miltenyi Biotec) to be 1 to 20 μL per 1.0×10⁷ PBMCs, and then incubated at 2 to 8° C. for 5 to 30 minutes. After incubation, the MACS buffer will be added and mixed, and then the mixture will be centrifuged (600×g) to precipitate the cells. After centrifugation, a supernatant will be removed, and the cells will be suspended by adding the MACS buffer and added in a column connected to a MACS separator. The MACS buffer will be passed through the column to remove non-specific binding. The column will be separated from the MACS separator and transferred to a 15 mL conical tube, and then added with the MACS buffer to isolate CD56+ cells attached to the column.

### Example 1-2. Isolation of CD3−/CD56+ Cells

The counted PBMCs will be added with a MACS buffer (1×PBS±0.5% HSA) and suspended, and added with CD3 microbeads (Miltenyi Biotec) to be 1 to 20 μL per 1.0×10⁷ PBMCs, and then incubated at 2 to 8° C. for 5 to 30 minutes. After incubation, the MACS buffer will be added and mixed, and then the mixture will be centrifuged (600×g) to precipitate the cells. After centrifugation, a supernatant will be removed, and the cells will be suspended by adding the MACS buffer and added in a column connected to a MACS separator. The MACS buffer passed through the column to collect CD3− cells. The collected CD3− cells will be added with a MACS buffer (1×PBS+0.5% HSA) and suspended and added with CD56 microbeads (Miltenyi Biotec) to be 1 to 20 μL per 1.0×10⁷ CD3− cells, and then incubated at 2 to 8° C. for 5 to 30 minutes. After incubation, the MACS buffer will be added and mixed, and then the mixture will be centrifuged (600×g) to precipitate the cells. After centrifugation, a supernatant will be removed, and the cells will be suspended by adding the MACS buffer and added in a column connected to a MACS separator. The MACS buffer will be passed through the column to remove non-specific binding. The column will be separated from the MACS separator and transferred to a 15 mL conical tube, and then added with the MACS buffer to isolate CD3−/CD56+ cells attached to the column.

### Example 1-3. Production of NK Cells Using the CD56+ Cells and CD3−/CD56+ Cells

The CD56+ cells or the CD3−/CD56+ cells isolated from the PBMCs as in Examples 1-1 and 1-2 will be added in a RPMI-1640 medium containing FBS 10% added with IL-2 at a concentration of 500 IU/mL together with prepared combination of feeder cells (Jurkat cells and EBV-LCL cells) irradiated with 100 Gy radiation and then co-cultured in an incubator at 37° C. and 5% CO₂. The ratio of (CD56+ cells and/or CD3−/CD56+ cells):(Jurkat cells):(EBV-LCL cells) will be about 1:30:30.

Meanwhile, the Jurkat cells may be obtained from ATCC (ATCC TIB-152), and the EBV-LCL cells will be prepared by the following method: 30×10⁶ PBMCs will be added in 9 mL of a culture medium, the mixture will be added in a T 25 culture flask, and then 9 m of an EBV supernatant will be added. 80 μL of cyclosporine A will be added and then cultured at 37° C. After 7 days of culture, a half of supernatant will be removed, a fresh culture medium will be added, and then 40 μL of cyclosporine A will be added. The same process as the 7th day will be repeated once every 7 days until 28 days of culture. The cell line will be usable after 28 days of culture, and from this time, the cell line will be cultured in the culture medium without adding cyclosporine A.

### Example 2. Production of CD56+ Natural Killer (NK) Cells (IL-2/IL-21 Treated)

NK cells will be produced using same method of Example 1 (1-1 to 1-3), except for adding IL-2 (500 IU/mL) and IL-21 (50 ng/mL) instead of IL-2 (500 IU/mL).

### Comparative Example 1. Production of Natural Killer (NK) Cells without the CD56+ Cells Isolation Step (IL-2 Treated)

PBMCs will be isolated from the blood using a Ficoll-Hypaque density gradient method. The PBMCs will be added in a RPMI-1640 medium containing FBS 10% added with IL-2 at a concentration of 500 IU/mL together with prepared feeder cells (Jurkat cells and EBV-LCL cells) irradiated with 100 Gy irradiation and then co-cultured in an incubator at 37° C. and 5% CO₂.

### Comparative Example 2. Production of Natural Killer (NK) Cells without the CD56+ Cells Isolation Step (IL-2/IL-21 Treated)

NK cells will be produced using same method of Comparative Example 1, except for adding IL-2 (500 IU/mL) and IL-21 (50 ng/mL) instead of IL-2 (500 IU/mL).

### Comparative Examples 3&4. Production of Natural Killer (NK) Cells without the CD56+ Cells Isolation Step

NK cells will be produced using similar methods of Comparative Examples 1&2, respectively, except for that a ratio of PBMC:(Jurkat cells):(EBV-LCL cells) will be 1:0.5:0.5.

### Example 5. Production of CD56+ Cells and CD3/CD56+ NK Cells with Freeze-Thawing

This non-limiting example shows IL-21 enhancing expansion of CD56+ and CD3−/CD56+ NK cells with freeze-thawing.

### Example 5-1. Preparation of CD56+ Natural Killer Cells (NK Cells)−1

First, blood PBMC will be isolated using a Ficoll density gradient (Ficoll-Hypaque density gradient method). The PBMC will further be treated according to 5-2 or 5-3 below.

### Example 5-2. CD56+ Cell Isolation

The PBMC will be suspended by addition of MACS buffer (1×PBS+0.5% HSA) and CD56 microbeads (Miltenyi Biotec) will be added to obtain 1-20 μL per 1.0×10⁷ PBMC and incubated for 5-30 minutes at 2-8° C. After incubation, MACS buffer will be added and mixed, and the mixture will be centrifuged (600×g) to precipitate the cells. After centrifugation, the supernatant will be removed and MACS buffer will be added to resuspend the cells, and the cells will be added to a MACS separator coupled to a column. MACS buffer will be passed through the column to remove non-specific binding. The column will be separated from the MACS separator and transferred to a 15 mL conical tube, and MACS buffer will be added to isolate CD56+ cells attached to the column.

### Example 5-3. CD3−/CD56+ Cell Isolation

CD3−/CD56+ cells will be isolated as follows. The PBMC will be suspended by addition of MACS buffer (1×PBS+0.5% HSA) and CD3 microbeads (Miltenyi Biotec) will be added to obtain 1-20 μL per 1.0×10⁷ PBMC and incubated for 5-30 minutes at 2-8° C. After incubation, MACS buffer will be added and mixed, and the mixture will be centrifuged (600×g) to precipitate the cells. After centrifugation, the supernatant will be removed and MACS buffer will be added to resuspend the cells, and the cells will be added to a MACS separator coupled to a column. MACS buffer will be passed through the column to recover CD3− cells.

The MACS buffer (1×PBS+0.5% HSA) will be added to the recovered CD3− cells to resuspend the CD3− cells and CD56 microbeads (Miltenyi Biotec) will be added to obtain 1-20 μL per 1.0×10⁷ CD3− cells and incubated at 2 to 8° C. for 5 to 30 minutes. After incubation, MACS buffer will be added and mixed, and the mixture was centrifuged (600×g) to precipitate the cells. After centrifugation, the supernatant will be removed and MACS buffer will be added to resuspend the cells, and the cells will be added to a MACS separator coupled to a column. MACS buffer will be passed through the column to remove non-specific binding. The column will be separated from the MACS separator and transferred to a 15 mL conical tube, and MACS buffer will be added to isolate CD3−/CD56+ cells attached to the column.

### Example 5-4. Primary Culture

The separated CD56+ cells or CD3−/CD56+ cells from 5-2 and 5-3 will each be co-cultured in an incubator with feeder cells (Jurkat cells, and EBV-LCL cells) previously prepared by 100 Gy irradiation with and in the presence of IL-2 and IL-21 at 500 IU/mL and 50 ng/mL concentration, respectively, in RPMI-1640 medium with 10% FBS at 37° C., 5% CO2.

On day 6, cells will be inoculated at 1.0×10⁵-2.0×10⁶/mL in a 350 mL standard bag and cultivated for four additional days, and on day 10 the cells will be inoculated at 1.0×10⁵-2.0×10⁶ cells/mL, in a 1 L bag and cultured for another 4 days. At this time, the ratio (CD56+ cells or CD3−/CD56+ cells):(Jurkat cell):(EBV-LCL cell) is 1:30:30 during incubation.

### Example 5-5. Secondary Culture after Freezing and Thawing

On the fourteenth day of culture as mentioned in Example 5-4, the cultured cells will be suspended in a solution containing 90% FBS and 10% DMSO, stored frozen at −192° C. or lower, and thawed in a 37° C. constant temperature water bath according to the culture schedule.

Then, RPMI-1640 containing 10% of FBS to which IL-2 and IL-21 will be added at a concentration of 500 IU/mL and 50 ng/mL, respectively, along with 100 Gy irradiated feeder cells (Jurkat cells and EBV-LCL cells). After putting in the medium, it will be co-cultured in a 37° C., 5% CO2 incubator.

On day 6 after thawing and culturing, the cells will be inoculated into a 350 mL bag (at 1.0×10⁵-2.0×10⁶ cells/mL and incubated for an additional four days, and on day 10 the cells will be inoculated into a 1 L standard bag at 1.0×10⁵-2.0×10⁶ cells/mL and further cultured for 4 days.

In order to sustain the growth of cells in culture until 17-18 days after thawing, cells will be co-cultured with 100 Gy irradiated feeder cells (Jurkat cells and EBV-LCL cells) in the presence of IL-2 and IL-21 at a concentration of 500 IU/mL and 50 ng/mL, respectively. Cells will be cultured in RPMI-1640 medium containing 10% FBS, in an incubator at 37° C., 5% CO2.

On the 6th day after thawing, the cells will be inoculated into a 1 L bag at 1.0×10⁵-2.0×10⁶ cells/mL, followed by additional culture for 4 days, and on the 10th day of culture after thawing, the cells will be inoculated into a IL bag at 1.0×10⁵-2.0×10⁶ cells/mL and further cultured for 4 days.

Finally, on the 28th day of culture after thawing, the cells will be inoculated into a 1 L bag at 1.0×10⁵-2.0×10⁶ cells/mL, followed by additional culture for an additional 3-4 days.

### Example 5-6. Preparation of CD56+ Natural Killer Cells−2

Natural killer cells will be prepared in the same manner as in 5-1, except for the step of adding cytokines in 5-5.

### Comparative Example 6. Preparation of Natural Killer Cells Excluding Cytokine Treatment Steps

Natural killer cells will be prepared in the same manner as in 5-1, except for the step of adding cytokine (IL-21) in 5-4 and 5-5.

### Example 5-7. Confirmation of NK Cell Proliferation Ability

The proliferative ability of NK cells cultured by the methods of 5-1 to 5-6 were measured. When the cytokine was not treated during the primary culture (IL-21−/−); (see comparative example 6 above), it was found that a sufficient number of NK cells for clinical application was not produced after the freezing and thawing process (data not shown). On the other hand, when the cells were treated with cytokine (IL-21+/+; see (5-1) above), NK cells were produced in sufficient numbers for clinical application even after the freezing and thawing process, and these results were not only when the cytokine was treated after the freezing and thawing process. In the case where cells were not treated with cytokine after freezing and thawing (IL-21+/−; see (5-6) above), the NK cells expanded as well as those treated with cytokine after freezing and thawing (data not shown).

### Experimental Example 7. Treatment of Alzheimer's Disease Patients with NK Cells

CD56+ NK cells will be produced according to the method of Examples 1, 2 and Comparative Examples 1, 2 for 17-18 days, except that PBMCs of Alzheimer's disease patients will be used. With respect to each of the NK cells cultured in a CO₂ incubator according to Examples 1, 2 and Comparative Examples 1, 2, on Day 6 of culture in a T 25 culture flask, cells will be inoculated into a 350 mL bag at 1.0×10⁵ to 2.0×10⁶/mL and further cultured for 4 days. On Day 10 of culture, the cells will be inoculated into a 1 L bag at 1.0×10⁵ to 2.0×10⁶/mL and then further cultured for 4 days. Finally, on Day 14 of culture, the cells will be inoculated into a 1 L bag at 1.0×10⁵ to 2.0×10⁶/mL and then further cultured for 3 to 4 days.

The subjects with Alzheimer's disease will be injected up to 20 times with NK cells at weekly intervals intravenously. The therapeutically effective amount of expanded NK cells comprises up to 9×10⁹ cells, including ranges in between. NK cells will be added repeatedly until improvement in Alzheimer's symptoms is achieved.

Cognitive and motor functions of the patient will be monitored at 1, 3, 6, 12 months. After 4-12 months, the NK cell-treated group will exhibit improved cognitive and motor functions.

### Example 7-1. Treatment of Alzheimer's Disease Patients with NK Cells and Aducanumab

CD56+ NK cells will be produced according to the method of Examples 1, 2 and Comparative Examples 1, 2 for 18 days, except that PBMCs of Alzheimer's disease patients will be used. With respect to each of the NK cells cultured in a CO₂ incubator according to Examples 1, 2 and Comparative Examples 1, 2, on Day 6 of culture in a T 25 culture flask, cells will be inoculated into a 350 mL bag at 1.0×10⁵ to 2.0×10⁶/mL and further cultured for 4 days. On Day 10 of culture, the cells will be inoculated into a 1 L bag at 1.0×10⁵ to 2.0×10⁶/mL and then further cultured for 4 days. Finally, on Day 14 of culture, the cells will be inoculated into a 1 L bag at 1.0×10⁵ to 2.0×10⁶/mL and then further cultured for 3 to 4 days.

The subjects with Alzheimer's disease will be injected up to 20 times with NK cells at weekly intervals intravenously. The therapeutically effective amount of expanded NK cells comprises 0.1×10⁹ to 1×10¹² cells, including ranges in between.

The subjects with Alzheimer's disease will also be injected up to 14 times with aducanumab at 4 week intervals intravenously. The therapeutically effective amount of aducanumab comprises 1 mg/kg to 10 mg/kg, including ranges in between. The subject dosage of aducanumab will be titrated, beginning at 1 mg/kg and increasing to 10 mg/kg from the seventh dose onward.

NK cells and aducanumab will be added repeatedly until improvement in Alzheimer's symptoms is achieved.

Cognitive and motor functions of the patient will be monitored at 1, 3, 6, 12 months. After 4-12 months, the NK cell-aducanumab treated group will exhibit improved cognitive and motor functions.

### Example 8. Treatment of Advanced Alzheimer's Patients with NK Cells

Three different patients with advanced Alzheimer's disease were treated with NK cells and improvement in their cognitive and motor functions was monitored over time. MMSE score ranking can be used to identify patients with advanced Alzheimer's.

### Example 8-1. Treatment of a Male Advanced Alzheimer's Patient with NK Cells

The subject was a 36-year-old male with advanced Alzheimer's. The subject weighed 170 pounds and the subject's blood sugar was 111 mg/dl. The subject's dementia panel of the molecular genetics report classified him as positive in PSEN1. The subject was classified under ICD-10-CM code as G31.84 with mild cognitive impairment which is stated as G93.0 lesion of brain. The subject was heterozygous in the PSEN1 gene for a variant designated c.338T>A, which is predicted to result in the amino acid substitution p.Leu113G1n. The variant c.338T>A has been previously reported in a patient with Alzheimer's disease (Finckh et al, 2005). A different amino acid change in the same position has been shown to be causative for frontotemporal dementia and early-onset Alzheimer's disease (Raux et al, 2000). The dementia panel of molecular genetics report of the subject is shown in Table 1.

TABLE 1

| | | DNA | | Highest | | |
|---|---|---|---|---|---|---|
| | Mode of | Variation, | | Allele | | |
| | inheritance, | Predicted | | frequency | In Silico | |
| Gene | Gene | Effects, | | in a gnomAD | Missense | |
| Transcript | OMIM | zygosity | ClinVar ID | Population | Predictions | Interpretation |
| PSEN1, | AD, 104311 | c.338T > A, | Not listed | Not | Damaging | Likely |
| NM_000021.3 | | p, Leu113Cln, | In ClinVar | present | | Pathogenic |
| | | Heterozygous | | | | |

The subject was injected with 7 doses of 2×10⁹ to 8×10⁹ NK cells as shown in Table 2. A brain PET scan was performed 30 minutes after injection of NK cells. Axial, coronal and sagittal tomographic views of the brain were evaluated, and a non-contrast CT was performed for attenuation correction and registration. After the injection of NK cells, the subject exhibited a striking decrease in parietal activity with moderate diffuse temporal, occipital and posterior fossa activity. The motor skills of the patient improved after injecting 7 doses of NK cells.

TABLE 2

| | | |
|---|---|---|
| Treatment #1 | Date | Jul. 13, 2020 |
| | Dosage | 4 × 10⁹ |
| Treatment #2 | Date | Aug. 3, 2020 |
| | Dosage | 4 × 10⁹ |
| Treatment #3 | Date | Aug. 24, 2020 |
| | Dosage | 2 × 10⁹ |
| Treatment #4 | Date | Sep. 21, 2020 |
| | Dosage | 4 × 10⁹ |
| Treatment #5 | Date | Oct. 26, 2020 |
| | Dosage | 8 × 10⁹ |
| Treatment #6 | Date | Nov. 23, 2020 |
| | Dosage | 6 × 10⁹ |
| Treatment #7 | Date | Mar. 23, 2021 |
| | Dosage | 4 × 10⁹ |

### Example 8-2. Treatment of a Female Advanced Alzheimer's Patient with NK Cells

The subject was a 72-year-old female with advanced Alzheimer's disease. The subject was treated with 19 doses of 0.4×10⁹ to 8×10⁹ NK cells as shown in Table 3. The cognitive and motor skills of the subject improved following 5 treatments with NK cells as shown in Table 4A-C below.

TABLE 3

| | | |
|---|---|---|
| Treatment #1 | Date | Sep. 14, 2020 |
| | Dosage | 4 × 10⁹ |
| Treatment #2 | Date | Sep. 28, 2020 |
| | Dosage | 6 × 10⁹ |
| Treatment #3 | Date | Oct. 12, 2020 |
| | Dosage | 8 × 10⁹ |
| Treatment #4 | Date | Oct. 26, 2020 |
| | Dosage | 6 × 10⁹ |
| Treatment #5 | Date | Nov. 9, 2020 |
| | Dosage | 2 × 10⁹ |
| Treatment #6 | Date | Nov. 23, 2020 |
| | Dosage | 2 × 10⁹ |
| Treatment #7 | Date | Dec. 21, 2020 |
| | Dosage | 6 × 10⁹ |
| Treatment #8 | Date | Jan. 4, 2021 |
| | Dosage | 8 × 10⁹ |
| Treatment #9 | Date | Jan. 25, 2021 |
| | Dosage | 8 × 10⁹ |
| Treatment #10 | Date | Feb. 8, 2021 |
| | Dosage | 8 × 10⁹ |
| Treatment #11 | Date | Feb. 22, 2021 |
| | Dosage | 0.4 × 10⁹ |
| Treatment #12 | Date | Mar. 8, 2021 |
| | Dosage | 8 × 10⁹ |
| Treatment #13 | Date | Mar. 22, 2021 |
| | Dosage | 8 × 10⁹ |
| Treatment #14 | Date | Apr. 5, 2021 |
| | Dosage | 8 × 10⁹ |
| Treatment #15 | Date | Apr. 19, 2021 |
| | Dosage | 8 × 10⁹ |
| Treatment #16 | Date | May 3, 2021 |
| | Dosage | 4 × 10⁹ |
| Treatment #17 | Date | May 18, 2021 |
| | Dosage | 8 × 10⁹ |
| Treatment #18 | Date | May 31, 2021 |
| | Dosage | 8 × 10⁹ |
| Treatment #19 | Date | Jun. 14, 2021 |
| | Dosage | 8 × 10⁹ |

TABLE 4A

| | |
|---|---|
| Before Treatment | After 5 treatments |

MEMORY

| | |
|---|---|
| Had difficulty in naming members of | Got kids/no Grandkids |
| immediate family, children/grandchildren | |
| Could not name her siblings, brothers | Named 3 or 4 |
| and sisters | |
| Could not name her father or mother's | Named both |
| first name | |
| Could not remember Doctor's name | Got it |
| Could not name the town she lives in | Said NY state |
| Could not remember the name of her Aide | Got it |
| Could not name the Town she grew up in | Still no |
| Number sequence memory was limited to 3. | 4 consistently |
| Did not know Left from Right . . . arm, foot | Knew left/right |
| Could touch her nose not her Chin, Ear, Eye | Yes to all |
| Did not know or did not see Letter or Numbers | Recognized the letter A |
| Had no concept of time of day. Wanted to | Knew it was afternoon |
| go to bed at 9am for example | |
| Could not navigate rooms in her own home. | Knew kitchen |

SPACIAL/SIGHT

| | |
|---|---|
| Could not feed herself, use of fork or | Consistently used fork |
| spoon. Except in rare cases. | |
| Could not find things in a room . . . TV, | Still no |
| Toilet, Chair | |
| Could not draw or copy a simple | Still no |
| drawing . . . square/circle | |

LANGUAGE

| | |
|---|---|
| Had difficulty finding words or | Could talk in sentences, |
| putting sentences together | not a lot but sometimes |

### Example 8-3. Treatment of a Female Advanced Alzheimer's Patient with NK Cells

The subject was a 79-year-old female with advanced Alzheimer's. The subject was classified as having severe dementia based on her Mini-Mental State Exam (MMSE) score of 12 at the beginning of the treatment. The classifications of different stages of Alzheimer's disease based on the MMSE score is listed in Table 5.

TABLE 5

| MMSE Score | Stage |
|---|---|
| ≤12 | Severe dementia |
| 13-20 | Moderate dementia |
| 20-25 | Mild dementia |
| ≥25 | No cognitive impairment |

After treatment with NK cells, the subject's MMSE score improved over time as shown in Table 6.

TABLE 6

| Date | MMSE Score |
|---|---|
| Day 0 | 12 |
| Day 42 | 15 |
| Day 63 | 16 |
| Day 84 | 18 |
| Day 112 | 22 |
| Day 133 | 23 |
| Day 153 | 22 |
| Day 184 | 19 |
| Day 224 | 22 |

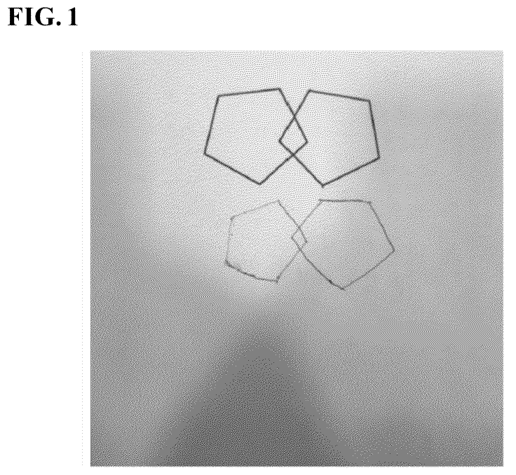

The subject's special or sight skills were tested by her ability to copy a simple diagram by following the instructions. The subject's special or sight skills improved following the treatment with NK cells as shown in FIG. 1.

### Example 9. Treatment of Advanced Alzheimer's Patients with NK Cells and Aducanumab

Patients with advanced Alzheimer's disease will be treated with NK cells and aducanumab. Improvement in their cognitive and motor functions will be monitored over time. MMSE score ranking can be used to identify patients with advanced Alzheimer's.

CD56+ NK cells will be produced according to the method of Examples 1, 2 and Comparative Examples 1, 2 for 17-18 days, except that PBMCs of Alzheimer's disease patients will be used. With respect to each of the NK cells cultured in a CO₂ incubator according to Examples 1, 2 and Comparative Examples 1, 2, on Day 6 of culture in a T 25 culture flask, cells will be inoculated into a 350 mL bag at 1.0×10⁵ to 2.0×10⁶/mL and further cultured for 4 days. On Day 10 of culture, the cells will be inoculated into a 1 L bag at 1.0×10⁵ to 2.0×10⁶/mL and then further cultured for 4 days. Finally, on Day 14 of culture, the cells will be inoculated into a 1 L bag at 1.0×10⁵ to 2.0×10⁶/mL and then further cultured for 3 to 4 days.

The subjects with advanced Alzheimer's disease will be injected up to 20 times with NK cells at weekly intervals intravenously. The therapeutically effective amount of expanded NK cells comprises 0.1×10⁹ to 1×10¹² cells, including ranges in between.

The subjects with advanced Alzheimer's disease will also be injected up to 14 times with aducanumab at 4 week intervals intravenously. The therapeutically effective amount of aducanumab comprises 1 mg/kg to 10 mg/kg, including ranges in between. The subject dosage of aducanumab will be titrated, beginning at 1 mg/kg and increasing to 10 mg/kg from the seventh dose onward.

NK cells and aducanumab will be added repeatedly until improvement in Alzheimer's symptoms is achieved.

Cognitive and motor functions of the patient will be monitored at 1, 3, 6, 12 months. After 4-12 months, the NK cell-aducanumab treated group will exhibit improved cognitive and motor functions.

### Example 10. Single Center. Open-Label, Phase 1 Study to Evaluate the Safety, Tolerability, and Exploratory Efficacy of SNK01 in Subjects with Mild Cognitive Impairment (MCI) and Alzheimer's Disease (AD) (Study SNK01-MX04)

Alzheimer's disease is a dual proteinopathy characterized by extracellular deposits of fibrillar amyloid-beta peptides and aggregates of the phosphorylated microtubule-associated protein tau in neurofibrillary tangles.

CSF specimens collected from subjects participating in a single center, open-label, phase 1 study to evaluate the safety, tolerability, and preliminary efficacy of SNK01 (autologous natural killer cell), as a single agent, in subjects with Alzheimer's disease, were used to Examine the level and change of AD biomarkers and cytokine/chemokine proteins by treatment of 3 different doses of SNK01 (www(dot)sec(dot)gov/ix?doc=/Archines/edgar/data/1845459/000110465923074785/gfor-20230331xs4a.htm). Amyloid Beta 42 (Aβ42), Aβ42/Aβ40 ratio, tau proteins which includes total tau (t-tau) and phosphorylated tau (p-tau 181), Neurofilament light (NfL), cytokine, chemokine for Fractalkine (CX3CL1), Glial fibrillary acidic protein (GFAP) and Chitinase-3-like protein 1 (YKL-40) were evaluated.

The quantification of the markers was conducted using Meso Scale Discovery (MSD) multiplexed sandwich immunoassays. MSD assays are designed to measure levels of peptide and protein in biological samples. The multiplexed assays use electrochemiluminescent labels that are conjugated to detection antibodies. The labels allow for ultra-sensitive detection. Analytes in the sample bind to capture antibodies immobilized on the working electrode surface and recruitment of the detection antibodies conjugated with electrochemiluminescent labels. Electricity is applied to the electrodes by an MSD instrument leading to light emission by the conjugated labels. Light intensity is then measured to quantify analytes in the sample.

To identify the Maximum Tolerated Dose (MTD) of SNK01, the subjects were given SNK01 in an open-label setting per the following treatment plan, using a 3+3 design (Table 7):
Cohort 1—1.0×10⁹ cells; infusions Q3W, total of four doses.
Cohort 2—2.0×10⁹ cells; infusions Q3W, total of four doses.
Cohort 3—4.0×10⁹ cells; infusions Q3W, total of four doses.

TABLE 7

| Cohort | SNK 01 Dosing | Subject ID |
|---|---|---|
| Cohort 1 | 1.0 × 10⁹ cells (4 doses Q3W) | MX04-201-002 |
| | | MX04-201-003 |
| | | MX04-201-004 |
| | | MX04-201-005 |
| Cohort 2 | 2.0 × 10⁹ cells (4 doses Q3W) | MX04-201-006 * |
| | | MX04-201-007 |
| | | MX04-201-011 |
| Cohort 3 | 4.0 × 10⁹ cells (4 doses Q3W) | MX04-201-012** |
| | | MX04-201-014*** |
| | | MX04-201-015*** |

* MX04-201-006: No cognitive test is available at visit 6
**MX04-201-012: No assessment for EOT visit 6
***MX04-201-014, MX04-201-015 - study in progress, cognitive and lab assessment results are not available as of Apr. 21, 2023.

Once the MTD was identified, a final cohort, Cohort 4, including 12 subjects, received the MTD to study safety, tolerability, and preliminary efficacy

Subjects were assessed at visit 1 to establish a baseline prior to the first dose of SNK01. Table 8 shows the mean and median baseline profile of subjects prior to the first dose of SNK01.

TABLE 8

| Profile | | Mean (min, max) | | Median (IQR) | |
|---|---|---|---|---|---|
| N | | 8 | | 8 | |
| Age (year) | | 76 | (56, 84) | 79 | (72.5, 79.8) |
| Cognitive | CDR (0-3) | 1.69 | (0.5, 3) | 1.5 | (1.0, 2.75) |
| Assessments | ADAS-Cog (0-80) | 39.38 | (18, 75) | 37.5 | (30.25, 42.5) |
| | MMSE (0-30) | 14.88 | (2, 23) | 14 | (13, 20.5) |
| CSF Biomarkers and | Aβ-42 (pg/mL) | 326.5 | (198.4, 503.6) | 285.9 | (251.3, 443.7) |
| Neuroinflammatory | Aβ-42/40 | 0.0458 | (0.0256, 0.0642) | 0.0455 | (0.0404, 0.0541) |
| markers | Total Tau | 900.30 | (435.47, 1540.21) | 766.58 | (616.78, 1350.12) |
| | (pg/mL) | | | | |
| | pTau 181 | 75.27 | (44.48, 177) | 54.72 | (48.71, 95.05) |
| | (pg/mL) | | | | |
| | GFAP (pg/mL) | 5662.58 | (2675.69, 9280.46) | 5586.93 | (4605.04, 6466.42) |
| | NfL (pg/mL) | 12255.90 | (5367.99, 22660.66) | 12355.65 | (7125.37, 14642.03) |
| | YKL-40 | 250019.90 | (145735.11, 452943.86) | 207509.66 | (159828.79, 341704.72) |
| | (pg/mL) | | | | |
| | CX3CL1 | 1796.04 | (1313.36, 2243.24) | 1788.78 | (1495.55, 2156.21) |
| | (Fractalkine) | | | | |
| | (pg/mL) | | | | |
| | TNF-α (pg/mL) | 0.246 | (0.16, 0.32) | 0.24 | (0.193, 0.300) |
| | IL-6 (pg/mL) | 1.49 | (0.75, 3.04) | 1.11 | (0.78, 2.24) |
| | IL-8 (pg/mL) | 55.54 | (43.33, 71.6) | 56.06 | (47.09, 60.53) |
| | IL-12/IL-23p40 | 6.14 | (4.11, 9.14) | 5.89 | (4.65, 7.60) |
| | (pg/mL) | | | | |
| | sTREM2 (pg/mL) | 4847 | (2885, 7282) | 4602 | (3673, 6211) |

Table 9 shows a comparison of the baseline profile of study subjects as compared to the profile of Alzheimer's disease subjects as reported in the art.

TABLE 9

| | | | Reported Data | | |
|---|---|---|---|---|---|
| | MX04 | | | | Control |
| CSF | | AD, | | AD, | (Healthy), |
| Biomarker | Assay Method | Mean ± SD | Assay Method | Mean ± SD | Mean ± SD |
| Aβ-42 | Electrochemiluminescence; | [symbol] ± | Electrochemiluminescence; | 318 ± 95 | 542 ± 239 |
| (pg/mL) | Meso Scale Discovery | 109.4 | Meso Scale Discovery | (n = 50) | (n = 54) |
| | | (n = 8) | | | |
| Aβ-42/40 | Electrochemiluminescence; | 0.0458 ± | Lumipulse fully | 0.0453 ± | 0.0991 ± |
| | Meso Scale Discovery | 0.0114 | automated platform | 0.0108 | 0.0181 |
| | | (n = 8) | (Fujirebio-Europe) | (n = 518) | (n = 197) |
| Total Tau | Electrochemiluminescence; | 900.30 ± | Electrochemiluminescence; | 1331 ± | 636.5 ± |
| (pg/mL) | Meso Scale Discovery | 407 | Meso Scale Discovery | 713.8 | 161.5 |
| | | (n = 8) | | (n = 24) | (n = 18) |
| pTau 181 | Electrochemiluminescence; | 75.27 ± | Electrochemiluminescence; | 76.94 ± | 39.27 ± |
| (pg/mL) | Meso Scale Discovery | 45.3 | Meso Scale Discovery | 33.31 | 23.49 |
| | | (n = 8) | | (n = 98) | (n = 47) |
| GFAP | Electrochemiluminescence; | 5662.58 ± | NA | NA | NA |
| (pg/mL) | Meso Scale Discovery | 1892 | | | |
| | | (n = 8) | | | |
| NF-L | Electrochemiluminescence; | 12255.90 ± | NA | NA | NA |
| (pg/mL) | Meso Scale Discovery | 5541 | | | |
| | | (n = 8) | | | |

[symbol]

indicates data missing or illegible when filed

Subjects were then assessed again at visit 5 (week 11), 1 week after the last dose of SNK01. Subjects were assessed once again at visit 6 (week 22), 12 weeks after the last dose of SNK01.

Assessments included detecting and or quantifying one or more biomarkers, immunophenotyping, genotyping, evaluating NK cell activity, performing one or more cognitive assessments. Alzheimer's Disease biomarkers, pro-inflammatory biomarkers, and anti-inflammatory biomarkers were evaluated in the subjects' CSF and plasma. CSF core biomarkers included amyloid beta 42, amyloid beta 40, amyloid beta 42/40 ratio, total Tau (Tt-tau), phosphorylated Tau (p-Tau), p-Tau 181, and neurofilament light (NfL). CSF inflammatory marker included glial fibrillary acidic protein (GFAP), YKL-40, IL-12/IL-23p40, IL-6, IL-8, TNF-α, IL-10, GM-CSF, IL-1β, and INF-γ. CSF immune cell chemokine ligand included CX3CL1 (Fractalkine). CSF innate immune receptor biomarker included soluble TREM2. Plasma biomarkers included amyloid beta 42, amyloid beta 40, amyloid beta 42/40 ratio, total tau (t-tau), phosphorylated tau (p-tau), Glial Fibrillary Acidic Protein (GFAP), and neurofilament light (NfL). Plasma inflammatory markers included YKL-40, IL-1β, IL-6, IL-8, IL-10, TNF-α, and INF-γ.

Immunophenotyping was performed on the subjects' CSF and whole blood. SNP genotyping of APOE and TREM2. NK cell activity was analyzed using NK Vue. Cognitive assessments included CDR, ADAS-Cog, and MMSE.

Table 10 shows the CDR score of subjects MX04-201-002, MX04-201-003, MX04-201-004, MX04-201-005, MX04-201-006, MX04-201-007, MX04-201-011, and MX04-201-012, at baseline, week 11, and week 22 of the study.

TABLE 10
CDR (Clinical Dementia Rating) - Five-point scale (0, 0.5, 1, 2, 3)

| Analysis | MX04-201- | MX04-201- | MX04-201- | MX04-201- | MX04-201- | MX04-201- | MX04-201- | MX04-201- |
|---|---|---|---|---|---|---|---|---|
| Visit, week | 002 | 003 | 004 | 005 | 006 | 007 | 011 | 012 |
| 0 | 2 | 2 | 1 | 1 | 3 | 1 | 0.5 | 3 |
| 11 | 3 | 2 | 1 | 0.5 | 3 | 0.5 | 0.5 | 3 |
| 22 | 3 | 2 | 2 | 0.5 | | 1 | 0.5 | |

CDR scale of 0-3: no dementia (CDR = 0), questionable dementia (CDR = 0.5), MCI (CDR = 1), moderate cognitive impairment (CDR = 2), and severe cognitive impairment (CDR = 3).

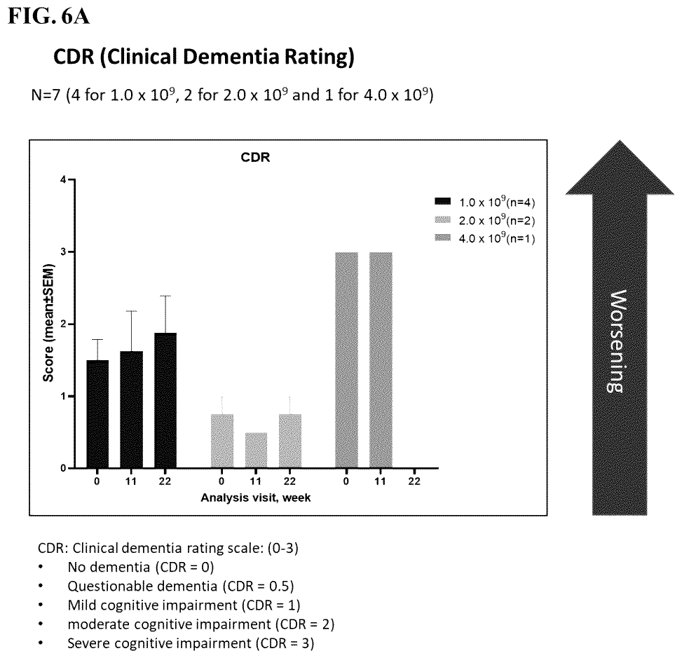

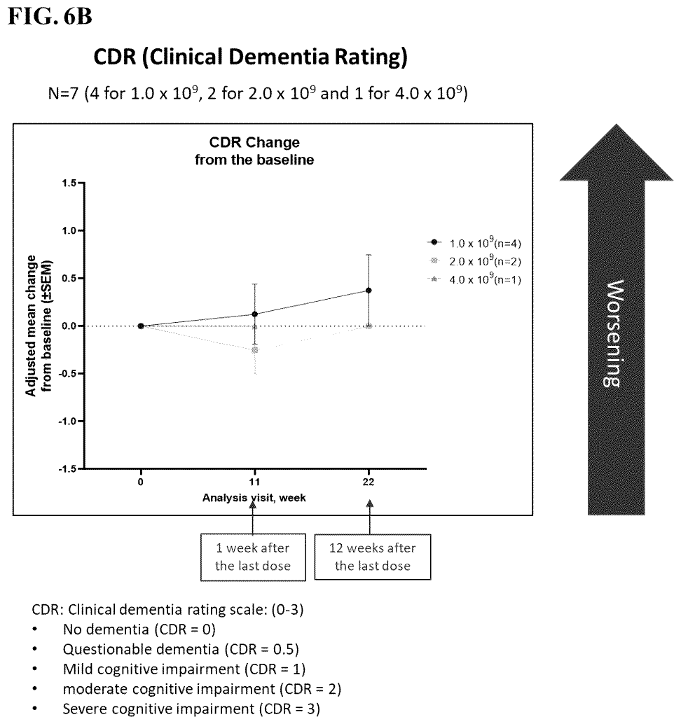

FIG. 6A is a bar graph depicting the Clinical Dementia Rating (CDR) score of subjects treated with NK cells.

FIG. 6B is a line graph depicting the change in Clinical Dementia Rating (CDR) score of subjects treated with NK cells.

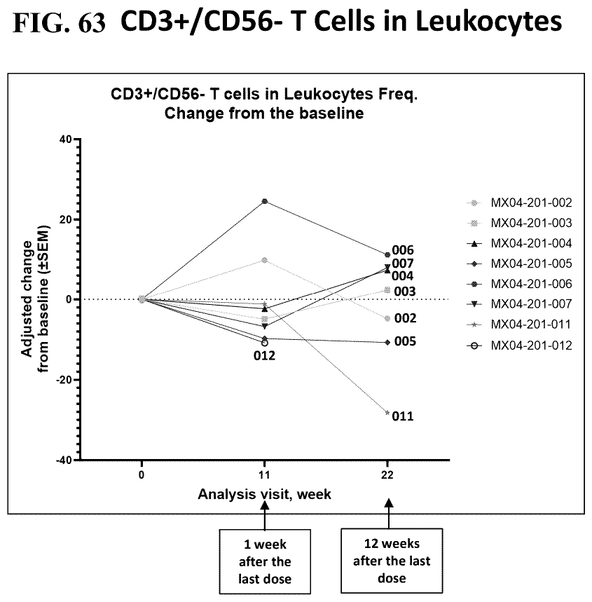

As can be seen in Table 10, FIG. 6A, and FIG. 63, administration of SNK01 to AD subjects, may decrease the subject's CDR score.

Table 11 shows the ADAS-Cog scores of subjects MX04-201-002, MX04-201-003, MX04-201-004, MX04-201-005, MX04-201-006, MX04-201-007, MX04-201-011, and MX04-201-012, at baseline, week 11, and week 22 of the study.

TABLE 11
ADAS-Cog (Alzheimer's Disease Assessment Scale-Cognitive subscale) - Scale range 0 to 80

| Analysis Visit, | MX04-201- | MX04-201- | MX04-201- | MX04-201- | MX04-201- | MX04-201- | MX04-201- | MX04-201- |
|---|---|---|---|---|---|---|---|---|
| week | 002 | 003 | 004 | 005 | 006 | 007 | 011 | 012 |
| 0 | 43 | 41 | 38 | 34 | 37 | 29 | 18 | 75 |
| 11 | 44 | 44 | 36 | 29 | 64 | 28 | 32 | 78 |
| 22 | 36 | 43 | 37 | 33 | | 27 | 30 | |

The greater the dysfunction, the greater the score. A score of 80 represents the most severe impairment and 0 represents the least impairment.

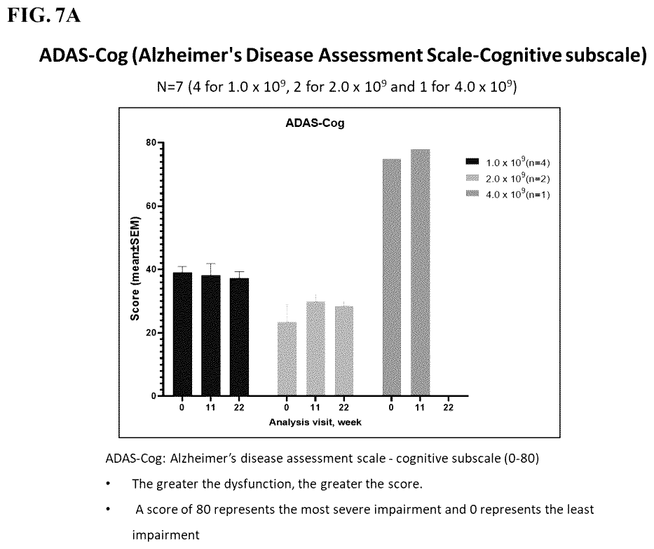

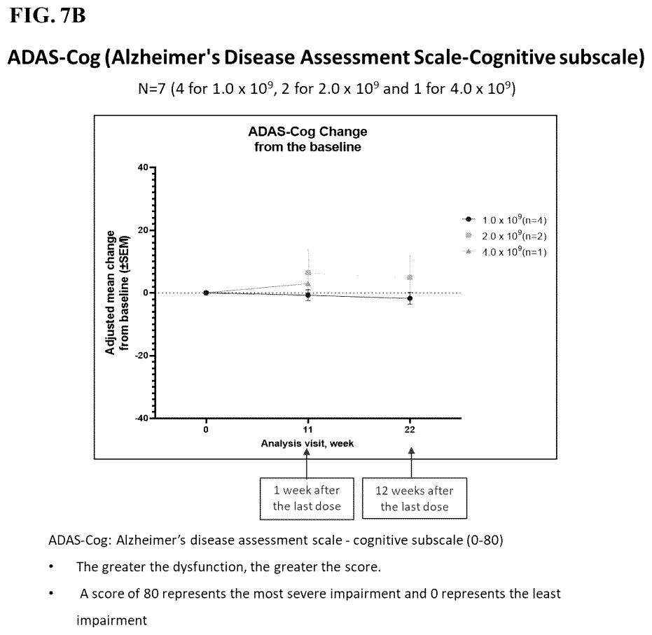

FIG. 7A is a bar graph depicting the Alzheimer's disease assessment scale-cognitive subscale (ADAS-Cog) scores of subjects treated with NK cells.

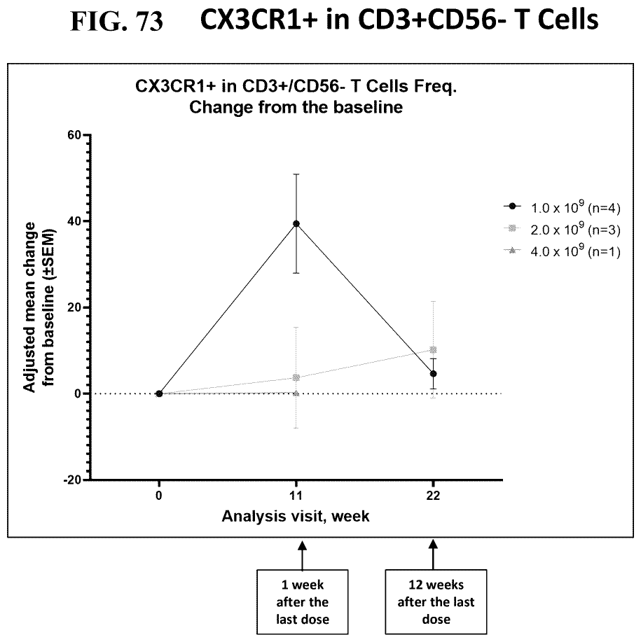

FIG. 73 is a line graph depicting the change in Alzheimer's disease assessment scale-cognitive subscale (ADAS-Cog) scores of subjects treated with NK cells.

As can be seen in Table 11, FIG. 7A, and FIG. 7, administration of SNK01 to AD subjects, may decrease the subject's ADAS-Cog score.

Table 12 shows the CDR score of subjects MX04-201-002, MX04-201-003, MX04-201-004, MX04-201-005, MX04-201-006, MX04-201-007, MX04-201-011, and MX04-201-012, at baseline, week 11, and week 22 of the study.

TABLE 12
MMSE (Mini-Mental State Examination) - Scale 0-30

| Analysis | MX04-201- | MX04-201- | MX04-201- | MX04-201- | MX04-201- | MX04-201- | MX04-201- | MX04-201- |
|---|---|---|---|---|---|---|---|---|
| Visit, week | 002 | 003 | 004 | 005 | 006 | 007 | 011 | 012 |
| 0 | 14 | 13 | 13 | 23 | 14 | 19 | 21 | 2 |
| 11 | 11 | 13 | 12 | 19 | 11 | 17 | 20 | 1 |
| 22 | 11 | 17 | 13 | 16 | | 17 | 20 | |

No cognitive impairment 24-30; mild cognitive impairment 19-23; moderate cognitive impairment 10-18; and severe cognitive impairment ≤9.

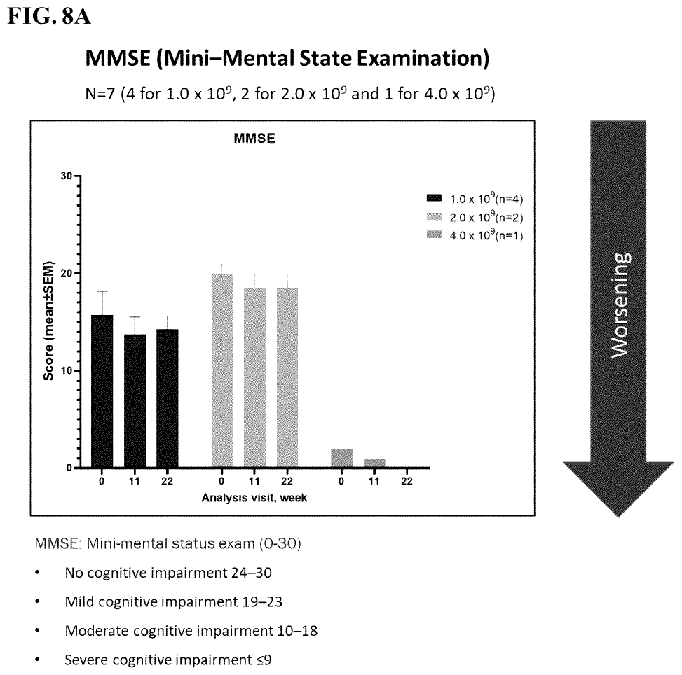

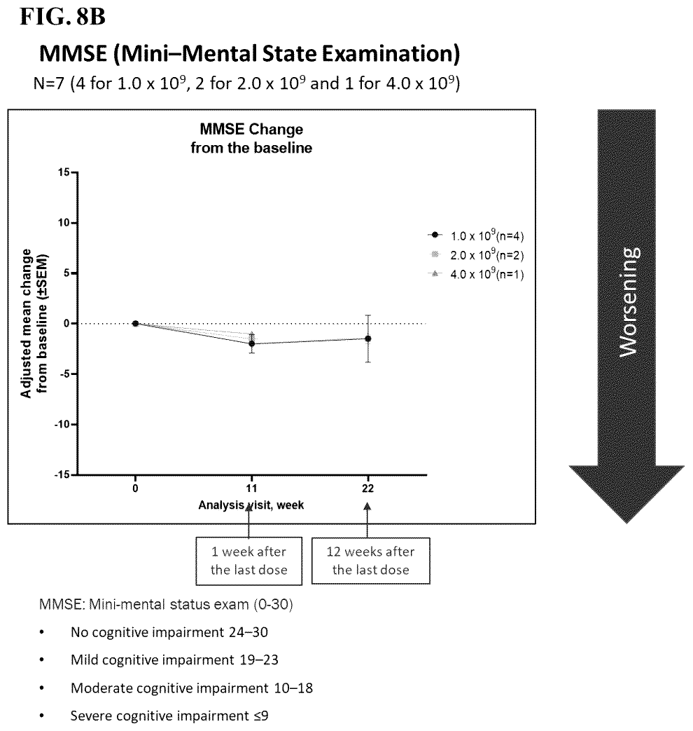

FIG. 8A is a bar graph depicting the Mini-Mental State Examination (MMSE) scores of subjects treated with NK cells.

FIG. 813 is a line graph depicting the Mini-Mental State Examination (MMSE) scores of subjects treated with NK cells.

As can be seen in Table 12, FIG. 8A, and FIG. 8B, administration of SNK01 to AD subjects, may increase the subject's MMSE score.

Table 13 shows the mean change in CDR score, ADAS-Cog score, and MMSE score from baseline at week 11 and week 22 of the study.

TABLE 13

| | Week 11 Mean Change from the baseline | | | | | | Week 22 Mean Change from the baseline | | | |
|---|---|---|---|---|---|---|---|---|---|---|
| Preliminary | 1.0 × 10⁹ | | 2.0 × 10⁹ | | 4.0 × 10⁹ | | 1.0 × 10⁹ | | 2.0 × 10⁹ | |
| Endpoint | n = 4 | | n = 2 | | n = 1 | | n = 4 | | n = 2 | |
| CDR | 0.13 | (8.3%) | −0.25 | (33.3%) | 0.00 | (0.0%) | 0.38 | (25.0%) | 0.00 | (0.0%) |
| ADAS-Cog | −0.75 | (−1.9%) | 6.50 | (27.7%) | 3.00 | (4.0%) | −1.75 | (−4.5%) | 5.00 | (21.3%) |
| MMSE | −2.00 | (−12.7%) | −1.50 | (−7.5%) | −1.00 | (−50%) | −1.50 | (−9.5%) | −1.50 | (−7.5%) |

CDR: Decrease = Improvement
ADAS-Cog: Decrease = Improvement
MMSE: Increase = Improvement

As can be seen in Table 13, administration of 1.0×10⁹-4.0×10⁹ SNK01, may improve or stabilize the CDR score, ADAS-Cog score, and MMSE score, of the subjects.

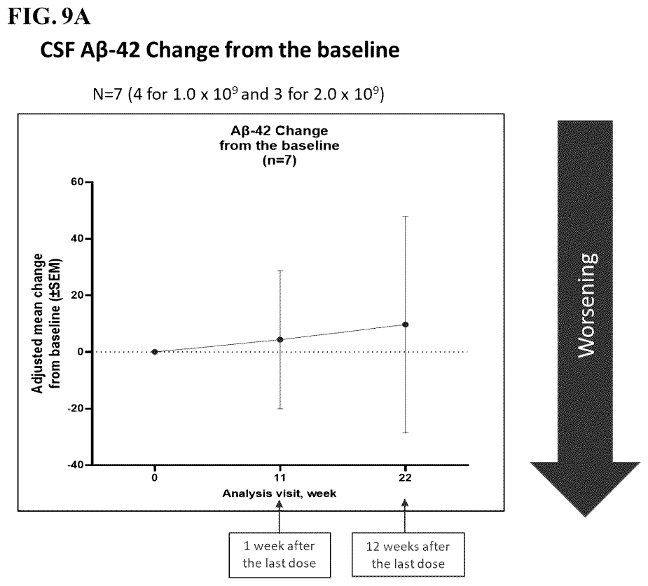

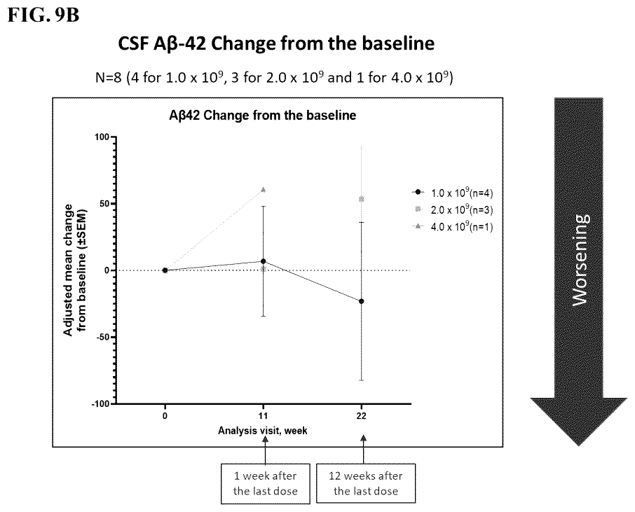

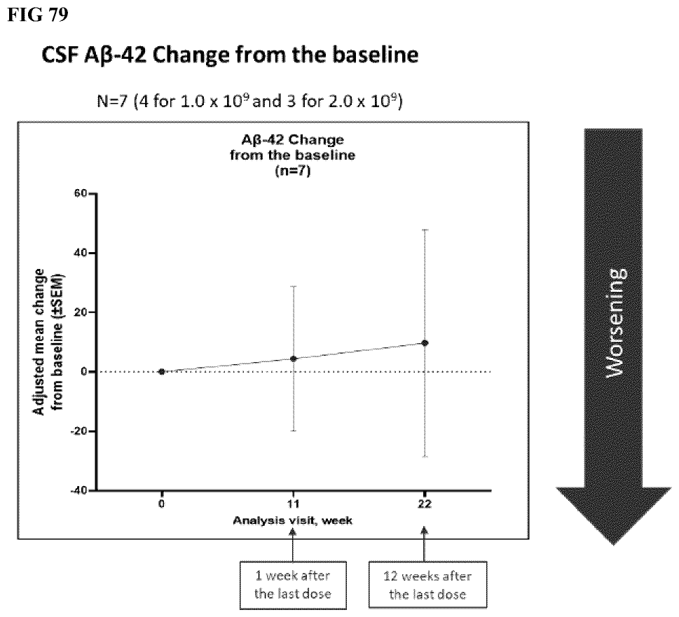

FIG. 9A is a line graph depicting the average change in Aβ-42 levels in the cerebrospinal fluid of subjects treated with different doses of NK cells. FIG. 79 is the same as FIG. 9A.

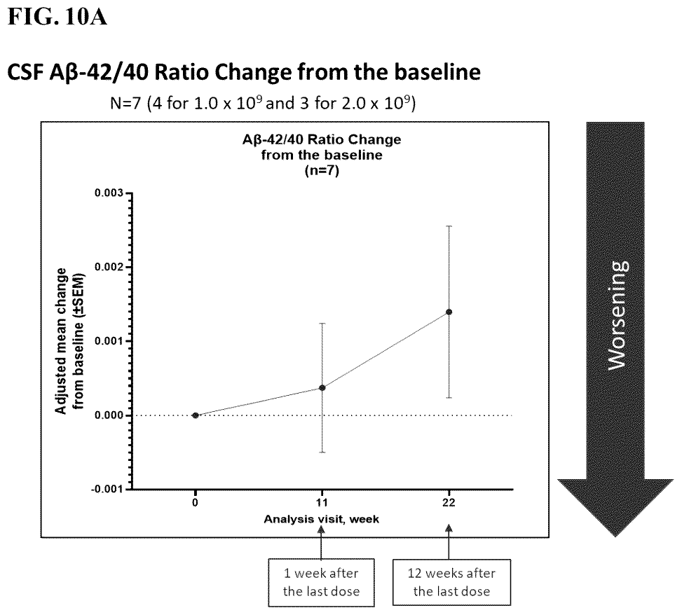

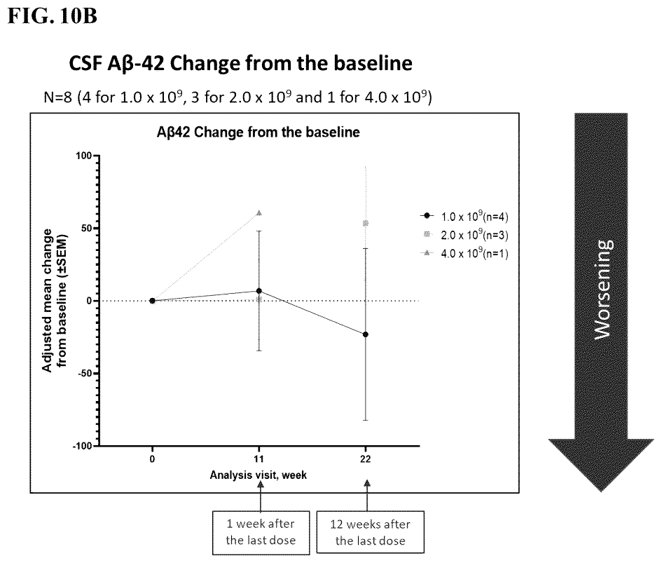

FIG. 9B is a line graph depicting the change in Aβ-42 levels in the cerebrospinal fluid of subjects treated with different doses of NK cells. FIG. 10B is the same as FIG. 9B.

As can be seen in FIG. 9A and FIG. 9B, Aβ-42 levels increase in the cerebrospinal fluid of subjects treated with NK cells.

FIG. 10A is a line graph depicting the aggregate change in Aβ-42/40 ratio in the cerebrospinal fluid of subjects treated with different doses of NK cells.

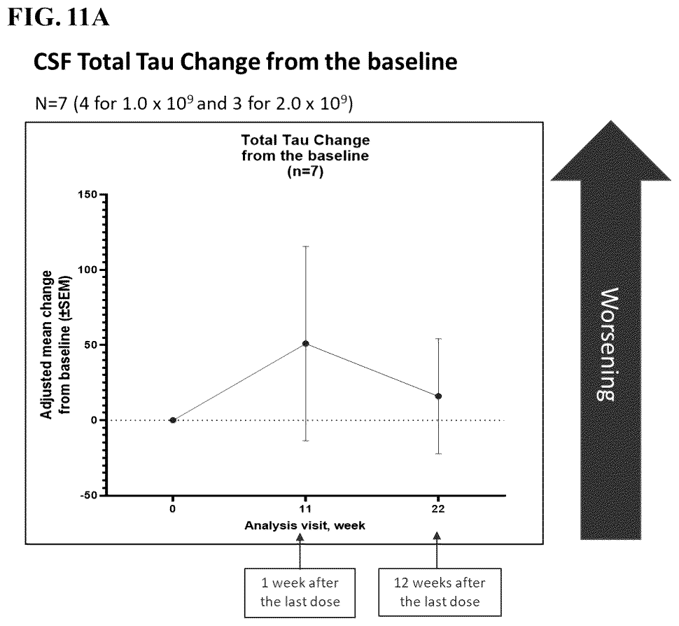

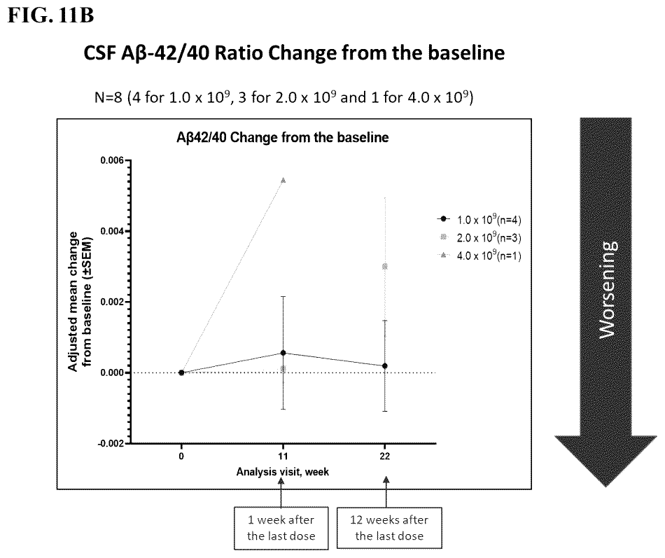

FIG. 11B is a line graph depicting the change in Aβ-42/40 ratio in the cerebrospinal fluid of subjects treated with different doses of NK cells.

As can be seen in FIG. 10A and FIG. 11B, Aβ-42/40 ratio increase in the cerebrospinal fluid of subjects treated with NK cells.

FIG. 11A is a line graph depicting the average change in total Tau levels in the cerebrospinal fluid of subjects treated with different doses of NK cells.

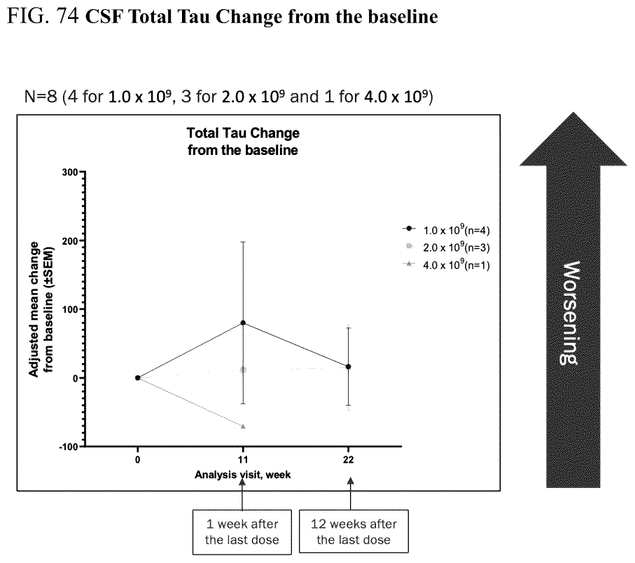

FIG. 74 is a line graph depicting the change in total tau levels in the cerebrospinal fluid of subjects treated with different doses of NK cells.

As can be seen in FIGS. 11A and 74, total tau levels may decrease in the cerebrospinal fluid of subjects treated with NK cells.

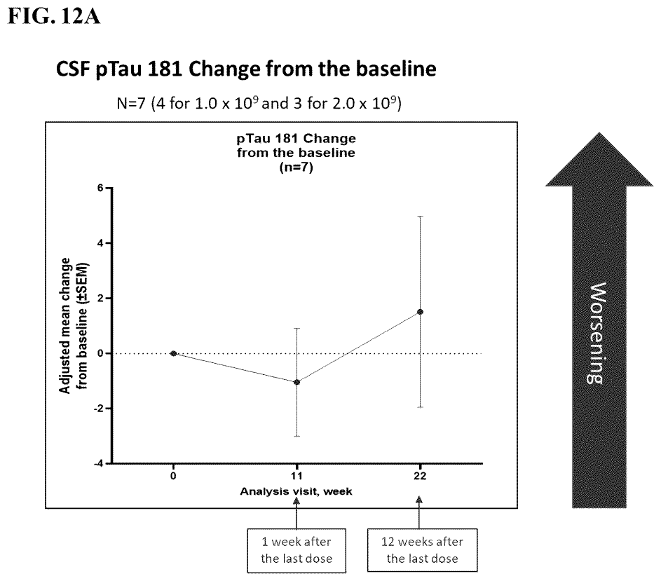

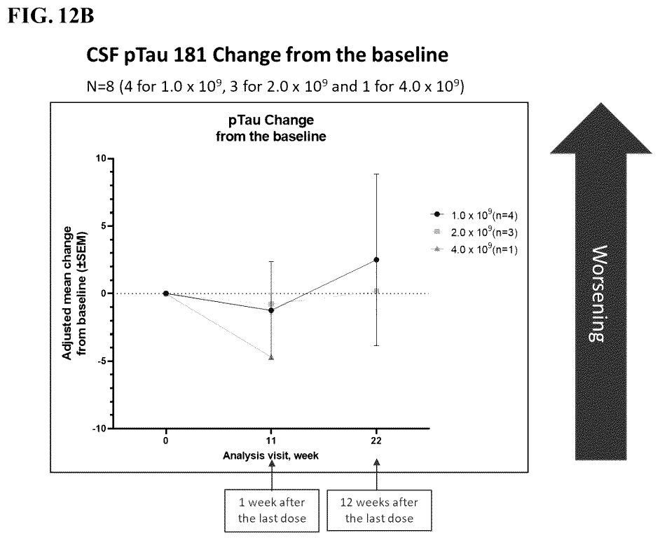

FIG. 12A is a line graph depicting the average change in p-tau 181 levels in the cerebrospinal fluid of subjects treated with different doses of NK cells.

FIG. 12B is a line graph depicting the change in p-tau levels in the cerebrospinal fluid of subjects treated with different doses of NK cells.

As can be seen in FIG. 12A and FIG. 12B, p-tau levels may decrease in the cerebrospinal fluid of subjects treated with NK cells.

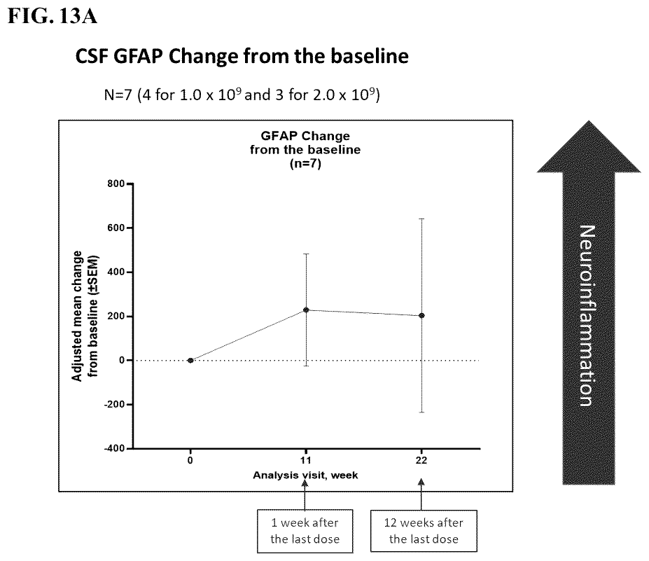

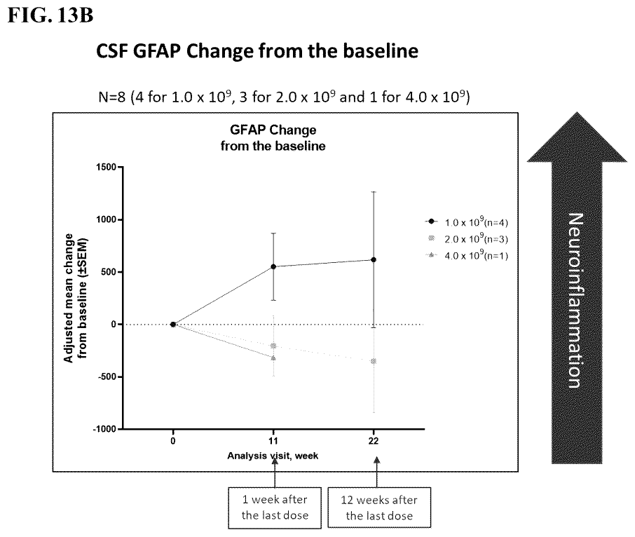

FIG. 13A is a line graph depicting the average change in GFAP levels in the cerebrospinal fluid of subjects treated with different doses of NK cells.

FIG. 13B is a line graph depicting the change in GFAP levels in the cerebrospinal fluid of subjects treated with different doses of NK cells.

As can be seen in FIG. 13A and FIG. 13B, total neuroinflammation, as indicated by GFAP levels in the cerebrospinal fluid of subjects treated with NK cells, may decrease.

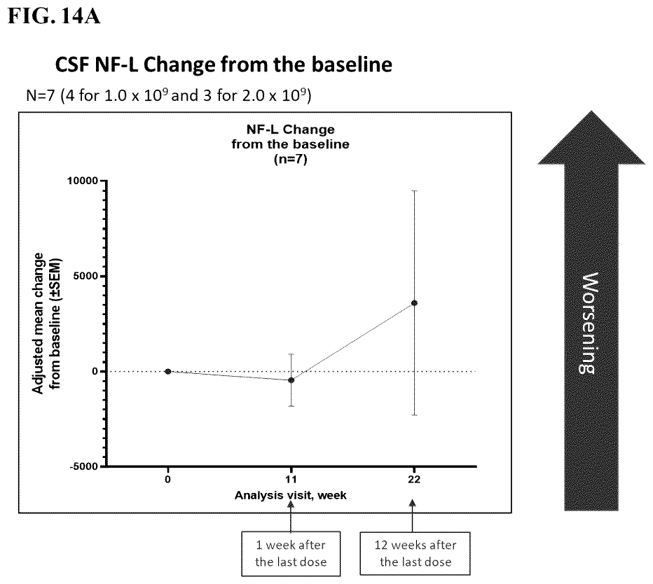

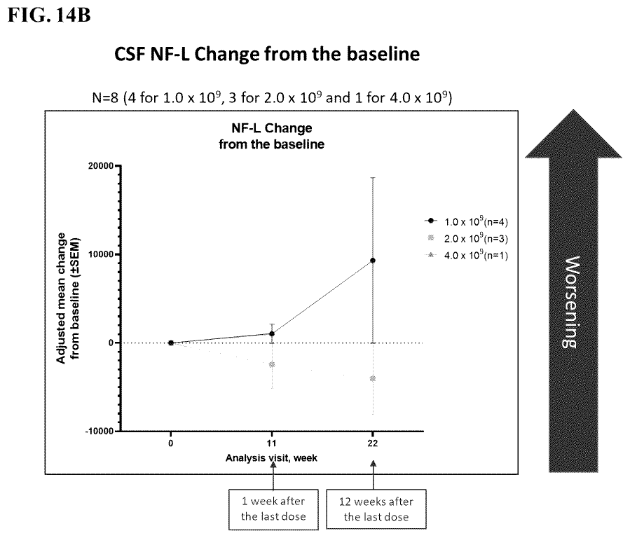

FIG. 14A is a line graph depicting the average change in NfL levels in the cerebrospinal fluid of subjects treated with different doses of NK cells.

FIG. 14B is a line graph depicting the change in NfL levels in the cerebrospinal fluid of subjects treated with different doses of NK cells.

As can be seen in FIG. 14A and FIG. 14B, NfL levels may decrease in the cerebrospinal fluid of subjects treated with NK cells.

Table 14 shows the mean change in Aβ-42/40, Aβ-42, total tau, p-tau, GFAP, and NfL, from baseline at week 11 and week 22 of the study.

TABLE 14

| | Week 11 Mean Change from the baseline | | | | | | Week 22 Mean Change from the baseline | | | |
|---|---|---|---|---|---|---|---|---|---|---|
| | 1.0 × 10⁹ | | 2.0 × 10⁹ | | 4.0 × 10⁹ | | 1.0 × 10⁹ | | 2.0 × 10⁹ | |
| | n = 4 | | n = 3 | | n = 1 | | n = 4 | | n = 3 | |
| Aβ-42/40 | 0.00056 | (1.2%) | 10.00012 | (0.3%) | 0.00545 | (11.3%) | 0.00019 | (0.4%) | 0.003 | (7.1%) |
| Aβ-42 | 6.85 | (2.1%) | 0.99 | (0.3%) | 60.89 | (23.2%) | −23.22 | (−7.0%) | 53.48 | (15.6%) |
| Total tau | 80.13 | (7.9%) | 12.06 | (1.5%) | −70.42 | (−10.0%) | 16.28 | (1.6%) | 15.43 | (1.9%) |
| p-tau 181 | −1.25 | (−1.3%) | −0.77 | (−1.4%) | −4.68 | (−8.2%) | 2.50 | (2.6%) | 0.20 | (0.4%) |
| GFAP | 551.90 | (11.6%) | −202.01 | (−3.1%) | −314.18 | (−4.8%) | 618.03 | (13.0%) | −349.24 | (−5.3%) |
| NF-L | 1016.93 | (10.1%) | −2428.38 | (−17.0%) | 1114.30 | (7.6%) | 9306.52 | (92.2%) | −4019.38 | (−28.1%) |

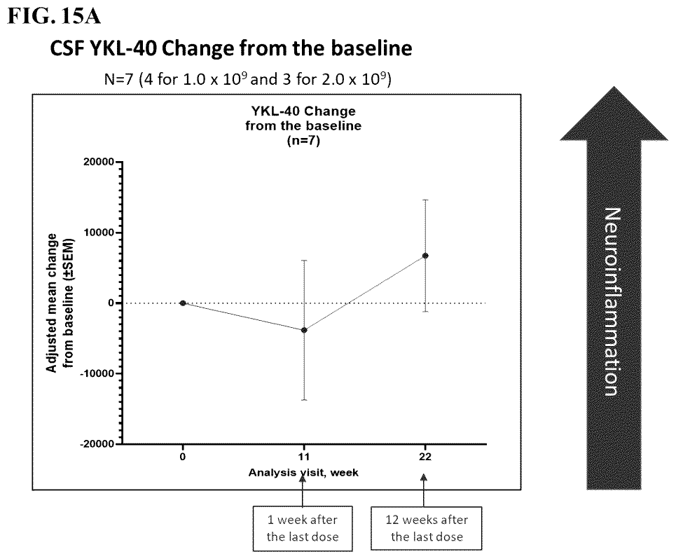

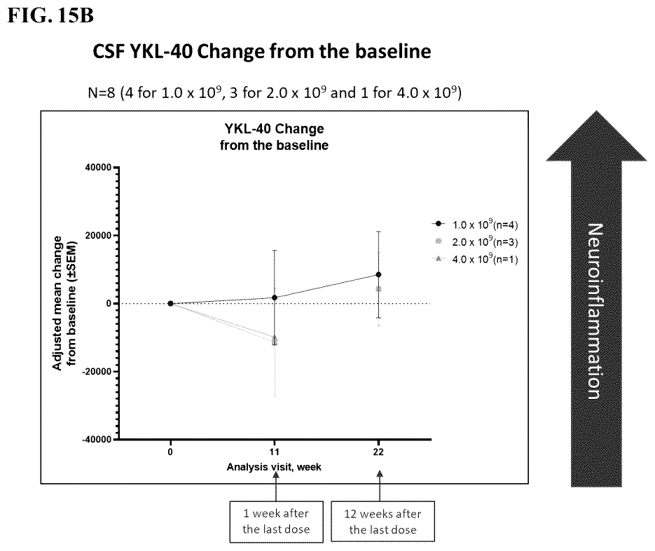

FIG. 15A is a line graph depicting the aggregate change in YKL-40 levels in the cerebrospinal fluid of subjects treated with different doses of NK cells.

FIG. 15B is a line graph depicting the change in YKL-40 levels in the cerebrospinal fluid of subjects treated with different doses NK cells.

As can be seen in FIG. 15A and FIG. 15B, neuroinflammation, as indicated by YKL-40 levels in the cerebrospinal fluid of subjects treated with NK cells, may change from baseline following NK cell administration.

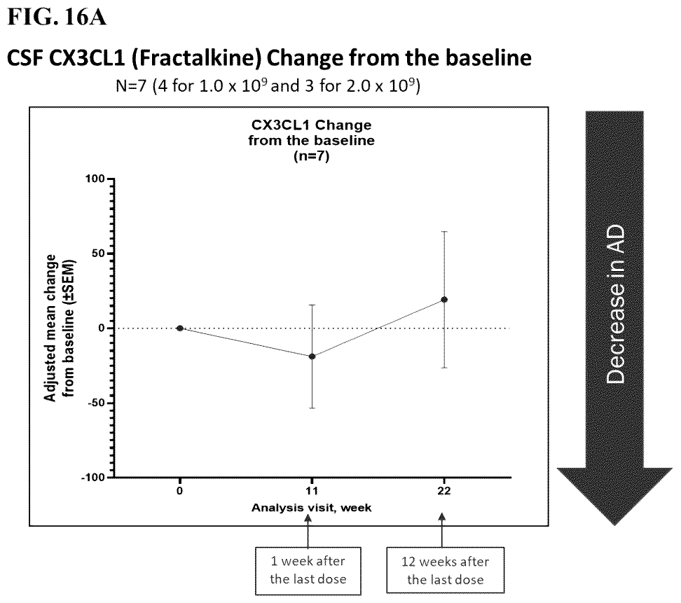

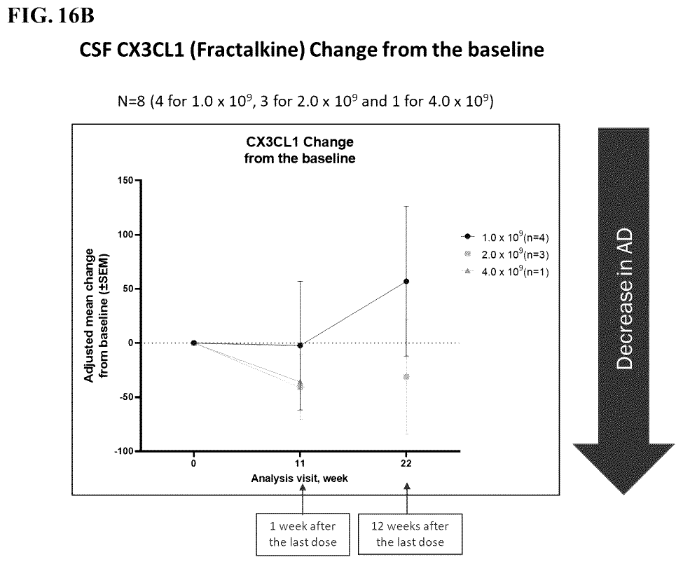

FIG. 16A is a line graph depicting the aggregate change in baseline CX3CL1 (Fractalkine) levels in the cerebrospinal fluid of subjects treated with different doses of NK cells.

FIG. 16B is a line graph depicting the change in baseline CX3CL1 (Fractalkine) levels in the cerebrospinal fluid of subjects treated with different doses NK cells.

As can be seen in FIG. 16A and FIG. 16B, CX3CL1 (Fractalkine) levels in the cerebrospinal fluid of subjects treated with NK cells may decrease from baseline following NK cell administration.

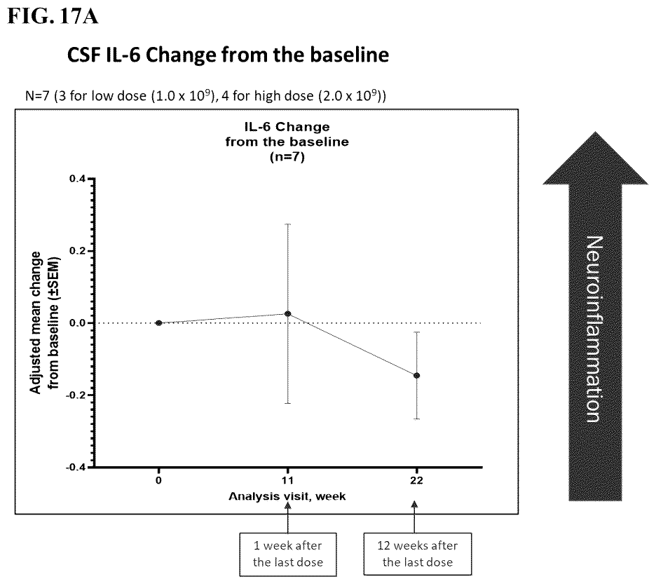

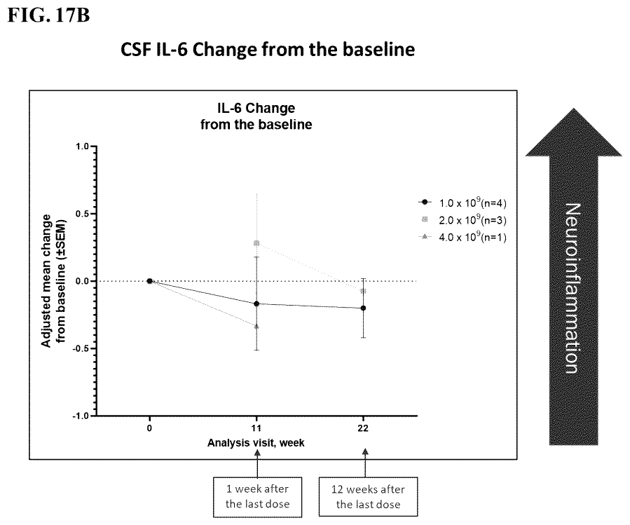

FIG. 17A is a line graph depicting the average change in baseline IL-6 levels in the cerebrospinal fluid of subjects treated with different doses of NK cells.

FIG. 17B is a line graph depicting the change in baseline IL-6 levels in the cerebrospinal fluid of subjects treated with different doses of NK cells.

As can be seen in FIG. 17A and FIG. 17B, IL-6 levels in the cerebrospinal fluid of subjects treated with NK cells may decrease from baseline following NK cell administration.

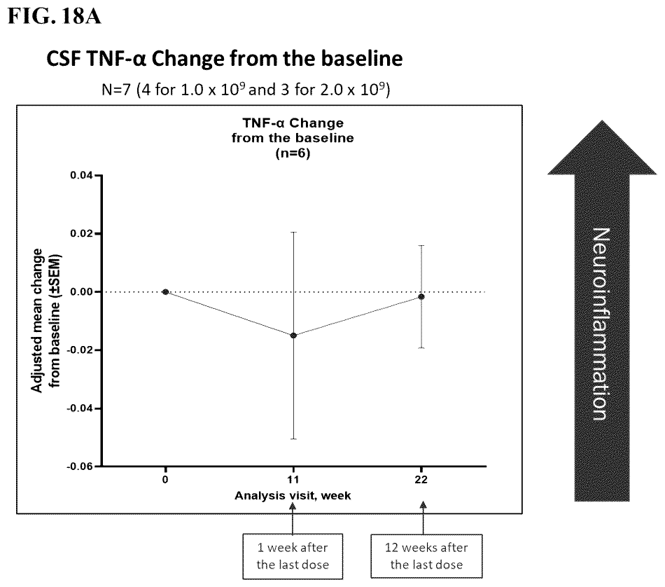

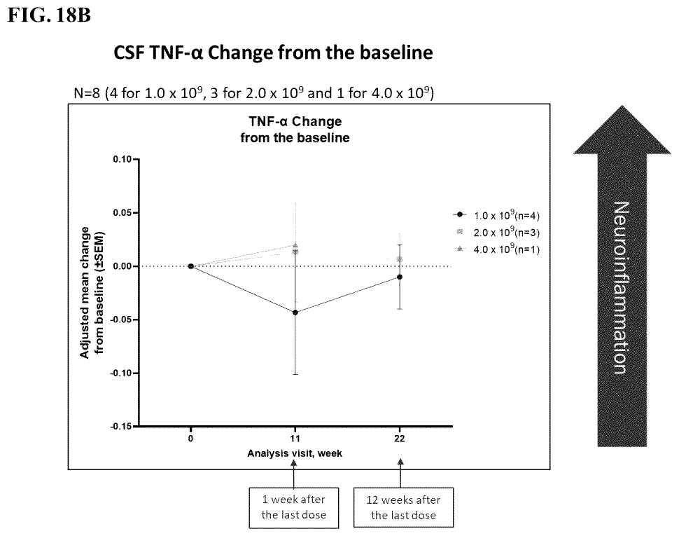

FIG. 18A is a line graph depicting the average change in baseline TNF-α levels in the cerebrospinal fluid of subjects treated with different doses of NK cells.

FIG. 18B is a line graph depicting the change in baseline TNF-α levels in the cerebrospinal fluid of subjects treated with different doses of NK cells.

As can be seen in FIG. 18A and FIG. 18B, TNF-α levels in the cerebrospinal fluid of subjects treated with NK cells may decrease from baseline following NK cell administration.

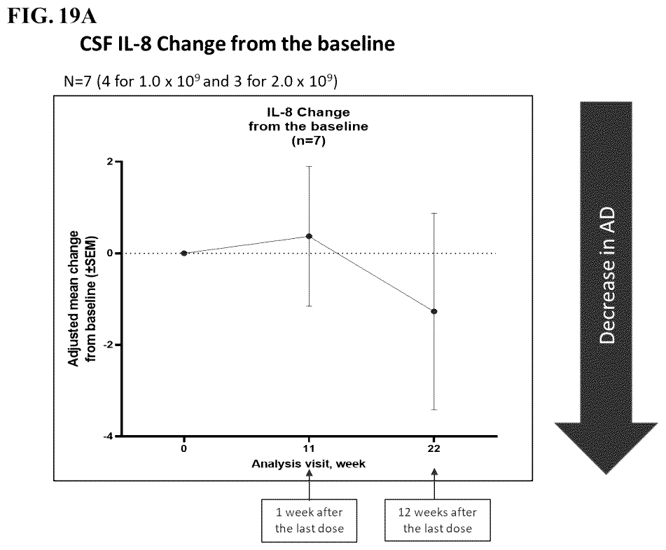

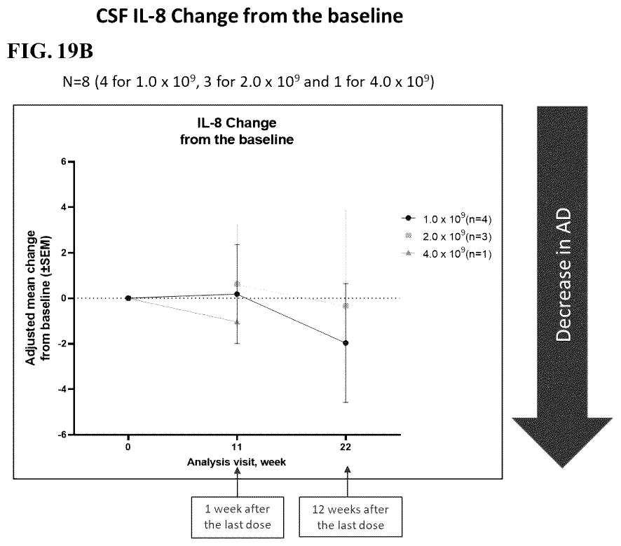

FIG. 19A is a line graph depicting the average change in baseline IL-8 levels in the cerebrospinal fluid of subjects treated with different doses of NK cells.

FIG. 19B is a line graph depicting the change in baseline IL-8 levels in the cerebrospinal fluid of subjects treated with different doses of NK cells.

As can be seen in FIG. 19A and FIG. 19B, IL-8 levels in the cerebrospinal fluid of subjects treated with NK cells may change from baseline following NK cell administration.

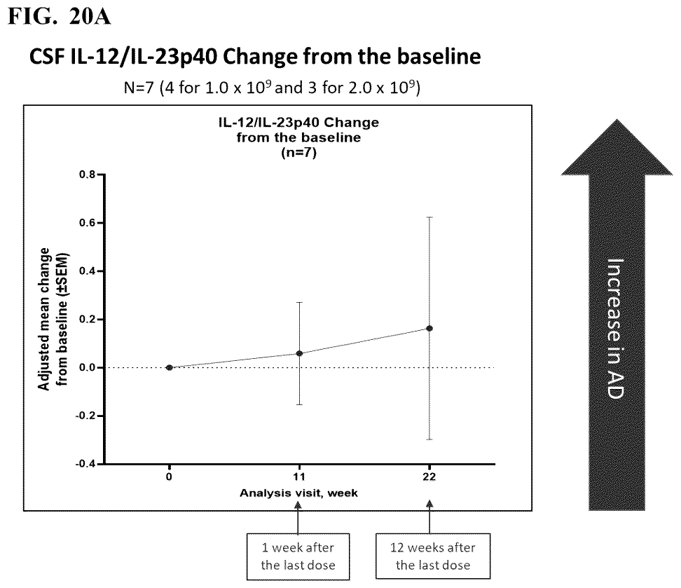

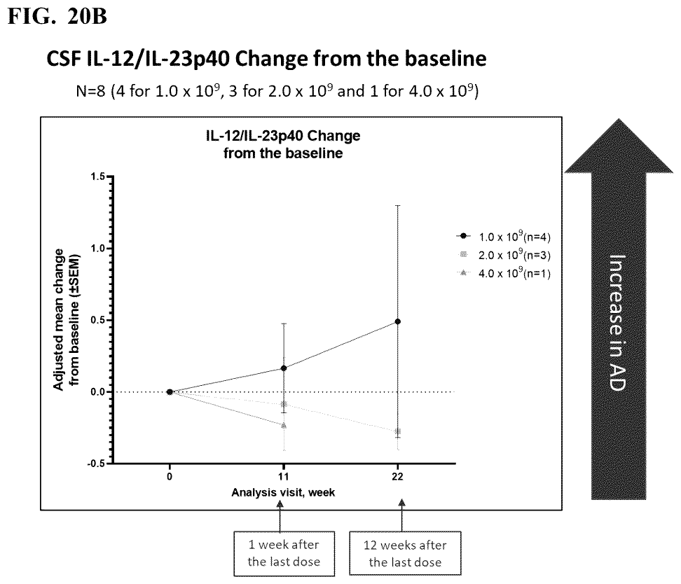

FIG. 20A is a line graph depicting the average change in baseline IL-12/IL-23p40 levels in the cerebrospinal fluid of subjects treated with different doses of NK cells.

FIG. 203 is a line graph depicting the change in baseline IL-12/IL-23p40 levels in the cerebrospinal fluid of subjects treated with different doses of NK cells.

As can be seen in FIG. 20A and FIG. 20, IL-12/IL-23p40 ratio in the cerebrospinal fluid of subjects treated with NK cells may decrease from baseline following NK cell administration.

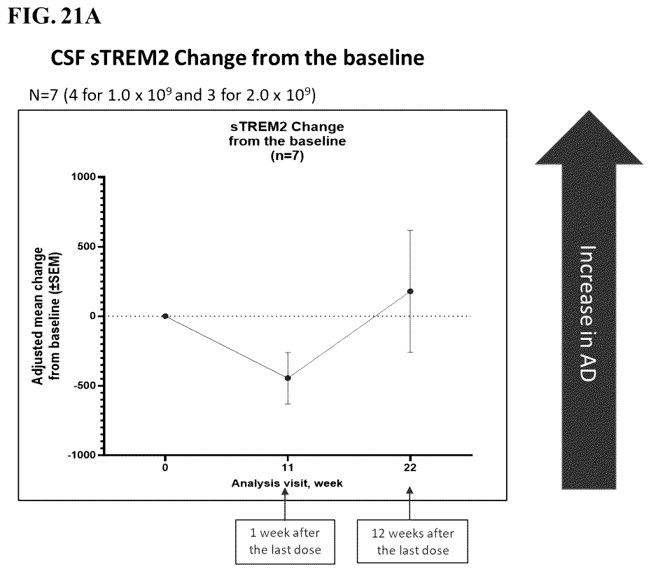

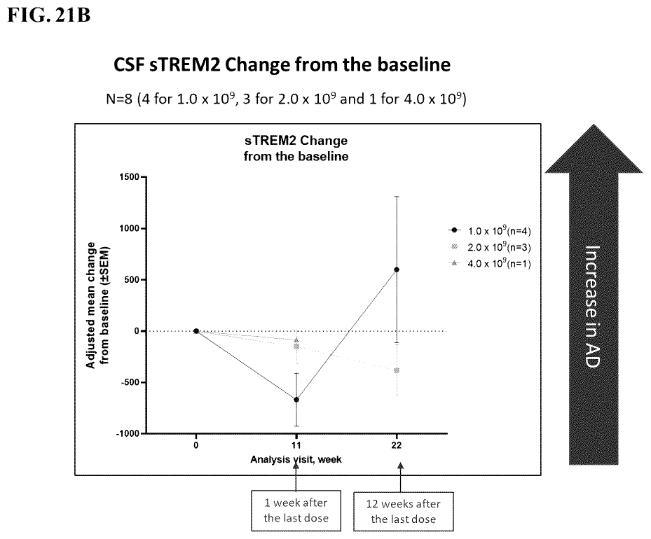

FIG. 21A is a line graph depicting the average change in baseline sTREM2 levels in the cerebrospinal fluid of subjects treated with different doses of NK cells.

FIG. 213 is a line graph depicting the change in baseline sTREM2 levels in the cerebrospinal fluid of subjects treated with different doses of NK cells.

As can be seen in FIG. 21A and FIG. 213, sTREM2 levels in the cerebrospinal fluid of subjects treated with NK cells may decrease from baseline following NK cell administration.

Table 15 shows the mean change in YKL-40, CX3CL1, TNF-αz, IL-6, IL-8, IL-12/IL-23p40, and sTREM2, from baseline at week 11 and week 22 of the study.

TABLE 15

| | Week 11 Mean Change from the baseline | | | | | | Week 22 Mean Change from the baseline | | | |
|---|---|---|---|---|---|---|---|---|---|---|
| CSF | 1.0 × 10⁹ | | 2.0 × 10⁹ | | 4.0 × 10⁹ | | 1.0 × 10⁹ | | 2.0 × 10⁹ | |
| Biomarker | n = 4 | | n = 3 | | n = 1 | | n = 4 | | n = 3 | |
| YKL-40 | 1728.09 | (0.7%) | −11212.80 | (−3.9%) | −9770.55 | (−5.7%) | 8508.31 | (3.6%) | 4345.92 | (1.5%) |
| CX3CL1 | −2.46 | (−0.1%) | −40.86 | (−2.1%) | −35.78 | (−2.0%) | 56.89 | (3.3%) | −31.07 | (−1.6%) |
| TNF-α | −0.04 | (−16.0%) | 0.01 | (5.8%) | 0.02 | (11.1%) | −0.01 | (−5.0%) | 0.01 | (3.1%) |
| IL-6 | −0.16 | (−9.4%) | 0.28 | (28.8%) | −0.33 | (−16.9%) | −0.20 | (−11.4%) | −0.07 | (−7.4%) |
| IL-8 | 0.18 | (0.3%) | 0.62 | (1.2%) | −1.03 | (−1.8%) | −1.97 | (−3.5%) | −0.34 | (−0.6%) |
| IL-12/ | 0.17 | (3.1%) | −0.09 | (−1.2%) | −0.23 | (−4.0%) | 0.49 | (8.9%) | −0.27 | (−3.8%) |
| IL-23p40 | | | | | | | | | | |
| sTREM2 | −667.99 | (−15.9%) | −147.95 | (−3.0) | −83.89 | (−1.2%) | 598.32 | (14.3%) | −381.77 | (−7.8%) |

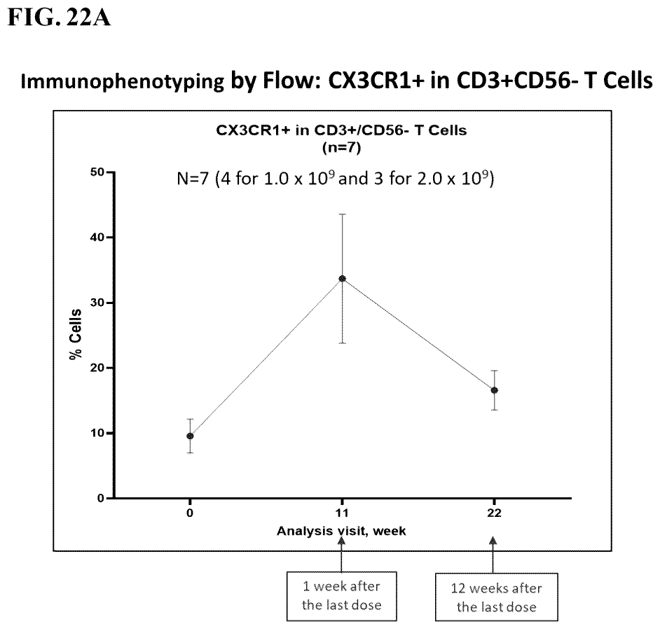

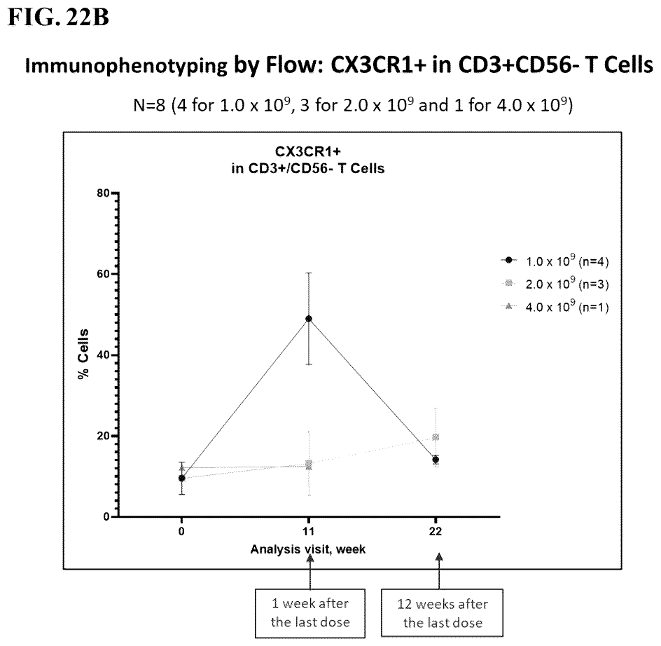

FIG. 22A is a line graph showing the aggregate expression level (percentage) of CX3CR1 in T cells in CSF of subjects treated with different doses of NK cells.

FIG. 22B is a line graph showing the expression level (percentage) of CX3CR1 in T cells in CSF of subjects treated with different doses of NK cells.

As can be seen in FIG. 22A and FIG. 22B, the percentage of CX3CR1+ cells in CD3−CD56+ T-cells may increase following NK cell administration.

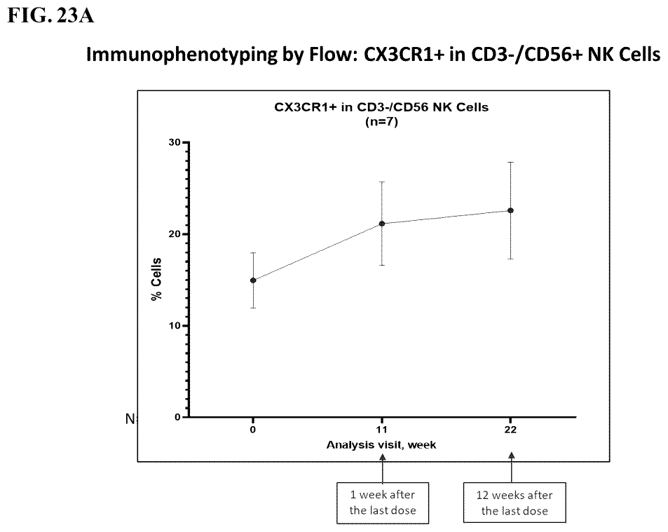

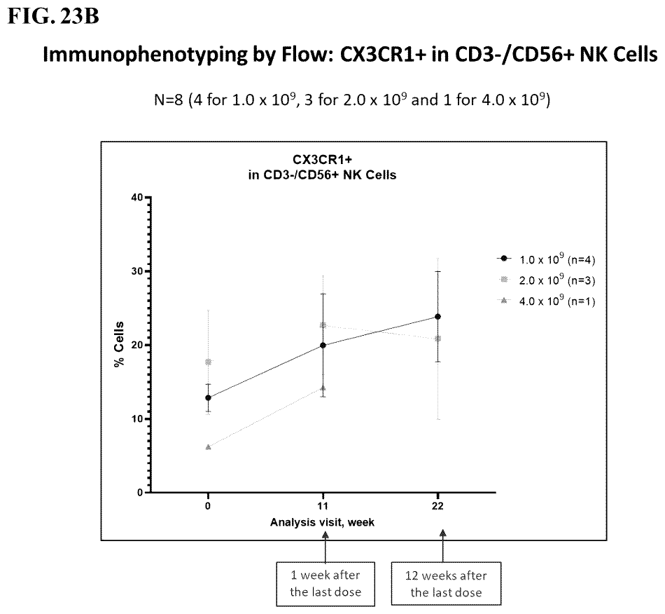

FIG. 23A is a line graph showing the aggregate expression level (percentage) of CX3CR1 in NK cells in CSF of subjects treated with different doses of NK cells.

FIG. 23B is a line graph showing the expression level (percentage) of CX3CR1 in NK cells in CSF of subjects treated with different doses of NK cells.

As can be seen in FIG. 23A and FIG. 23B, the percentage of CX3CR1+ cells in CD3−CD56+ NK cells may increase following NK cell administration.

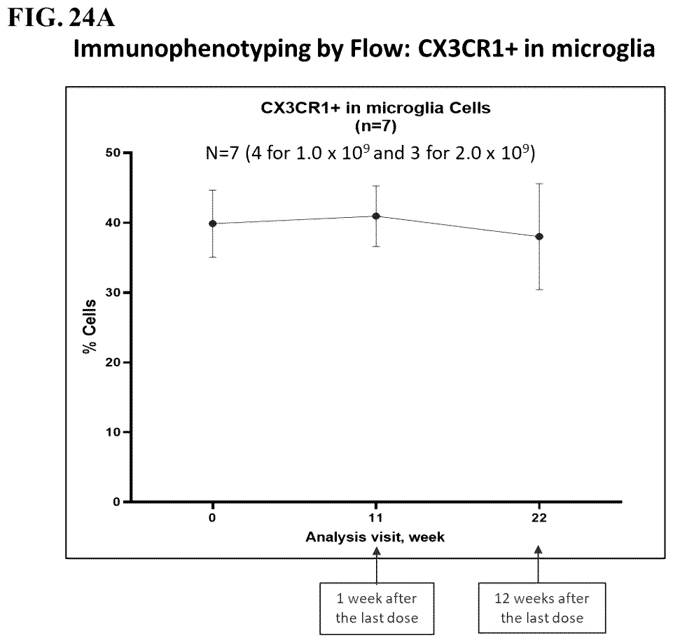

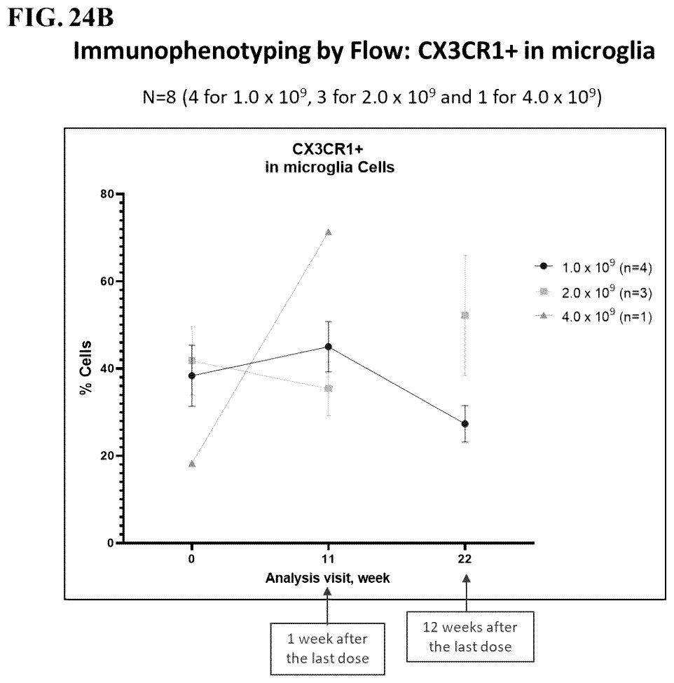

FIG. 24A is a line graph showing the expression level (percentage) of CX3CR1 in microglia in CSF of subjects treated with different doses of NK cells.

FIG. 24B is a line graph showing the expression level (percentage) of CX3CR1 in microglia in CSF of subjects treated with different doses of NK cells.

As can be seen in FIG. 24A and FIG. 24B, the percentage of CX3CR1+ cells in microglia may change following NK cell administration.

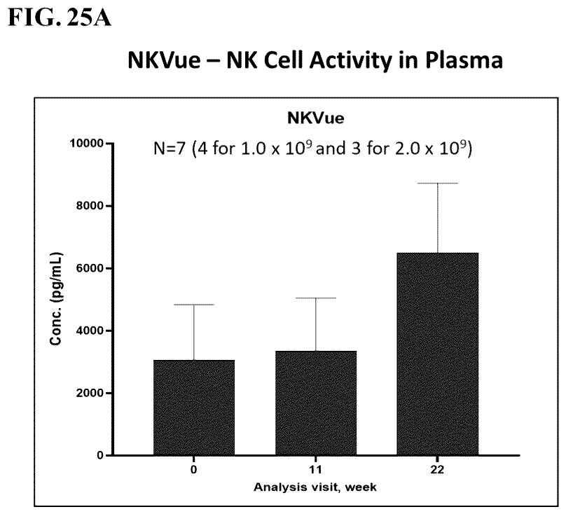

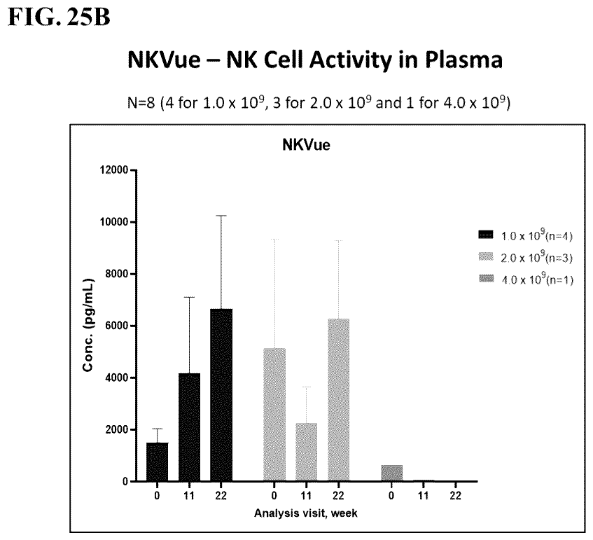

FIG. 25A is a bar graph depicting NK cell activity the plasma of subjects treated with different doses of NK cells.

FIG. 25B is a bar graph depicting NK cell activity in the plasma of subjects treated with different doses of NK cells.

As can be seen in FIG. 25A and FIG. 25B, the NK cell activity may increase following NK cell administration.

### Example 11 Use of Expanded Non-Genetically Modified Natural Killer Cells (SNK01) with Enhanced Cytotoxicity in Patients with Alzheimer's Disease—Interim Report of a Phase I Trial

Purpose/Objectives The accumulation of misfolded proteins is known to elicit a cascade of neuroinflammation by CNS-resident or infiltrating immune cells, resulting in neuronal cell death in Alzheimer's disease (AD). It is now recognized that only clearing these proteins may not be the best treatment strategy for AD.

Natural Killer (NK) cells are an essential part of the innate immune system that have been shown pre-clinically to slow progression of amyloid deposition as well as to decrease neuroinflammation by recognizing and eliminating autoreactive immune cells and damaged neurons. SNK01 is a first-in-kind, autologous non-genetically modified NK cell product with high cytotoxicity and over 90% activating receptor expression. It can be consistently produced from any patients for clinical use. A clinical trial was carried out to try to demonstrate that SNK01 can be safely infused to reduce neuroinflammation by crossing the blood brain barrier (BBB) in AD patients.

Materials & Methods In this Phase 1 dose escalation study (Study SNK01-MX04, NCT04678453), SNK01 was administered intravenously (IV) every three weeks for a total of 4 treatments using a 3+3 dose escalation design [low dose (1×10⁹ cells), medium dose (2×10⁹ cells), and high dose (4×10⁹ cells)] in subjects with either mild, moderate or severe AD confirmed by MRI and PET scans. Assessment of baseline severity was based on the CDR-SB score.

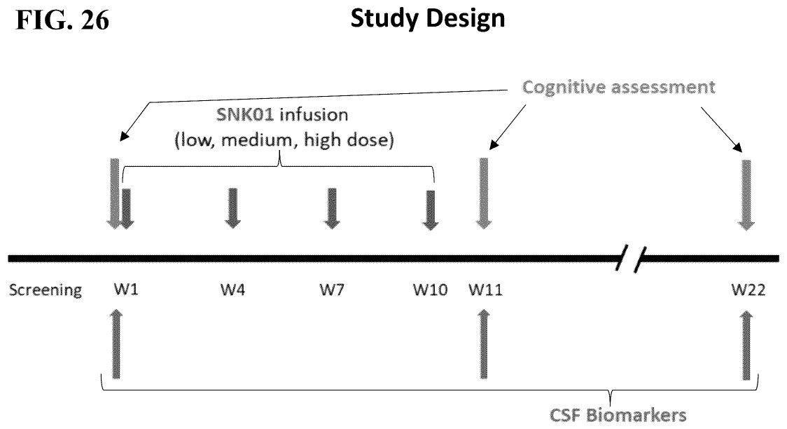

Cognitive assessments (CDR-SB, ADAS-Cog and MMSE) and CSF analyses (by electrochemiluminescent multiplexed immunoassays) were performed at baseline and at one week and 12 weeks after the final dose (Weeks 11 and 22, respectively) (See Study Design, FIG. 26).

FIG. 26 shows the study design for the SNK01 infusion assessment including screening, timing, and dosing of infusions, cognitive assessment and CSF biomarkers.

Ten subjects with mild (n=5) and moderate to severe AD (n=5) were enrolled in the three dose-escalation cohorts. Median age was 79 (56-85). Baseline median scores for CDR-SB, ADAS-Cog, and MMSE were 9 (4-18), 27.5 (18-65), and 14 (2-23), respectively. SNK01 was successfully activated/expanded from all enrolled subjects' peripheral blood and then administered. No treatment related adverse events have been observed to date.

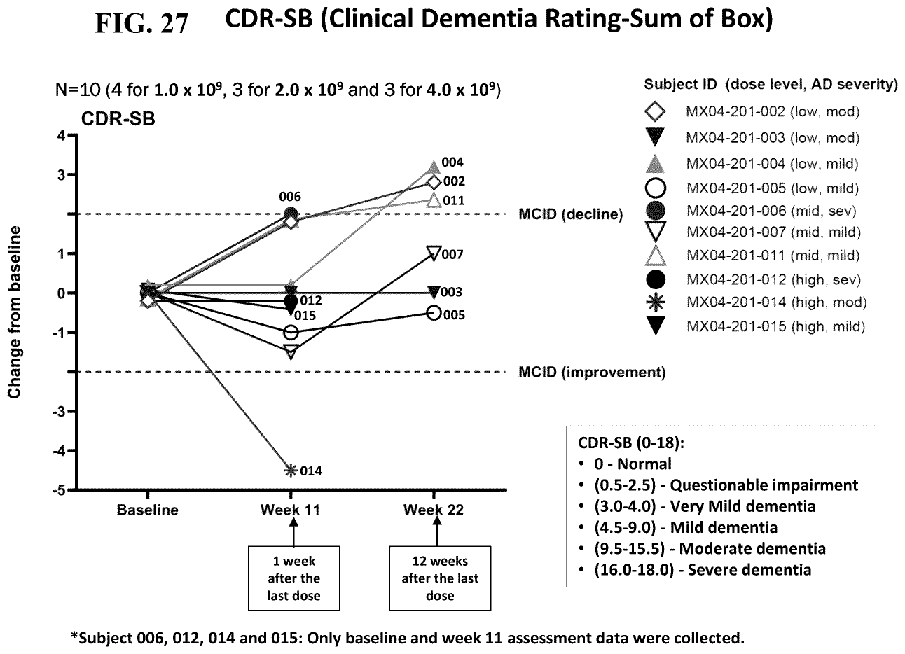

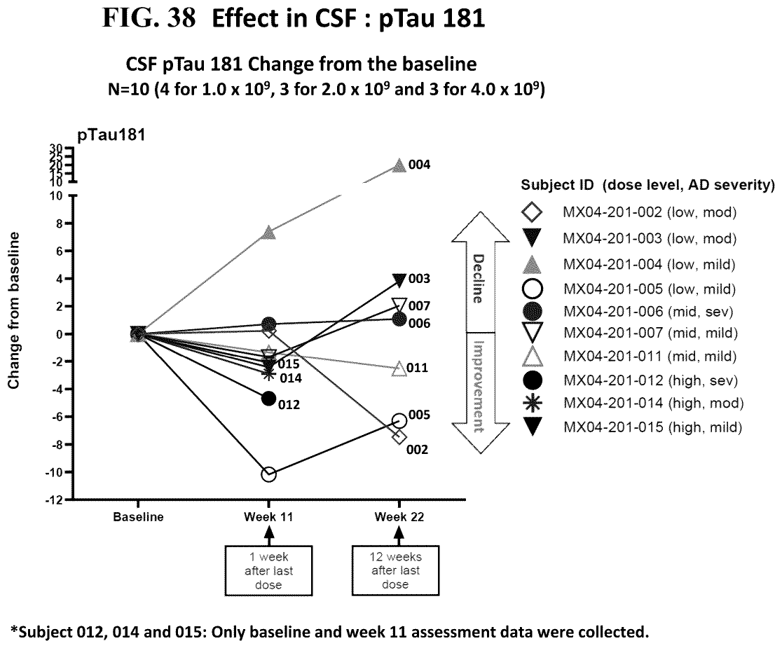

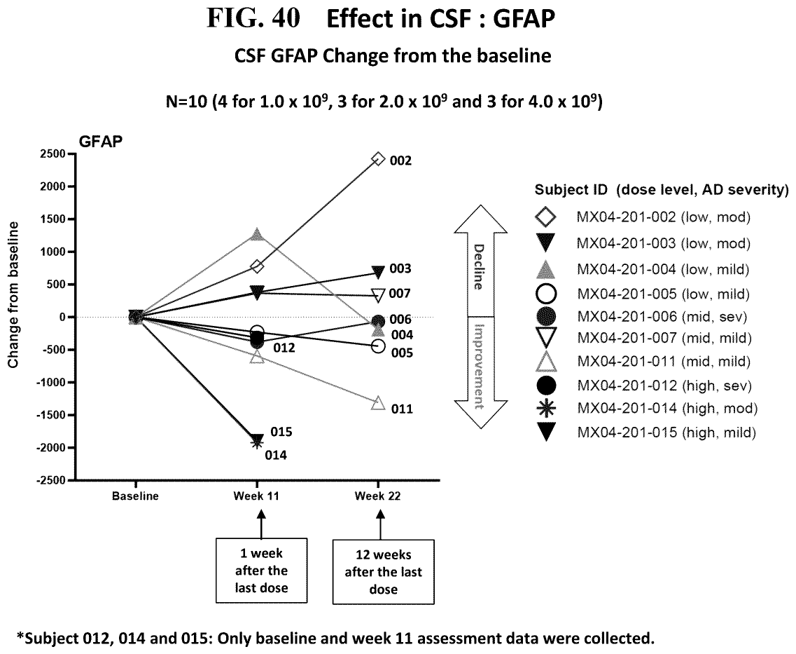

Treatment with SNK01 showed a tendency to stabilize or improve cognition by cognitive assessments with changes in some CSF biomarker levels when tested 1 week after the last dose (FIGS. 27-45). Some subjects maintained this treatment effect and biomarker levels when tested at 12 weeks after the last dose. Especially, subject 014 treated with high dose showed a marked improvement of cognition by cognitive assessments as well as favorable changes in GFAP and p-tau 181 levels (FIGS. 38, 40).

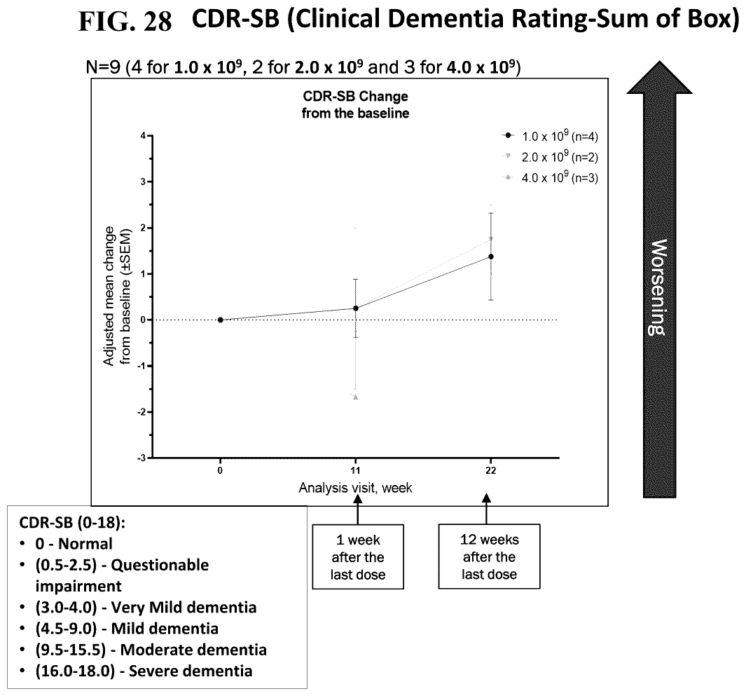

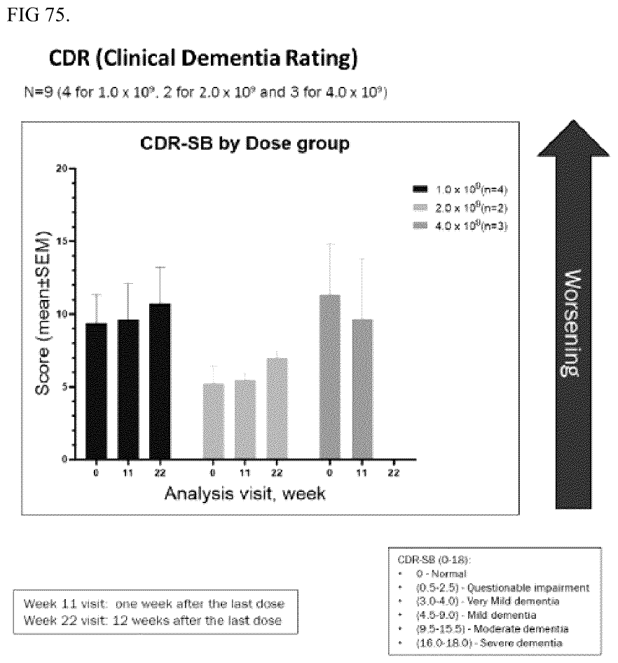

FIG. 27 shows a line graph depicting the Clinical Dementia Rating-Sum of Box (CDR-SB) scores of subjects treated with SNK01. FIG. 28 summarizes FIG. 27 and shows a line graph depicting the mean change from baseline in the Clinical Dementia Rating-Sum of Box (CDR-SB) scores of subjects treated with different doses of SNK01 grouped according to dosage. The underlying data includes the data plotted in FIG. 6B, which is presented using a Clinical Dementia Rating (CDR). FIG. 75 show a bar graph depicting the change in Clinical Dementia Rating (CDR) of subjects treated with different doses of NK cells. The underlying data includes the data plotted in FIG. 6A.

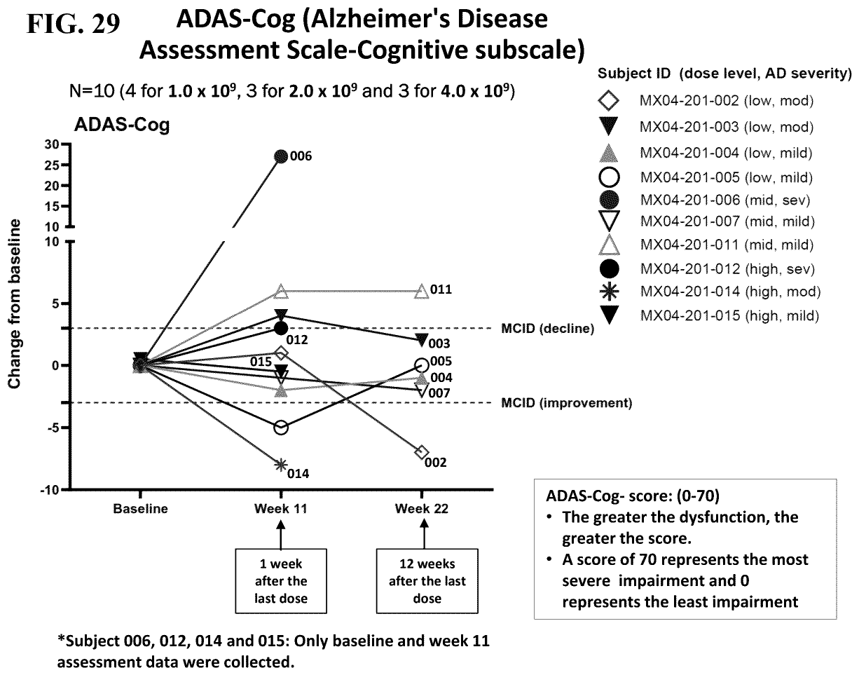

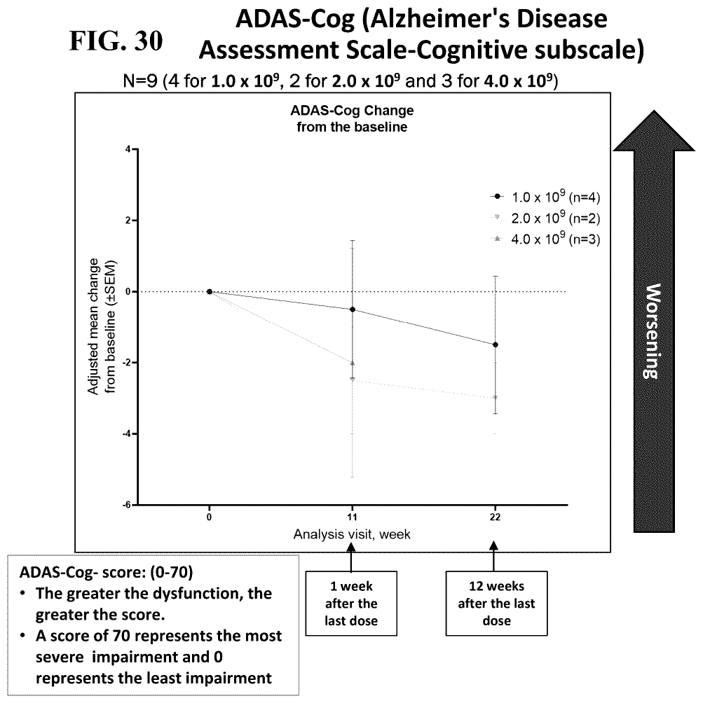

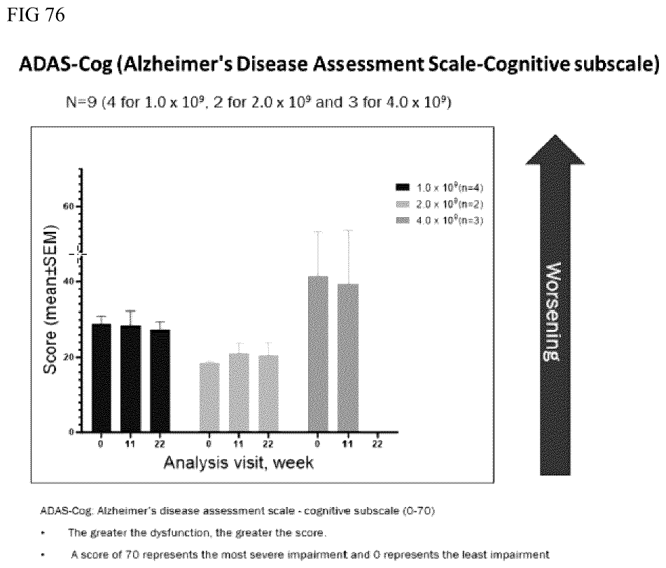

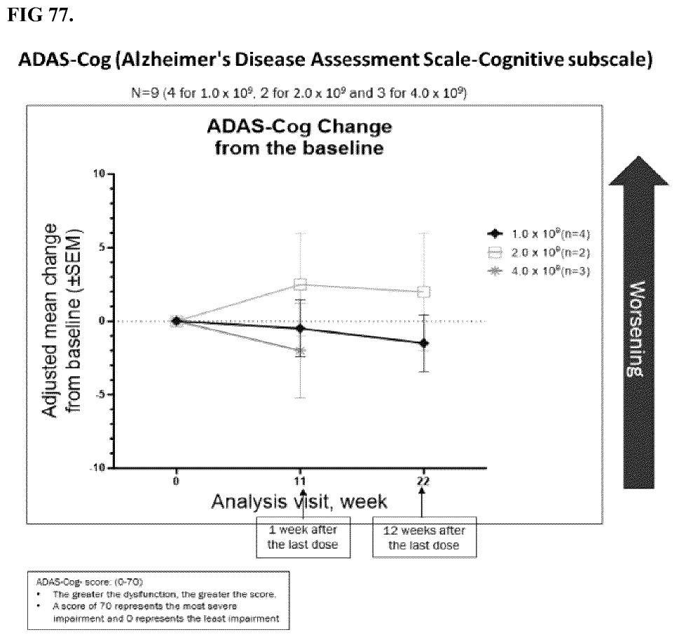

FIG. 29 shows a line graph depicting the change in Alzheimer's disease assessment scale-cognitive subscale (ADAS-Cog) scores (in a range of 0-70) of subjects treated with SNK01. FIG. 77 summarizes FIG. 29 and shows a line graph depicting the mean change from baseline for Alzheimer's Disease Assessment Scale-Cognitive subscale (ADAS-Cog) scores of subjects treated with different doses of SNK01 grouped according to dosage. The underlying data includes the data plotted in FIG. 7B, which is presented using an Alzheimer's Disease Assessment Scale-Cognitive subscale in a range of 0-80. FIG. 76 is a bar graph depicting the change in Alzheimer's disease assessment scale-cognitive subscale (ADAS-Cog) scores of subjects treated with different doses of NK cells. The underlying data includes the data plotted in FIG. 7A. FIG. 30 is another line graph depicting the change in Alzheimer's disease assessment scale-cognitive subscale (ADAS-Cog) scores of subjects treated with different doses of NK cells.

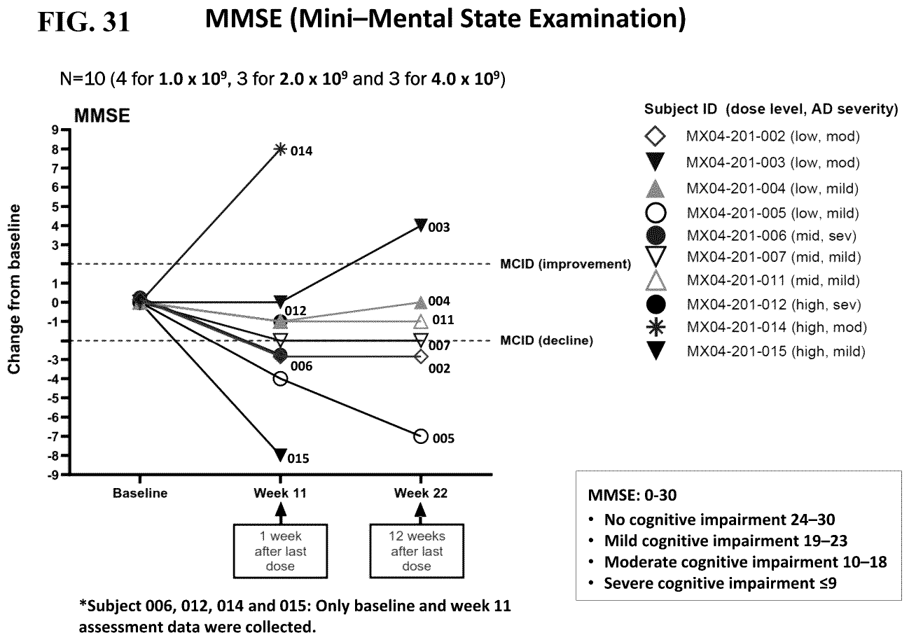

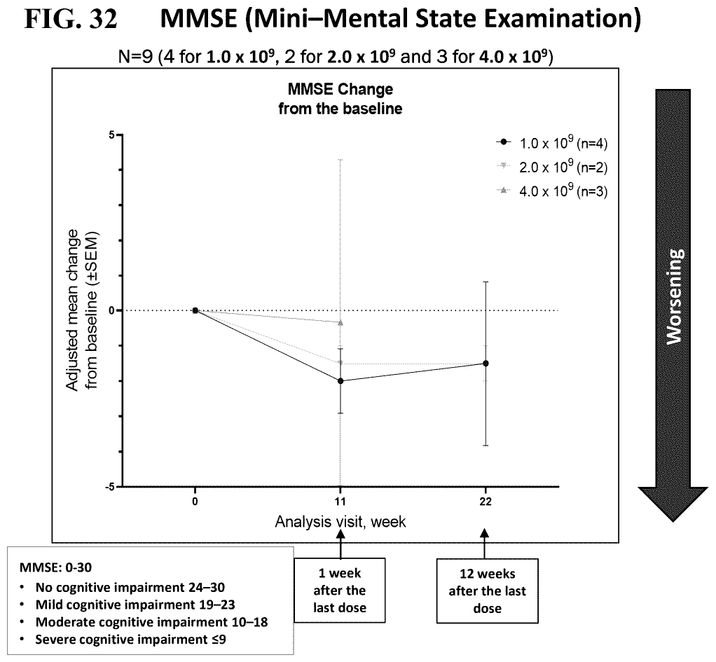

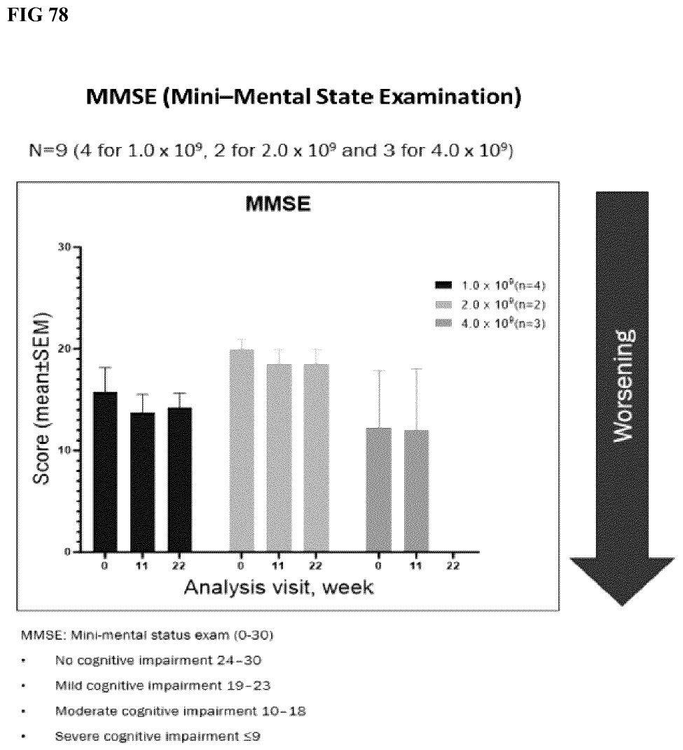

FIG. 31 shows a line graph depicting the Mini-Mental State Examination (MMSE) scores of subjects treated with SNK01. FIG. 32 summarizes FIG. 31 and shows a line graph depicting the mean change from baseline for the Mini-Mental State Examination (MMSE) scores of subjects treated with different doses of SNK01 grouped according to dosage. The underlying data includes the data plotted in FIG. 8B. FIG. 78 is a bar graph depicting the change in Mini-Mental State Examination (MMSE) scores of subjects treated with different doses of NK cells. The underlying data includes the data plotted in FIG. 8A.

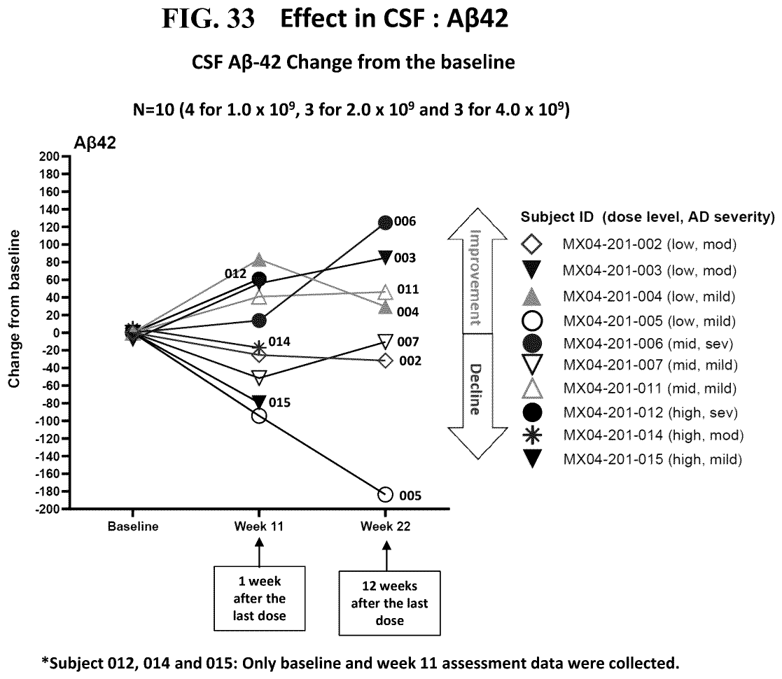

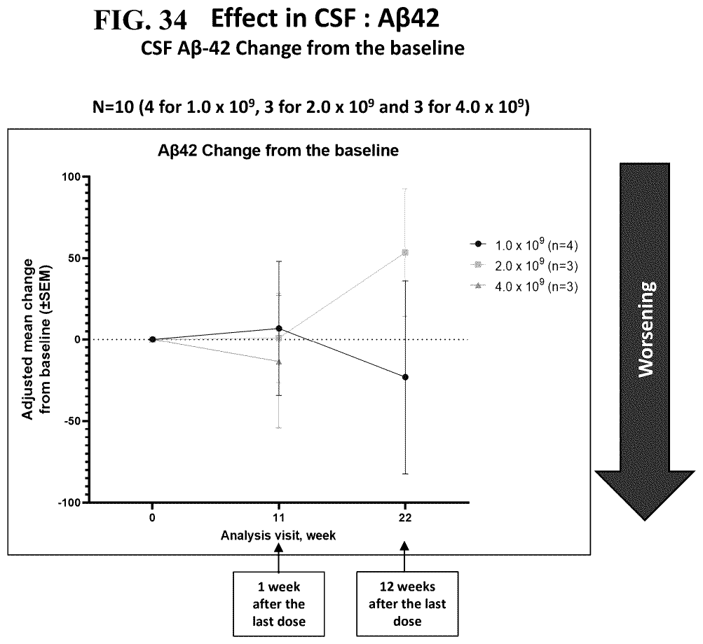

FIG. 33 shows a line graph depicting the change in Aβ-42 levels in the cerebrospinal fluid of subjects treated with different doses of SNK01. FIG. 34 summarizes FIG. 33 and shows a line graph depicting the mean change from baseline in Aβ-42 levels in the cerebrospinal fluid of subjects treated with different doses of SNK01 grouped according to dosage. The underlying data includes the data plotted in FIG. 9B.

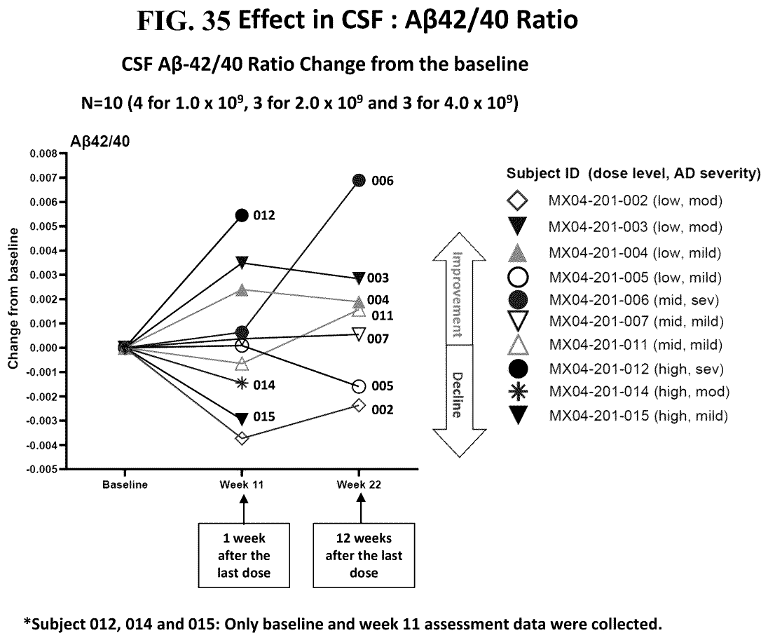

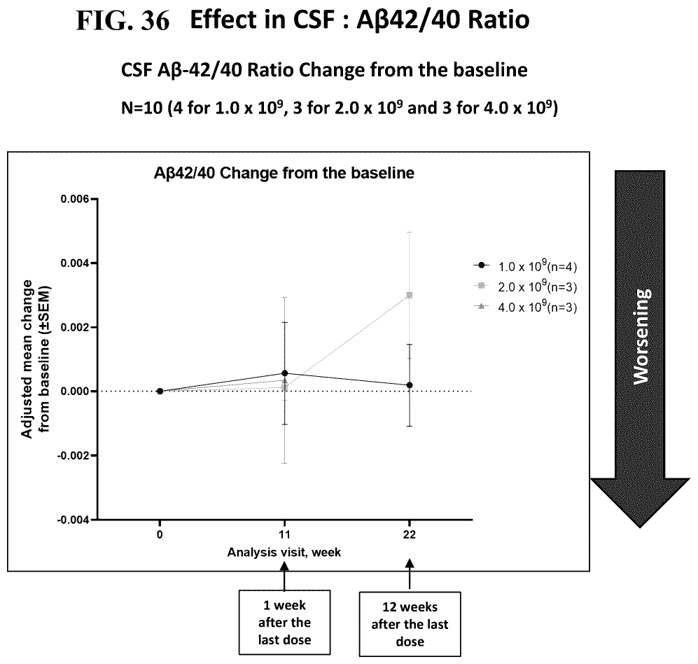

FIG. 35 shows a line graph depicting the change in Aβ-42/40 ratio in the cerebrospinal fluid of subjects treated with different doses of SNK01. FIG. 36 summarizes FIG. 35 and shows a line graph depicting the mean change from baseline in Aβ-42/40 ratio in the cerebrospinal fluid of subjects treated with different doses of SNK01 grouped according to dosage. The underlying data includes the data plotted in FIG. 11B. Decreased ratio of Aβ 42/40 is a strong marker of Alzheimer's disease and can be detected early in the disease progression, even before clinical dementia occurs.

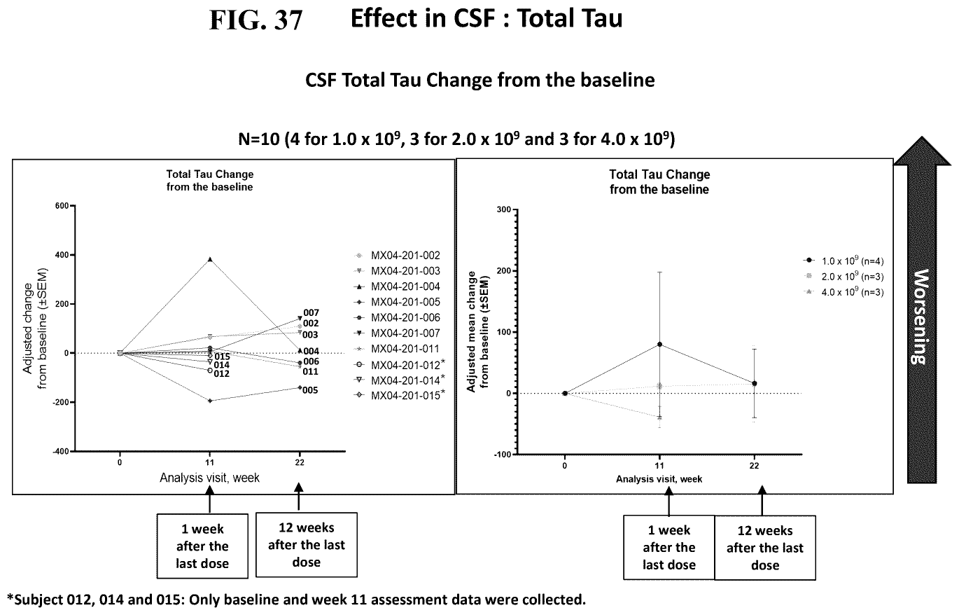

FIG. 37 shows line graphs depicting the change in total Tau levels in the cerebrospinal fluid of subjects treated with different doses of SNK01. Left panel shows changes in the subjects overtime. Right panel shows the mean change overtime, grouped according to dosage. CSF t-tau increase in AD patients may be caused by damaged neurons and the formation of tau tangles in the CNS in relation to neurodegeneration. Increases in total tau protein, as well as phosphorylated tau (p-tau), are also seen in CSF of AD patients. The underlying data includes the data plotted in FIG. 74.

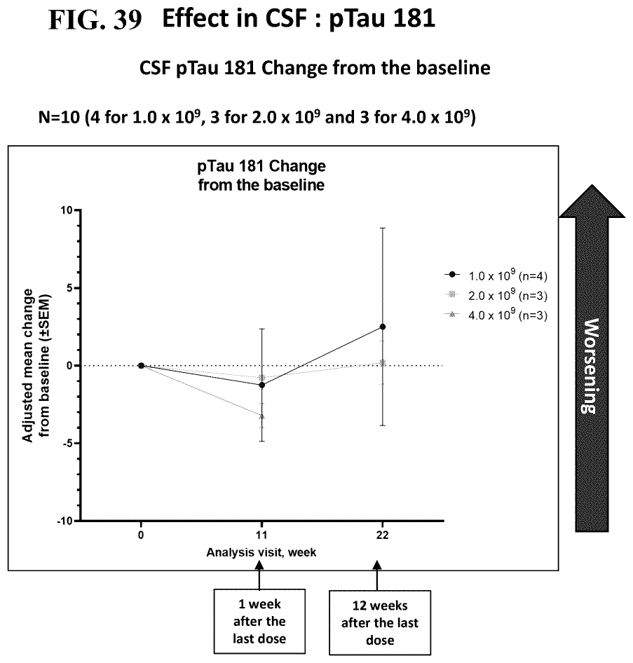

FIG. 38 shows a line graph depicting the change in p-tau 181 levels in the cerebrospinal fluid of subjects treated with different doses of SNK01. FIG. 39 summarizes FIG. 38 and shows a line graph depicting the mean change from baseline in p-tau 181 levels in the cerebrospinal fluid of subjects treated with different doses of SNK01 grouped according to dosage. The underlying data includes the data plotted in FIG. 12B.

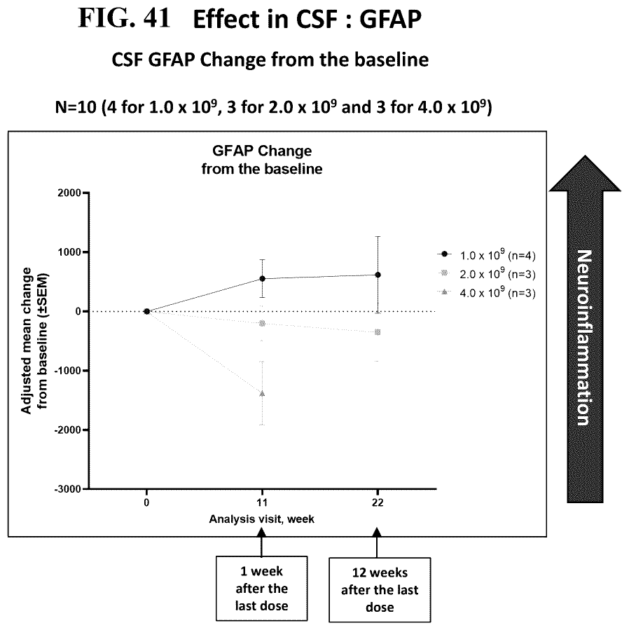

FIG. 40 shows a line graph depicting the change in GFAP levels in the cerebrospinal fluid of subjects treated with different doses of SNK01. FIG. 41 summarizes FIG. 40 and shows a line graph depicting the mean change from baseline in GFAP levels in the cerebrospinal fluid of subjects treated with different doses of SNK01 grouped according to dosage. The underlying data includes the data plotted in FIG. 13B. Glial fibrillary acidic protein (GFAP) is a marker of reactive astrogliosis that increases in the cerebrospinal fluid (CSF) and blood of individuals with Alzheimer disease (AD). GFAP correlates with astroglia activation. GFAP has been proposed as a biomarker of Alzheimer's disease (AD). GFAP expression correlates with AD plaque density. CSF concentration is elevated in AD.

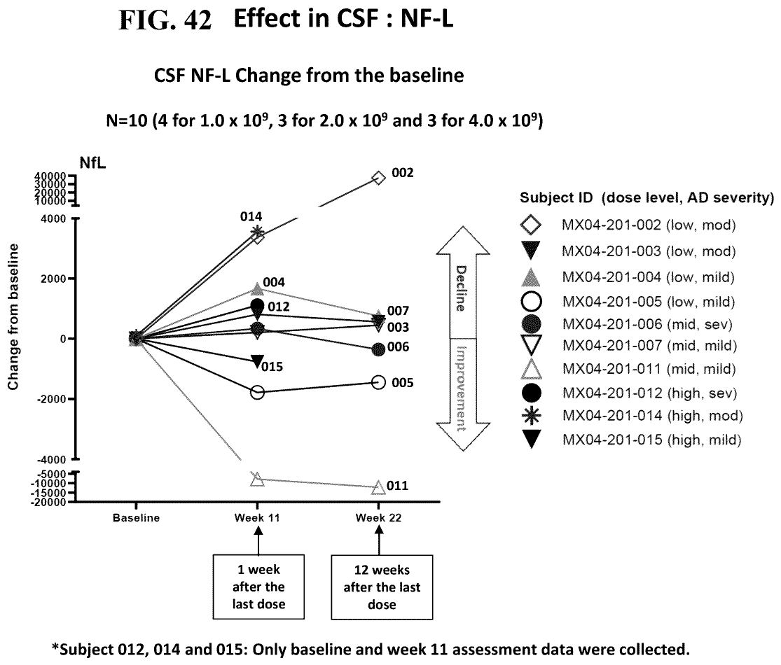

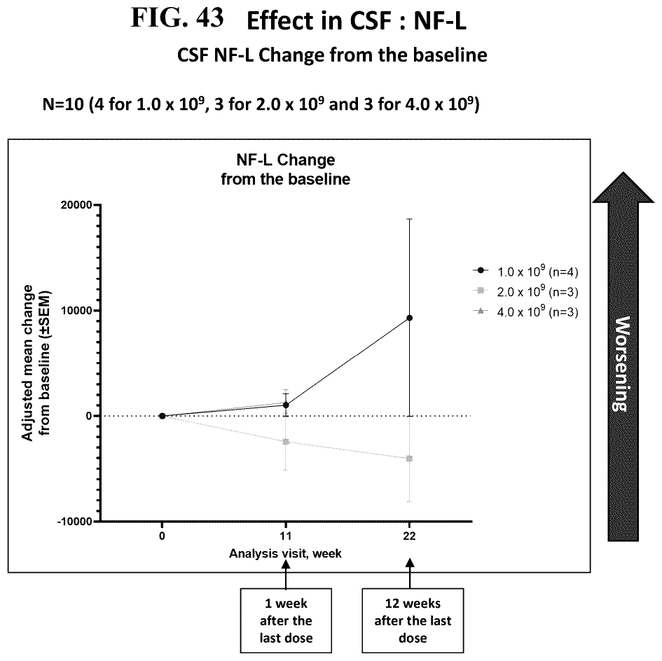

FIG. 42 shows a line graph depicting the change in NfL levels in the cerebrospinal fluid of subjects treated with different doses of SNK01. FIG. 43 summarizes FIG. 42 and shows a line graph depicting the mean change from baseline in NfL levels in the cerebrospinal fluid of subjects treated with different doses of SNK01 grouped according to dosage. The underlying data includes the data plotted in FIG. 14B. Cerebrospinal fluid (CSF) neurofilament light (NfL) is a biomarker of neurodegeneration in Alzheimer's disease (AD), the levels of which are significantly elevated in AD.

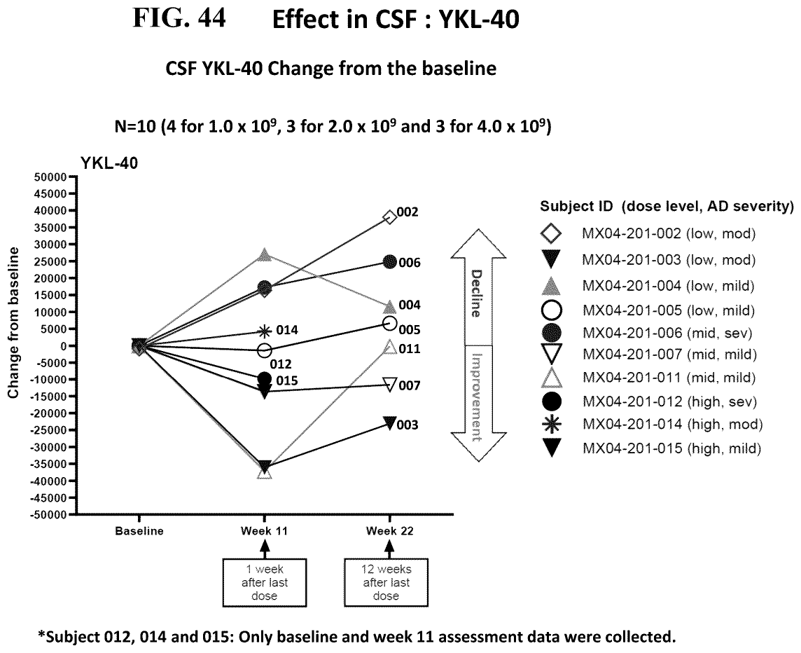

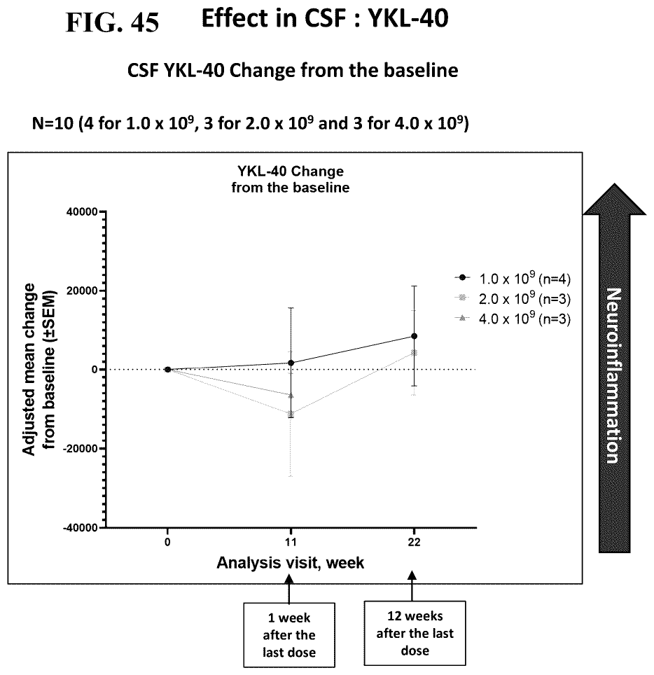

FIG. 44 shows a line graph depicting the change in YKL-40 levels in the cerebrospinal fluid of subjects treated with different doses of SNK01. FIG. 45 summarizes FIG. 44 and shows a line graph depicting the mean change from baseline in YKL-40 levels in the cerebrospinal fluid of subjects treated with different doses of NK cells grouped according to dosage. The underlying data includes the data plotted in FIG. 15B. YKL-40 (Chitinase 3-like I) is increased in CSF of Alzheimer's disease (AD) and frontotemporal lobar degeneration (FTLD) patients and is considered a potential neuroinflammatory biomarker.

Table 16 summarizes the results of this study. To select a minimal clinically important difference (MCID), published data across multiple years across multiple clinical assessments were analyzed. Each assessment scale was based on the MCID score change for mild AD, which was more conservative (as an example, a decline of 2 points in MMSE appears to be meaningful for mild AD only). The following MCIDs were used: CDR-SB: +/−2; MMSE: +/−2; ADAS-Cog: +/−3.

TABLE 16

| | Week 11 | Week 22 |
|---|---|---|
| Cognitive | (1 week post last dose) | Stable or Improved compared |
| Assessments | Stable or Improved | to Week 11 using MCID |
| CDR-SB | 7/10 (70%) | 4/6^{″} (67%) |
| ADAS-Cog | 6/10 (60%) | 5/6^{″} (83%) |
| MMSE | 5/10 (50%) | 5/6^{″} (83%) |

| | Stable or | | Rebound from Stable | |
|---|---|---|---|---|
| | Improved | Improved | or Improved | |
| Protein Biomarkers | | | | |
| Aβ42 | 5/10 (50%) | 5/10 (50%) | 1/4 | (25%)^{†} |
| Aβ42/40 | 6/10 (60%) | 3/10 (30%) | 3/5 | (60%)^{†} |
| pTau181 | 9/10 (90%) | 7/10 (70%) | 3/6 | (50%)^{† and} ** |
| Neuroinflammation | | | | |
| Markers | | | | |
| GFAP | 6/10 (60%) | 6/10 (60%) | 1/3 | (33%)^{† and} ** |
| NfL | 5/10 (50%) | 3/10 (30%) | 1/4 | (25%) ^{♦} |
| YKL-40 | 6/10 (60%) | 5/10 (50%) | 4/4 | (100%)^{† and ♦} |

^{″}2 subjects early terminated before reaching Week 22, 2 subjects are pending Week 22 visits.
**2 subjects are pending Week 22 visits.
^{†}1 subject early terminated from the study
^{♦} 1 subject is pending Week 22 visit.

Conclusions: SNK01 appeared to be safe and well tolerated. SNK01 showed clinical activity in AD, with a dose response seen in CDR-SB and MMSE assessment scores. In addition, based on the CSF biomarker data, SNK01 given via peripheral IV seems to reduce p-tau181 and neuroinflammation in a dose dependent manner by crossing the blood brain barrier. Finally, there appears to be a rebound effect in these biomarkers when SNK01 treatment is discontinued.

### Example 12 Alzheimer's Disease Markers in Plasma

Objective and Method: Alzheimer's disease (AD) is a dual proteinopathy characterized by extracellular deposits of fibrillar amyloid-beta peptides and aggregates of the phosphorylated microtubule-associated protein tau in neurofibrillary tangles.

In the literature, sensitivity and specificity may be observed for AD neuropathological change in plasma biomarkers related to amyloid, tau, and neurodegeneration. Blood biomarkers indicative of AD pathology are altered in both preclinical and symptomatic stages of the disease. Distinctive biomarkers may be suitable for the identification of AD pathology or monitoring of disease progression. Blood biomarkers that correlate with changes in cognition and atrophy during the course of the disease are used in clinical trials to identify successful interventions and thereby accelerate the development of efficient therapies. Lower plasma Aβ42/Aβ40 ratio and higher phosphorylated tau (p-tau181), Glial fibrillary acidic protein (GFAP), and Neurofilament light (NfL) are associated with cognitive decline and increased AD-PET load. (Smimov et al. 2022, Ashton et al. 2022, Chatterjee et al. 2023)

Plasma from subjects with AD who participated in the autologous NK cell therapy (SNK01) study were collected and used to access the cell therapy treatment responses. Blood was drawn from a forearm vein into EDTA citrate vacutainer tubes and centrifuged at 1000×g for 10 min at 4° C. in a tabletop centrifuge within 1 h or less of blood draw. Plasma was separated and aliquoted into polypropylene cryotubes, snap frozen and stored at −80° until biomarkers analyses were conducted.

Quantification of the presence of the AD biomarkers and proteins in plasma were conducted to evaluate the effect of three levels of dosage of SNK01 treatments for mild, moderate and severe AD subjects. The biomarkers panel included Amyloid Beta 42 (Aβ42), Aβ42/Aβ40 ratio, p-tau 181, NfL, GFAP, Chitinase-3-like protein 1 (YKL-40), Interleukin 6 (IL-6) and Tumor necrosis factor α (TNF-α).

The quantification of the markers was conducted using Meso Scale Discovery (MSD) multiplexed sandwich immunoassays. MSD assays are designed to measure levels of peptide and protein in biological samples. The multiplexed assays use electrochemiluminescent labels that are conjugated to detect antibodies. The labels allow for ultra-sensitive detection. Analytes in the sample bind to capture antibodies immobilized on the working electrode surface and recruitment of the detection antibodies conjugated with electrochemiluminescent labels. Electricity is applied to the electrodes by an MSD instrument leading to light emission by the conjugated labels. Light intensity is then measured to quantify analytes in the sample.

Background of the markers: Plasma Aβ42/Aβ40 ratio is a diagnostic biomarker of AD during both predementia and dementia stages with comparable correlation to level of CSF Aβ42/Aβ40 ratio. The ratios reflect AD-type pathology better, whereas decline in Aβ42 is also associated with non-AD subcortical pathologies. Studies suggested that the ratios rather than Aβ42 can be used in the clinical work-up of AD. (Janelidze et al. 2016, Wilczynska el at. 2021)

The tangles characteristic of AD are made up of filaments formed from an abnormally phosphorylated form of tau called phospho-tau (p-tau). P-tau is believed to reflect neurofibrillary pathology. Level of plasma p-tau 181 correlates to CSF p-tau 181, tau PET and cognitive impairment (Janelidze et al. 2016, Tatebe et al. 2017, Mielke et al. 2018, Yang et al. 2018)

NfL, an intermediate filament protein expressed exclusively in neurons, has emerged as a promising blood-based biomarker of neurodegeneration in several neurological disorders, including AD. In sporadic AD, the concentrations of NfL in CSF and blood are significantly increased in both the prodromal and dementia stages of the disease, in which they associate with cognitive decline and disease-related structural brain changes. Consistent observations found that the concentration of NfL in plasma correlates positively with those in CSF, suggesting that NfL in blood is likely to originate from the central nervous system (CNS). In addition, NfL in both CSF and plasma were higher in patients with mild cognitive impairment (MCI) and AD dementia compared to cognitive unimpaired subjects suggesting that NfL tracks neurodegeneration. (Khalil et al., 2018, Mattsson et al., 2017, 2019; Olsson et al., 2019; Zetterberg et al., 2016)

GFAP is an intermediate filament structural protein involved in cytoskeleton assembly and integrity, expressed in high abundance in activated glial cells. Neuronal stress, caused by either disease or injury, evokes astrocyte activation as a response, including hypertrophy, proliferation, and increased GFAP expression. GFAP is a marker of reactive astrogliosis that increases in CSF and blood of individuals with Alzheimer disease (AD) (Ganne, Akshatha et al. 2022)

YKL-40 is an inflammatory marker considered as a potential biomarker of dementia, neoplastic diseases, and chronic inflammation. It is elevated in the brain, CSF and in serum in several neurological and neurodegenerative diseases associated with inflammatory processes. YKL-40 is a highly sensitive and specific marker that differentiates healthy individuals from patients with Alzheimer's, vascular or mixed dementia. Studies shown that the increase in peripheral blood YKL-40 concentration in AD results from the activation of proinflammatory cells due to cell death caused by the accumulation of beta amyloid. YKL-40 correlated with the concentrations of other markers (t-tau and Aβ42/Aβ40), the severity of dementia as reflected by negative correlation with the MMSE score, and the parameters of inflammation (Llorens et al. Molecular Neurodegeneration 2017, Wilczynska et al. 2021)

Interleukin 6 (IL-6) is upregulated in AD brain and plasma, correlates positively with brain inflammation and inversely with MMSE scores. IL-6 is a component of early-stage amyloid plaque formation in AD brains and has been implicated in tau phosphorylation, synapse loss, and learning deficits in mice. IL-6 is increased in both CSF and plasma of mild cognitive impairment (MCI) and AD patients compared to healthy individuals. (Silva et al. 2021)

Tumor necrosis factor α (TNF-α) plays an essential role in the cytokine cascade during neuroinflammation response. The levels of TNF-α are significantly elevated in blood and CNS of patients with AD. The role of TNF-α in AD pathology was further suggested by studies in which significant elevation of TNF-α levels in the CSF and serum of patients with AD correlated with disease progression. (Chang et al. 2017)

### REFERENCES

Ganne, Akshatha et al. “Glial Fibrillary Acidic Protein: A Biomarker and Drug Target for Alzheimer's Disease.” Pharmaceutics vol. 14, 7 1354. 26 Jun. 2022 doi:10.3390/pharmaceutics14071354
Wilczyńska K, Maciejczyk M, Zalewska A, Waszkiewicz N. Serum Amyloid Biomarkers, Tau Protein and YKL-40 Utility in Detection, Differential Diagnosing, and Monitoring of Dementia. Front Psychiatry. 2021 Sep. 13; 12:725511. doi: 10.3389/fpsyt.2021.725511. PMID: 34589009; PMCID: PMC8473887.
Ashton, N.J., Janelidze, S., Mattsson-Carlgren, N. et al. Differential roles of Aβ42/40, p-tau231 and p-tau217 for Alzheimer's trial selection and disease monitoring. Nat Med 28, 2555-2562 (2022).
Smimov D S, Ashton N J, Blennow K, Zetterberg H, Simrén J, Lantero-Rodriguez J, Karikari T K, Hiniker A, Rissman R A, Salmon D P, Galasko D. Plasma biomarkers for Alzheimer's Disease in relation to neuropathology and cognitive change. Acta Neuropathol. 2022 April; 143(4):487-503. doi: 10.1007/s00401-022-02408-5. Epub 2022 Feb. 23. PMID: 35195758; PMCID: PMC8960664.
Janelidze S, Mattsson N, Palmqvist S, et al. Plasma P-tau181 in Alzheimer's disease: relationship to other biomarkers, differential diagnosis, neuropathology and longitudinal progression to Alzheimer's dementia. Nature Medicine. 2020 March; 26(3):379-386. DOI: 10.1038/s41591-020-0755-1. PMID: 32123385.
Llorens, F., Thiine, K., Tahir, W. et al. YKL-40 in the brain and cerebrospinal fluid of neurodegenerative dementias. Mol Neurodegeneration 12, 83 (2017).
Xinyu Li, Weiren Wang; Levels of IL-6 in peripheral blood and cerebrospinal fluid of Alzheimer's disease: a meta-analysis. International Journal of Frontiers in Medicine. ISSN 2706-6819 Vol. 5, Issue 2: 53-60, DOI: 10.25236/IJFM.2023.050210
Chatterjee P, Pedrini S, Doecke J D, Thota R, Villemagne V L, Doré V, Singh A K, Wang P, Rainey-Smith S, Fowler C, Taddei K, Sohrabi H R, Molloy M P, Ames D, Maruff P, Rowe C C, Masters C L, Martins R N; AIBL Research Group. Plasma Aβ42/40 ratio, p-tau181, GFAP, and NfL across the Alzheimer's disease continuum: A cross-sectional and longitudinal study in the AIBL cohort. Alzheimers Dement. 2023 April; 19(4):1117-1134. doi: 10.1002/alz.12724. Epub 2022 Jul. 21. PMID: 36574591.
Tatebe, H., T. Kasai, T. Ohmichi, Y. Kishi, T. Kakeya, M. Waragai, M. Kondo, D. Allsop and T. Tokuda (2017). “Quantification of plasma phosphorylated tau to use as a biomarker for brain Alzheimer pathology: pilot case-control studies including patients with Alzheimer's disease and down syndrome.” Mol Neurodegener 12(1): 63.
Mielke, M. M., C. E. Hagen, J. Xu, X. Chai, P. Vemuri, V. J. Lowe, D. C. Airey, D. S. Knopman, R. O. Roberts, M. M. Machulda, C. R. Jack, Jr., R. C. Petersen and J. L. Dage (2018). “Plasma phospho-tau181 increases with Alzheimer's disease clinical severity and is associated with tau- and amyloid-positron emission tomography.” Alzheimers Dement 14(8): 989-997.
Yang, C. C., M. J. Chiu, T. F. Chen, H. L. Chang, B. H. Liu and S. Y. Yang (2018). “Assay of Plasma Phosphorylated Tau Protein (Threonine 181) and Total Tau Protein in Early-Stage Alzheimer's Disease.” J Alzheimers Dis 61(4): 1323-1332.
Stocker H, Beyer L, Perna L, Rujescu D, Holleczek B, Beyreuther K, Stockmann J, Schottker B, Gerwert K, Brenner H. Association of plasma biomarkers, p-tau181, glial fibrillary acidic protein, and neurofilament light, with intermediate and long-term clinical Alzheimer's disease risk: Results from a prospective cohort followed over 17 years. Alzheimers Dement. 2023 January; 19(1):25-35. doi: 10.1002/alz.12614. Epub 2022 Mar. 2. PMID: 35234335.
Andersson E, Janelidze S, Lampinen B, Nilsson M, Leuzy A, Stomrud E, Blennow K, Zetterberg H, Hansson O. Blood and cerebrospinal fluid neurofilament light differentially detect neurodegeneration in early Alzheimer's disease. Neurobiol Aging. 2020 November; 95:143-153. doi: 10.1016/j.neurobiolaging.2020.07.018. Epub 2020 Jul. 25. PMID: 32810755; PMCID: PMC7649343.
Khalil M., Teunissen C. E., Otto M., Piehl F., Sormani M. P., Gattringer T., Barro C., Kappos L., Comabella M., Fazekas F., Petzold A., Blennow K., Zetterberg H., Kuhle J. Neurofilaments as biomarkers in neurological disorders. Nat. Rev. Neurol. 2018; 14:577-589. [PubMed: 30171200]
Zetterberg H., Skillbäck T., Mattsson N., Trojanowski J. Q., Portelius E., Shaw L. M. Association of cerebrospinal fluid neurofilament light concentration with Alzheimer disease progression. JAMA Neurol. 2016; 73:60-67. [PMCID: PMC5624219][PubMed: 26524180]
Stocker H, Beyer L, Perna L, Rujescu D, Holleczek B, Beyreuther K, Stockmann J, Schottker B, Gerwert K, Brenner H. Association of plasma biomarkers, p-tau181, glial fibrillary acidic protein, and neurofilament light, with intermediate and long-term clinical Alzheimer's disease risk: Results from a prospective cohort followed over 17 years. Alzheimers Dement. 2023 January; 19(1):25-35. doi: 10.1002/alz.12614. Epub 2022 Mar. 2. PMID: 35234335.
Lyra e Silva, N. M., Gonçalves, R. A., Pascoal, T. A. et al. Pro-inflammatory interleukin-6 signaling links cognitive impairments and peripheral metabolic alterations in Alzheimer's disease. Transl Psychiatry 11, 251 (2021).
Chang, R., Yee, K. L., & Sumbria, R. K. (2017). Tumor necrosis factor α Inhibition for Alzheimer's Disease. Journal of central nervous system disease, 9, 1179573517709278.

### Example 13. Single Center, Open-Label, Phase 1 Study to Evaluate the Safety, Tolerability, and Exploratory Efficacy of SNK01 in Subjects with Mild Cognitive Impairment (MCI) and Alzheimer's Disease (AD) (Study SNK01-MX04)

This non-limiting example shows the results of a phase 1 study to evaluate the safety, tolerability, and exploratory efficacy of SNK01 in subjects with mild cognitive impairment (MCI) and Alzheimer's Disease (AD), as described in Examples 10 and 11. This example includes the subjects and data described in Example 10, and further adds additional subjects and corresponding data, as described in Example 11. Measurement of marker levels were carried out as described in Examples 10-12.

Table 17 summarizes some of the results of this example and shows the percentage of patients with positive outcomes in their plasma samples. It shows the number of patients with a “stable or improved” outcome over the total number of patients.

TABLE 17

PLASMA

| | |
|---|---|
| W 11 | W 22 |

Stable or Improved

| | | |
|---|---|---|
| Protein Biomarkers | | |
| Aβ42 | 5/10 = 50% | 5/7 = 71% |
| Aβ42/40 | 3/10 = 30% | 4/7 = 57% |
| p-tau181 | 8/10 = 80% | 7/7 = 100% |
| Neuroinflammation | | |
| Markers | | |
| GFAP | 5/10 = 50% | 4/7 = 57% |
| NfL | 7/10 = 70% | 4/7 = 57% |
| YKL-40 | 9/10 = 90% | 5/7 = 71% |

Table 18 shows the Clinical Dementia Rating-Sum of Box (CDR-SB). CDR-SB of subjects in this study at different time points. The CDR sum of box score scale indicates the following: 0-Normal, (0.5-2.5)—Questionable impairment, (3.0-4.0)—Very Mild dementia, (4.5-9.0)—Mild dementia, (9.5-15.5)—Moderate dementia, (16.0-18.0)—Severe dementia. The data is also plotted in FIG. 27 as the change from baseline.

TABLE 18
CDR-SB (Clinical Dementia Rating-Sum of Box)

| Analysis | | | | | | | | | | |
|---|---|---|---|---|---|---|---|---|---|---|
| Visit, | MX04- | MX04- | MX04- | MX04- | MX04- | MX04- | MX04- | MX04- | MX04- | MX04- |
| week | 201-002 | 201-003 | 201-004 | 201-005 | 201-006 | 201-007 | 201-011 | 201-012 | 201-014 | 201-015 |
| 0 | 13 | 12 | 8 | 4.5 | 16 | 6.5 | 4 | 18 | 10 | 6 |
| 11 | 15 | 12 | 8 | 3.5 | 18 | 5 | 6 | 18 | 5.5 | 5.5 |
| 22 | 16 | 12 | 11 | 4 | | 7.5 | 6.5 | | | |

Table 19 shows the Mini-Mental State Examination (MMSE). MMSE (0-30): no cognitive impairment 24-30; mild cognitive impairment 19-23; moderate cognitive. The data is also plotted in FIG. 31 as the change from baseline.

TABLE 19
MMSE (Mini-Mental State Examination)

| Analysis | | | | | | | | | | |
|---|---|---|---|---|---|---|---|---|---|---|
| Visit, | MX04- | MX04- | MX04- | MX04- | MX04- | MX04- | MX04- | MX04- | MX04- | MX04- |
| week | 201-002 | 201-003 | 201-004 | 201-005 | 201-006 | 201-007 | 201-011 | 201-012 | 201-014 | 201-015 |
| 0 | 14 | 13 | 13 | 23 | 14 | 19 | 21 | 2 | 14 | 21 |
| 11 | 11 | 13 | 12 | 19 | 11 | 17 | 20 | 1 | 22 | 13 |
| 22 | 11 | 17 | 13 | 16 | | 17 | 20 | | | |

Table 20 shows the Alzheimer's Disease Assessment Scale-Cognitive subscale. ADAS (0-70): The greater the dysfunction, the greater the score. A score of 70 represents the most severe impairment and 0 represents the least impairment. The data is also plotted in FIG. 29 as the change from baseline.

TABLE 20
ADAS-Cog (Alzheimer's Disease Assessment Scale-Cognitive subscale)

| Analysis | | | | | | | | | | |
|---|---|---|---|---|---|---|---|---|---|---|
| Visit, | MX04- | MX04- | MX04- | MX04- | MX04- | MX04- | MX04- | MX04- | MX04- | MX04- |
| week | 201-002 | 201-003 | 201-004 | 201-005 | 201-006 | 201-007 | 201-011 | 201-012 | 201-014 | 201-015 |
| 0 | 33 | 31 | 28 | 24 | 27 | 19 | 18 | 65 | 32 | 27 |
| 11 | 34 | 35 | 26 | 19 | 54 | 18 | 24 | 68 | 24 | 26 |
| 22 | 26 | 33 | 27 | 24 | | 17 | 24 | | | |

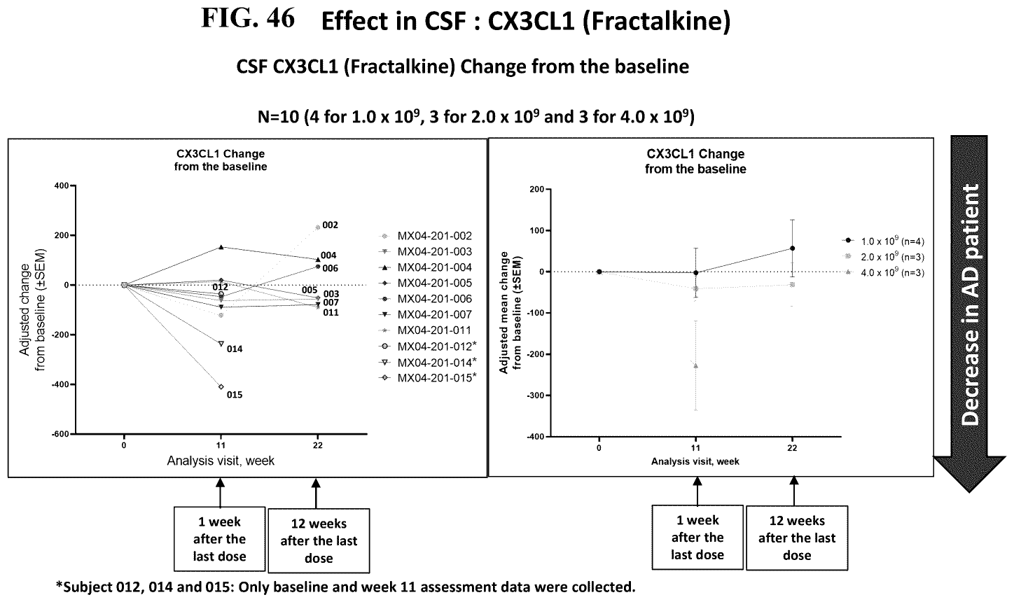

FIG. 46 shows line graph depicting the change in baseline CX3CL1 (Fractalkine) levels in the cerebrospinal fluid of subjects treated with different doses of SNK01. Left panel shows changes in the subjects over time. Right panel shows the mean change over time, grouped according to dosage. The underlying data includes the data plotted in FIG. 16B.

Decreased CX3CL1 concentrations are found in the CSF of AD patients compared to non-AD patients. CX3C chemokine ligand 1 (CX3CL1, also named fractalkine) plays an important role in reducing neuroinflammation and is highly expressed in the main area of pathological changes in AD, such as the hippocampus and cerebral cortex, and the expression level of CX3CL1 reflects the progression of the disease. The activation of microglial CX3CR1, the sole receptor for CX3CL1, reduces the activation of microglia, which contribute to the neuronal damage characteristic of AD. Therefore, alterations of CX3CR1 production in microglia can translate into the enhancement or inhibition of CX3CL1 anti-inflammatory effect.

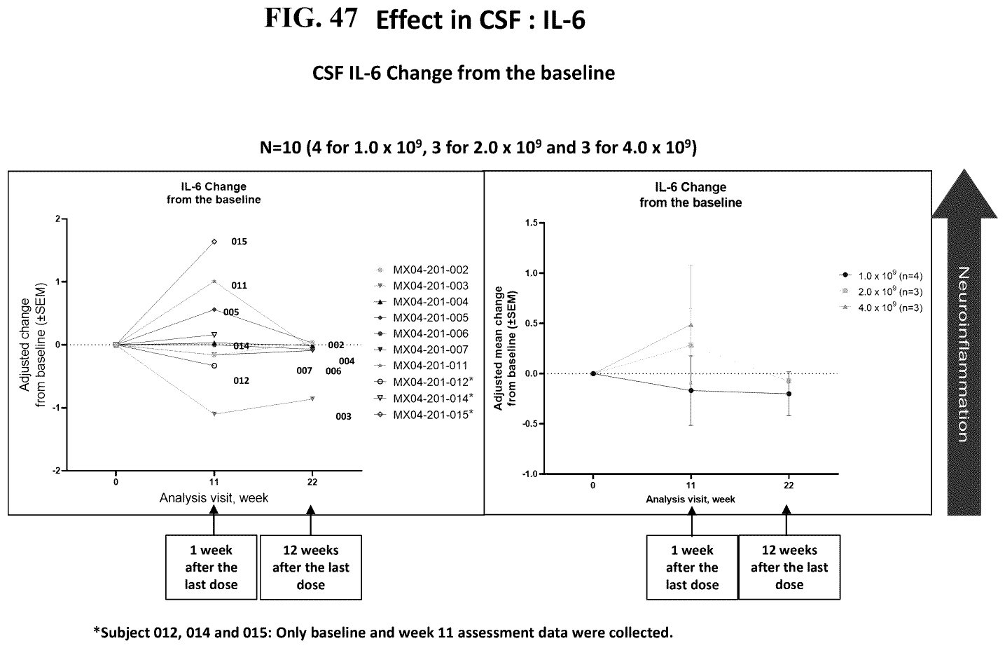

FIG. 47 shows line graphs depicting the change in baseline IL-6 levels in the cerebrospinal fluid of subjects treated with different doses of SNK01. Left panel shows changes in the subjects over time. Right panel shows the mean change over time, grouped according to dosage. The underlying data includes the data plotted in FIG. 17B.

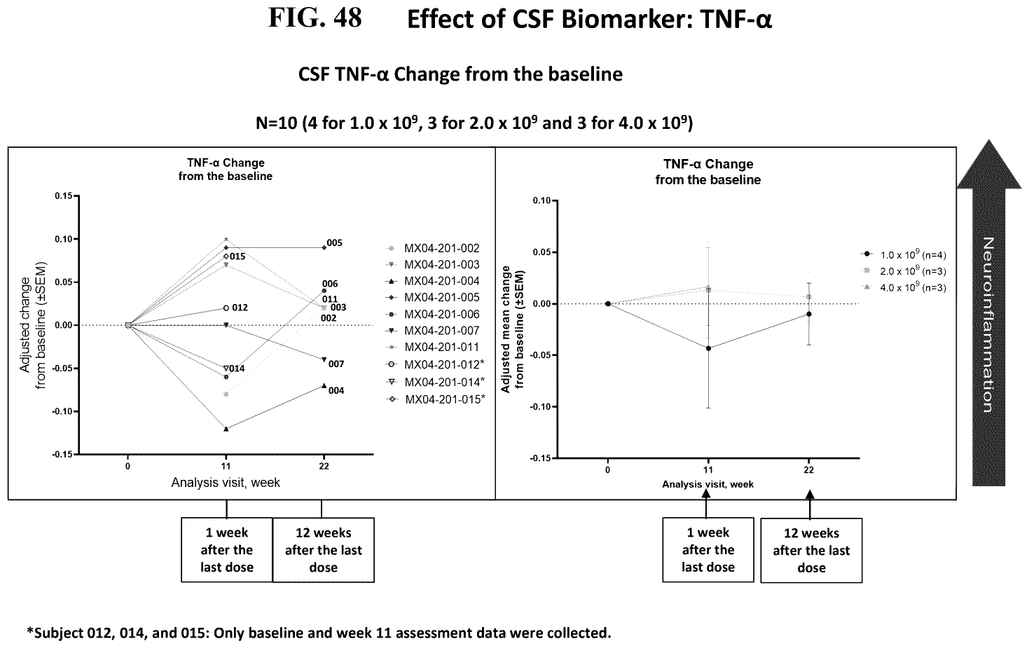

FIG. 48 shows line graphs depicting the change in baseline TNF-α levels in the cerebrospinal fluid of subjects treated with different doses of SNK01. Left panel shows changes in the subjects over time. Right panel shows the mean change over time, grouped according to dosage. The underlying data includes the data plotted in FIG. 18B.

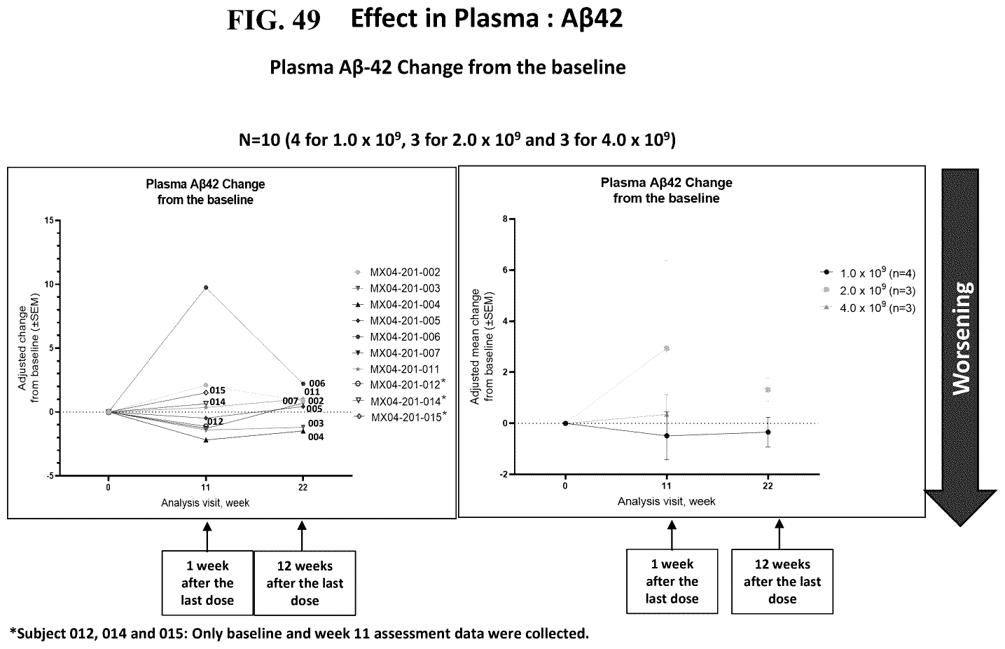

FIG. 49 shows line graphs of Aβ-42 changes in the plasma of subjects treated with SNK01. Right panel shows the mean change over time, grouped according to dosage.

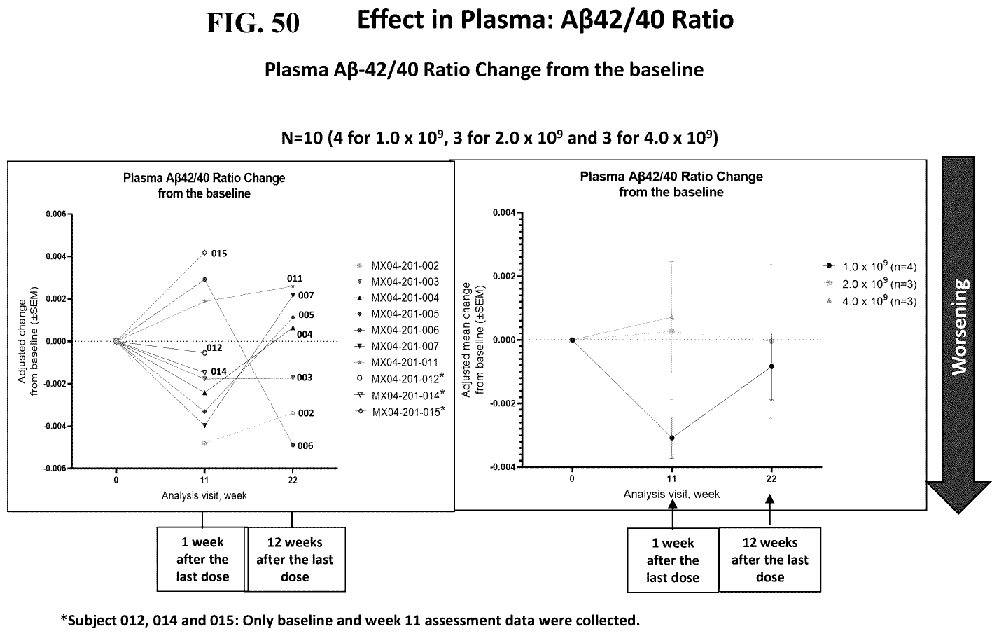

FIG. 50 shows line graphs of Aβ-42/40 ratio changes in the plasma of subjects treated with SNK01. Right panel shows the mean change over time, grouped according to dosage.

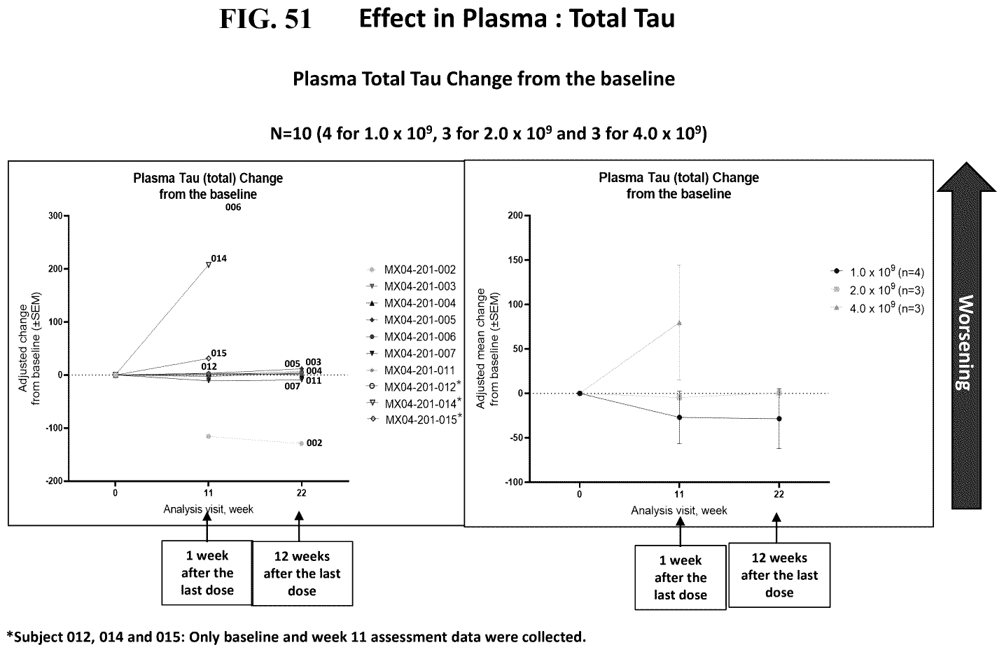

FIG. 51 shows line graphs of changes in total tau in the plasma of subjects treated with SNK01. Right panel shows the mean change over time, grouped according to dosage.

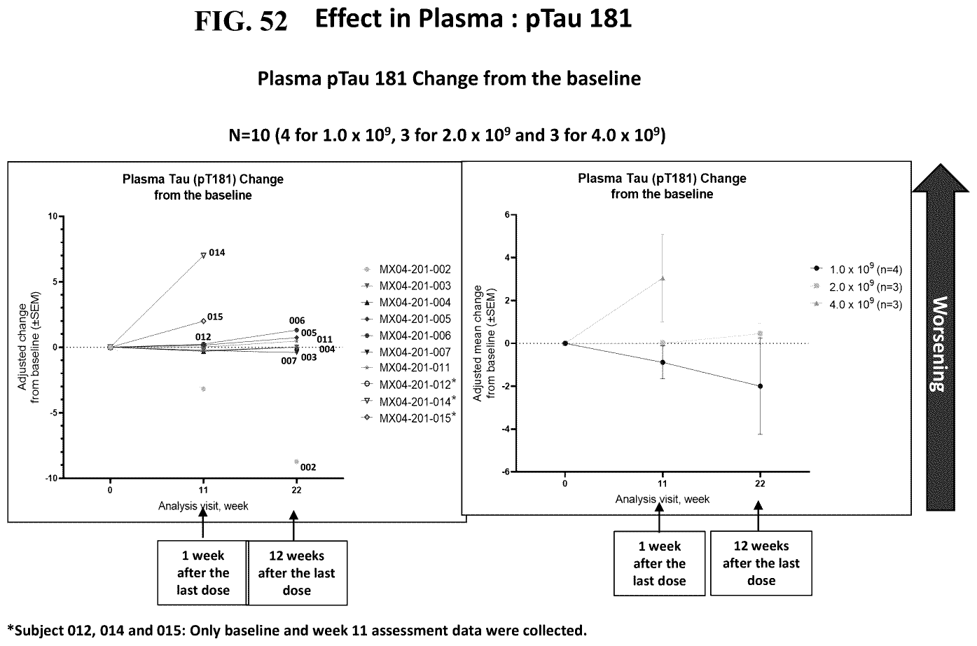

FIG. 52 shows line graphs of p-tau 181 changes in the plasma of subjects treated with SNK01. Right panel shows the mean change over time, grouped according to dosage.

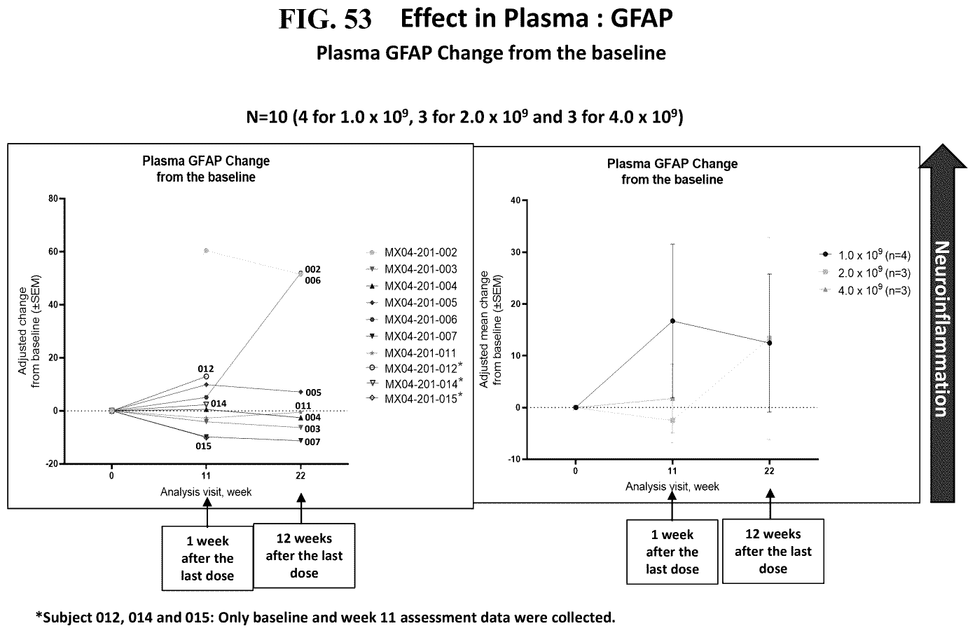

FIG. 53 shows line graphs of GFAP changes in the plasma of subjects treated with SNK01. Right panel shows the mean change over time, grouped according to dosage.

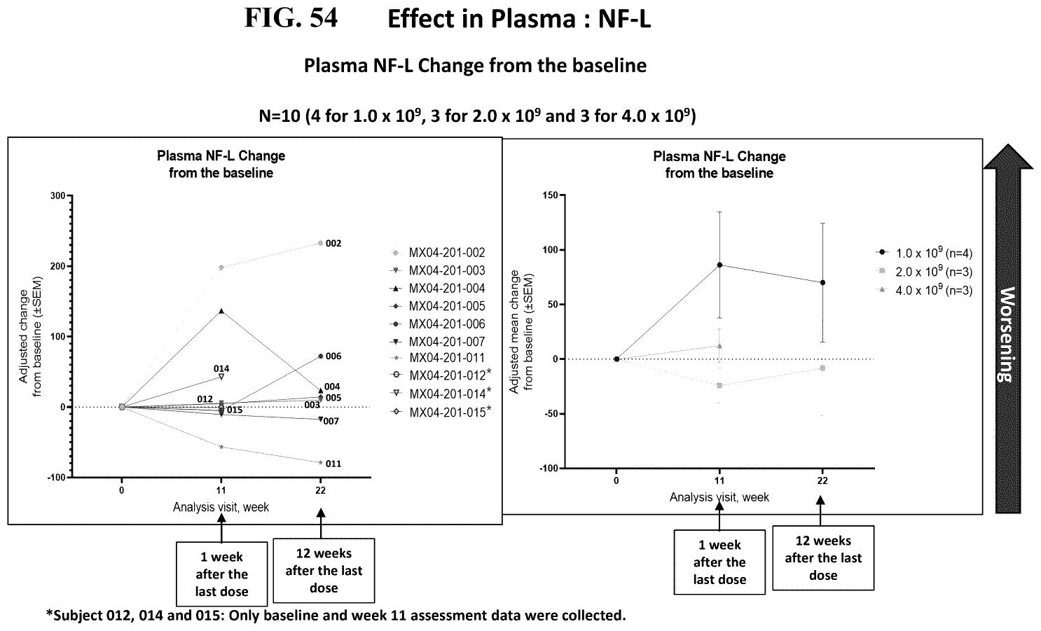

FIG. 54 shows line graphs of NfL changes in the plasma of subjects treated with SNK01. Right panel shows the mean change over time, grouped according to dosage.

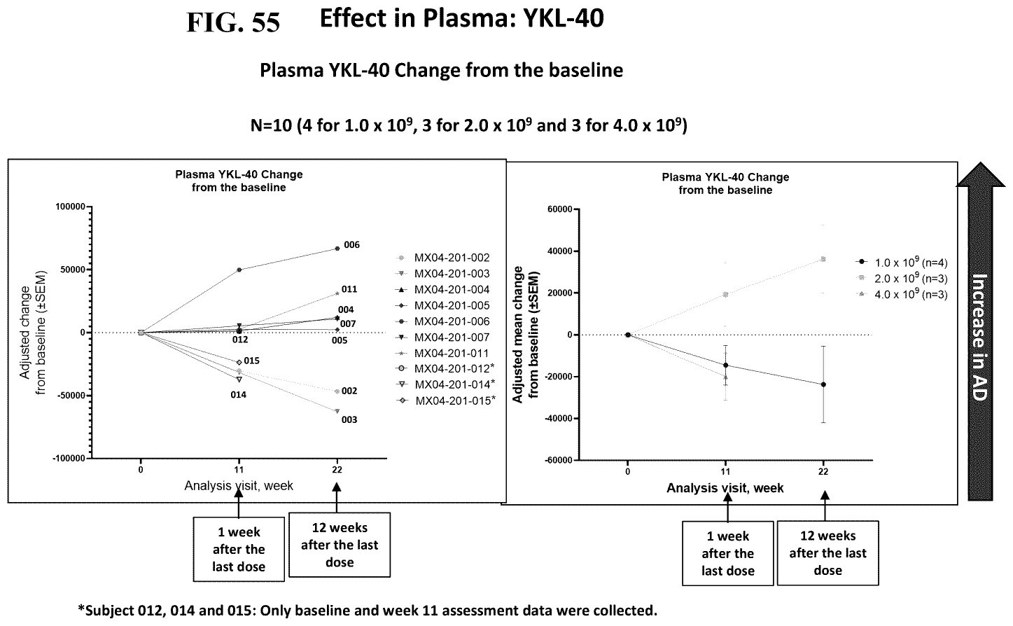

FIG. 55 shows line graphs of YKL-40 changes in the plasma of subjects treated with SNK01. Right panel shows the mean change over time, grouped according to dosage.

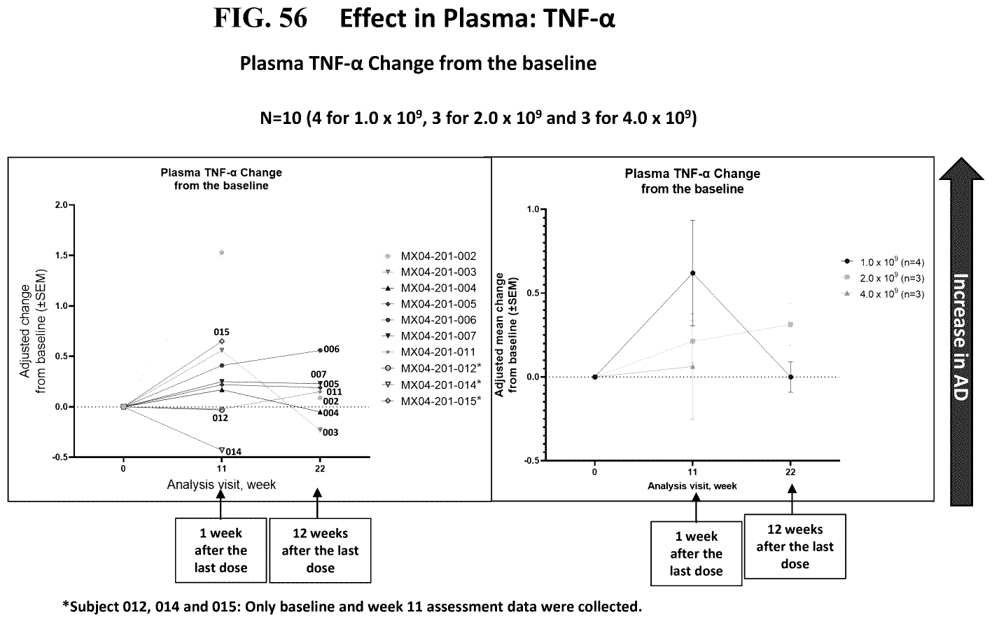

FIG. 56 shows line graphs of TNF-α changes in the plasma of subjects treated with SNK01. Right panel shows the mean change over time, grouped according to dosage.

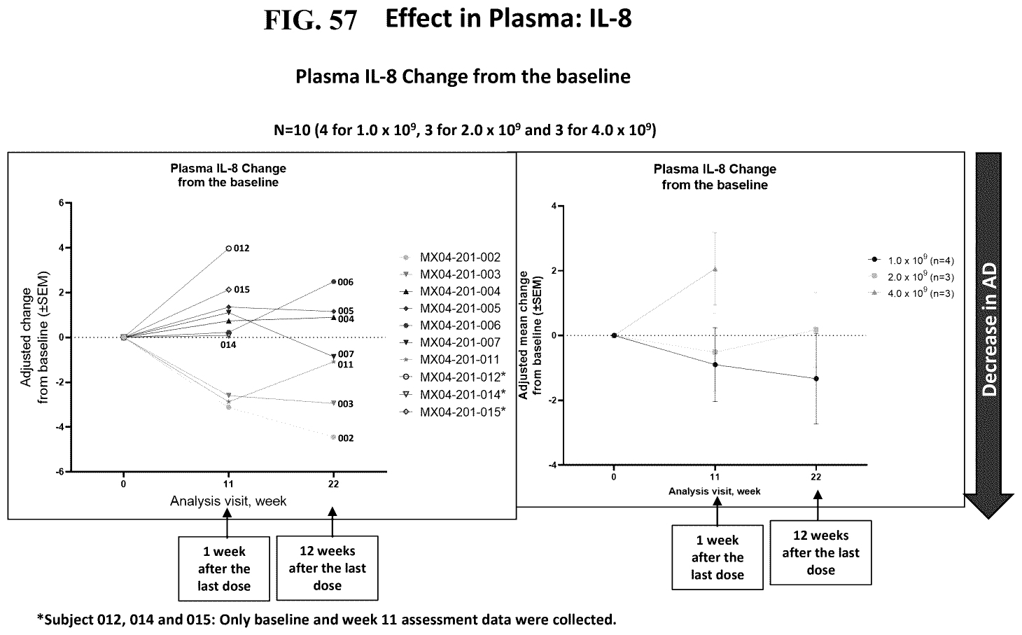

FIG. 57 shows line graphs of IL-8 changes in the plasma of subjects treated with SNK01. Right panel shows the mean change over time, grouped according to dosage.

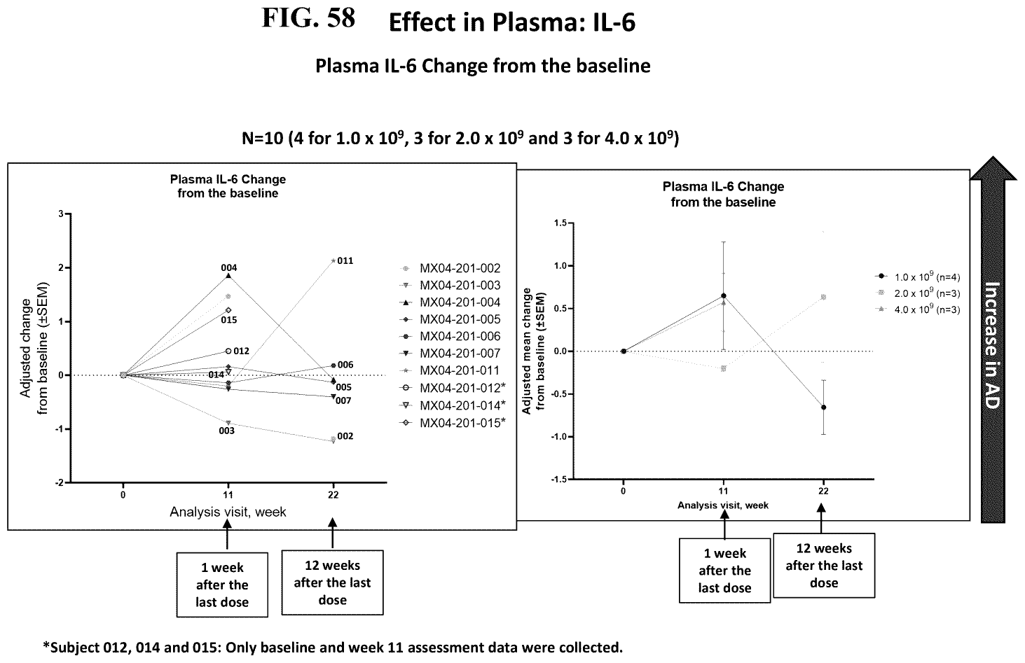

FIG. 58 shows line graphs of IL-6 changes in the plasma of subjects treated with SNK01.

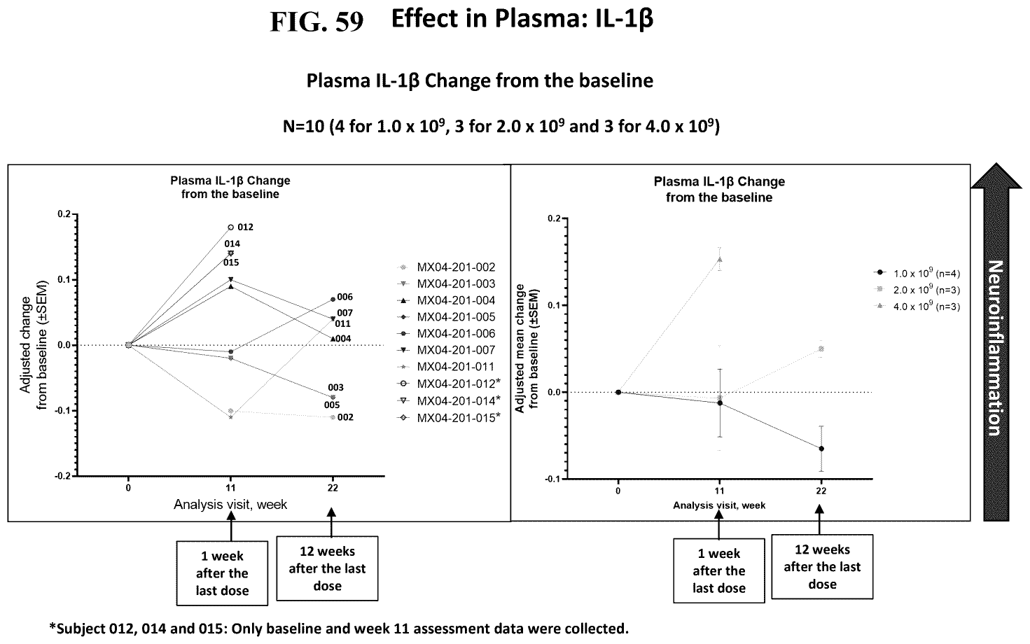

FIG. 59 shows line graphs of IL-1β changes in the plasma of subjects treated with SNK01. Right panel shows the mean change over time, grouped according to dosage.

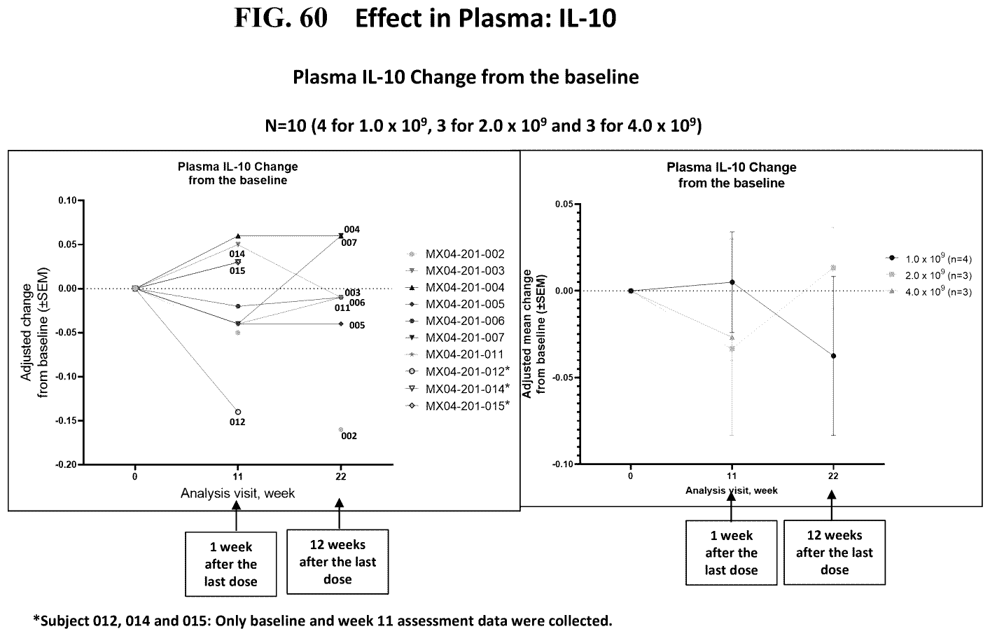

FIG. 60 shows line graphs of IL-10 changes in the plasma of subjects treated with SNK01. Right panel shows the mean change over time, grouped according to dosage.

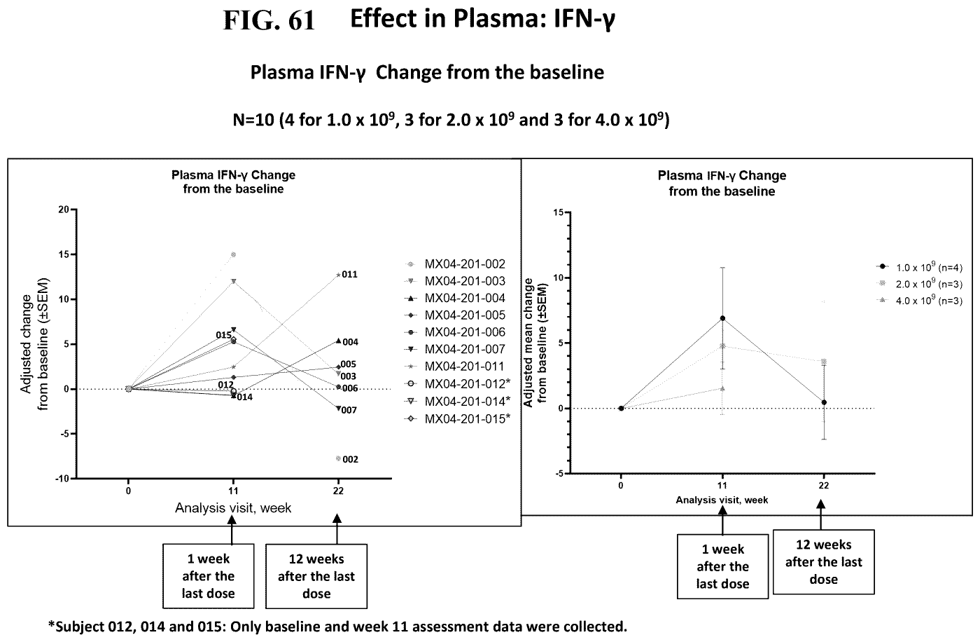

FIG. 61 shows line graphs of IFN-γ changes in the plasma of subjects treated with SNK01. Right panel shows the mean change over time, grouped according to dosage.

### Example 14 CSF Immunophenotype Markers for Alzheimer's Disease

OBJECTIVE AND METHOD Alzheimer's disease (AD) is characterized by extracellular deposits of fibrillar amyloid-beta peptides and aggregates of the phosphorylated microtubule-associated protein tau in neurofibrillary tangles. These proteins accumulate in the brain causes chronic deposition and lead to an inflammatory cascade involving alterations in the cross talks between glial cells and neurons (Yan 2021). Studies have shown T cells contribute indirectly to neuroinflammation by secreting proinflammatory mediators via direct crosstalk with glial cells and infiltrating the brain. (Dai 2020, Chen 2023). It is widely accepted that neuroinflammation in AD is driven by microglia and astrocytes while T cells are believed to be key mediators of the inflammatory response. While neuroinflammation can be a potentially beneficial defense mechanism that initially protects the brain by inhibiting diverse pathogens and clearing cellular debris, persistent inflammation can adversely affect neuronal plasticity, impair memory, and is generally considered as a main driver of tissue damage in neurodegenerative disorders (Kwon 2020). NK cells have been shown to have a protective role in other diseases caused by autoreactive T cells through cytokine production and direct killing of T cells.

Cerebrospinal fluid (CSF) from subjects with AD who participated in the autologous NK cell therapy (SNK01) were collected for the assessment of cell therapy treatment responses. Immunophenotyping of the immune cell subset frequencies and receptor expressions was done flow cytometrically. Immunophenotyping by flow cytometry was performed to analyze the expression of cell markers in a single-cell suspension from a sample of biofluid. The process identifies cells based on the types of antigens present on the cell surface or expressed intracellularly.

CSF samples were incubated with specific fluorophore-conjugated antibodies directed against the antigens of the receptor's molecules and protein molecules. The conjugated antibodies bind to the corresponding specific antigens that are presented on each single cell. After washing away the unbound antibodies, cells were then analyzed using a flow cytometer. The flow cytometer combines fluidics, optics, and electronics to convert target expressions to a measurable signal output. Briefly, the fluidics system is responsible for the acquisition and direction of cells into a stream, which enables the analysis of single cells. The optics system consists of lasers, filters, and detectors; lasers excite the fluorophores, filters direct the path of light, and detectors convert the light into an electronic signal. Lastly, the electronic component processes the output from the detector and digitizes the information for subsequent analysis using flow cytometry data analysis software to determine the quasi-quantitation of the targeted immune cell subset frequencies and receptor expressions.

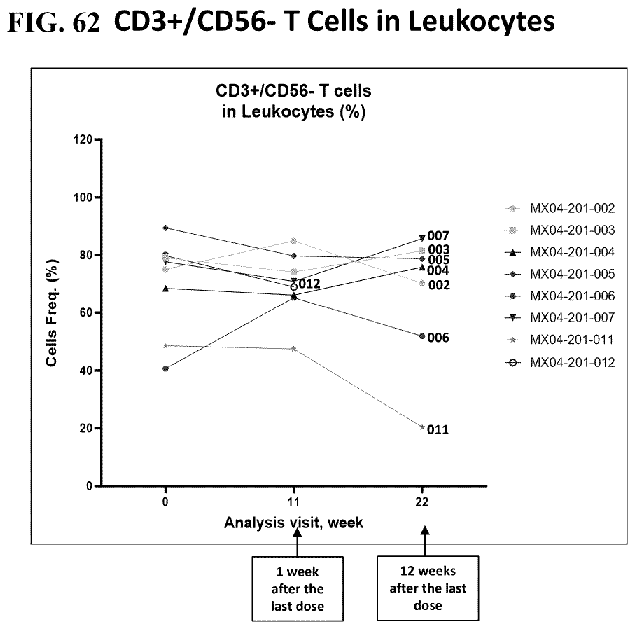

Results of the immunophenotyping are shown in FIGS. 62-73.

FIG. 62 shows a line graph of the percentage of CD3+CD56− T cells in the Leukocytes of subjects treated with NK cells.

FIG. 63 shows a line graph of the change from the baseline in the frequency of CD3+CD56− T cells in Leukocytes in subjects treated with NK cells.

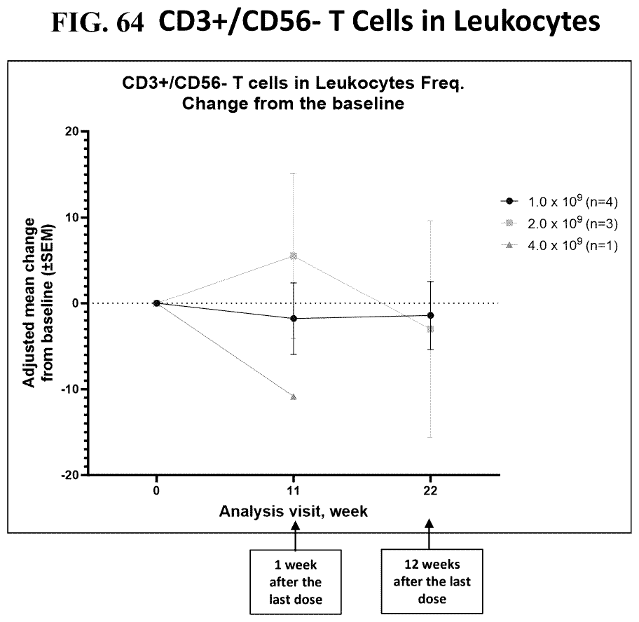

FIG. 64 shows a line graph of the mean change from baseline in the frequency of CD3+CD56− T cells in Leukocytes in subjects treated with different doses of NK cells.

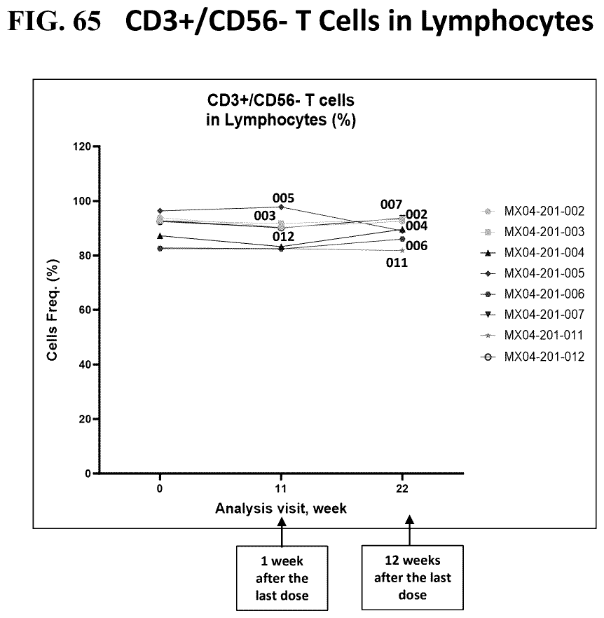

FIG. 65 shows a line graph of the percentage of CD3+CD56− T cells in Lymphocytes of subjects treated with NK cells.

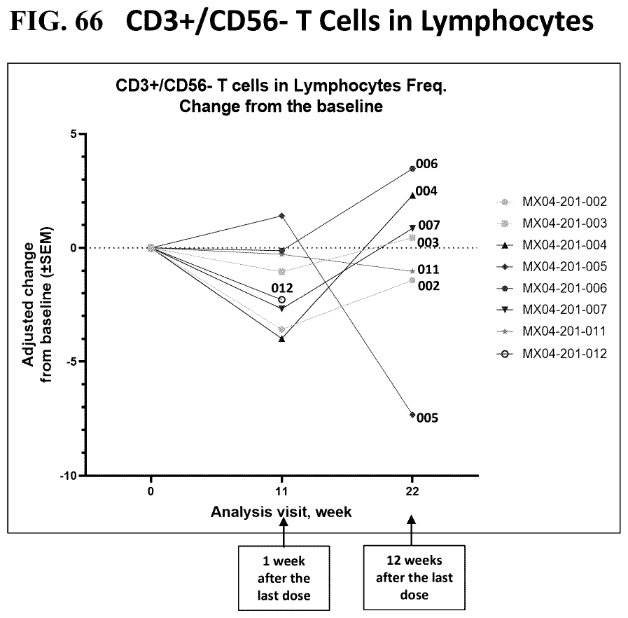

FIG. 66 shows a line of the change from the baseline in the frequency of CD3+CD56− T cells in Lymphocytes in subjects treated with NK cells.

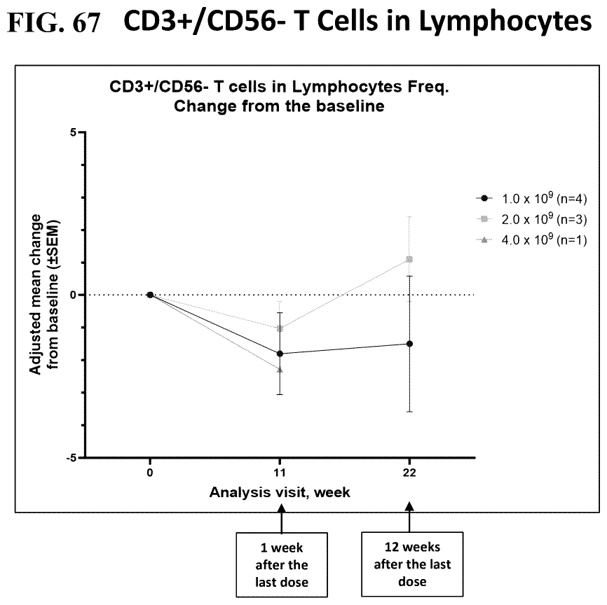

FIG. 67 shows a line graph of the mean change from baseline in the frequency of CD3+CD56− T cells in Lymphocytes in subjects treated with different doses of NK cells.

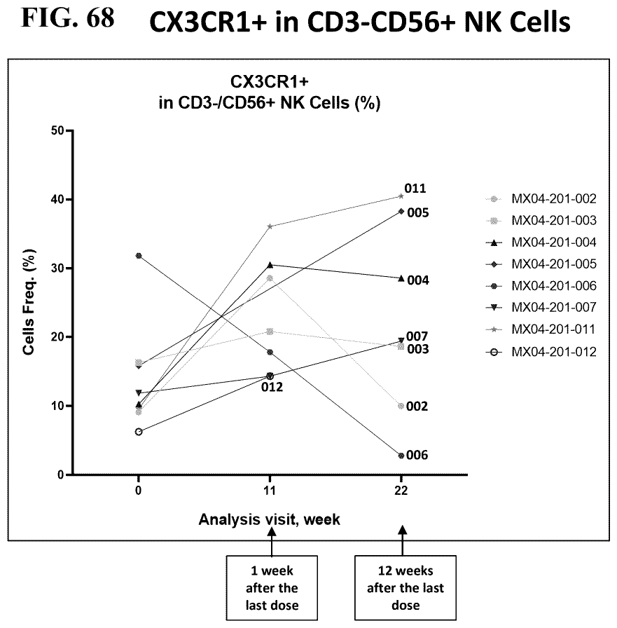

FIG. 68 shows a line graph of the percentage of CX3CR1+ cells in CD3−CD56+ NK Cells from subjects treated with NK cells.

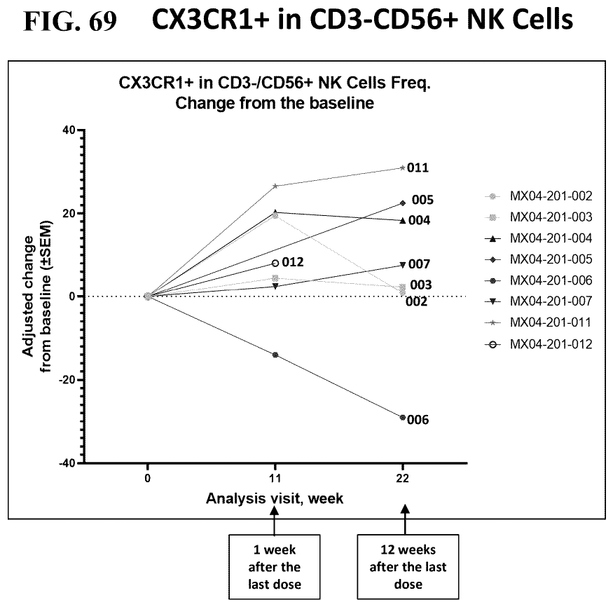

FIG. 69 shows a line graph of the change from the baseline in CX3CR1+ cells in CD3−CD56+ NK Cells in subjects treated with NK cells.

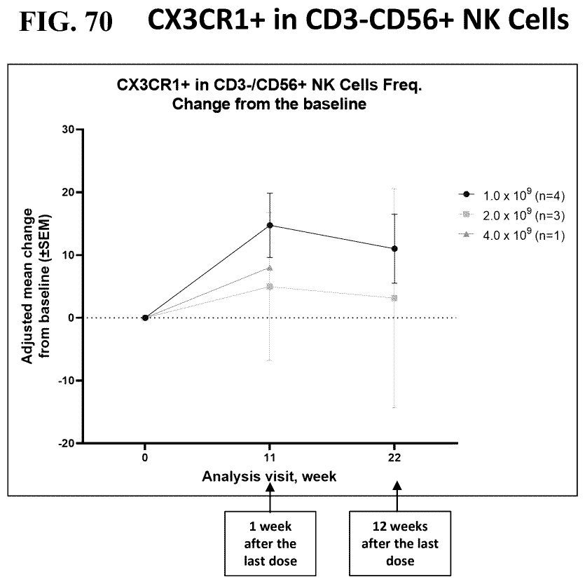

FIG. 70 shows a line graph of the mean change from baseline in the percentage of CX3CR1+ cells in CD3−CD56+ NK Cells in subjects treated with different doses of NK cells.

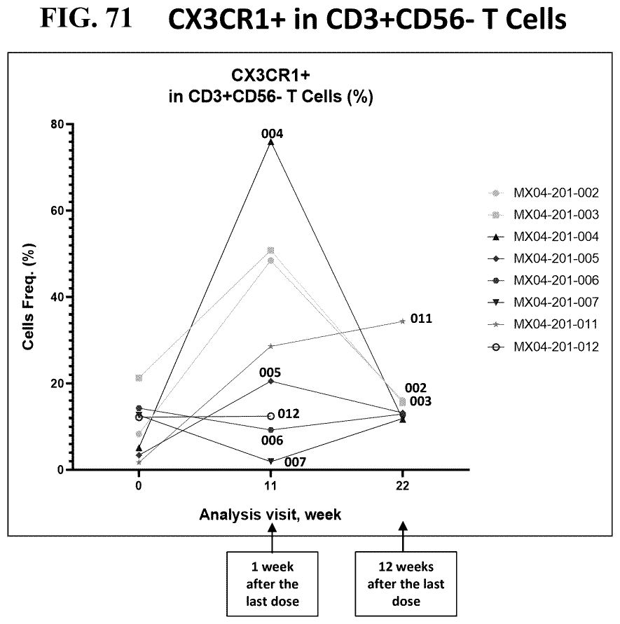

FIG. 71 shows a line graph of the percentage of CX3CR1+ cells in CD3+CD56− T Cells from subjects treated with NK cells.

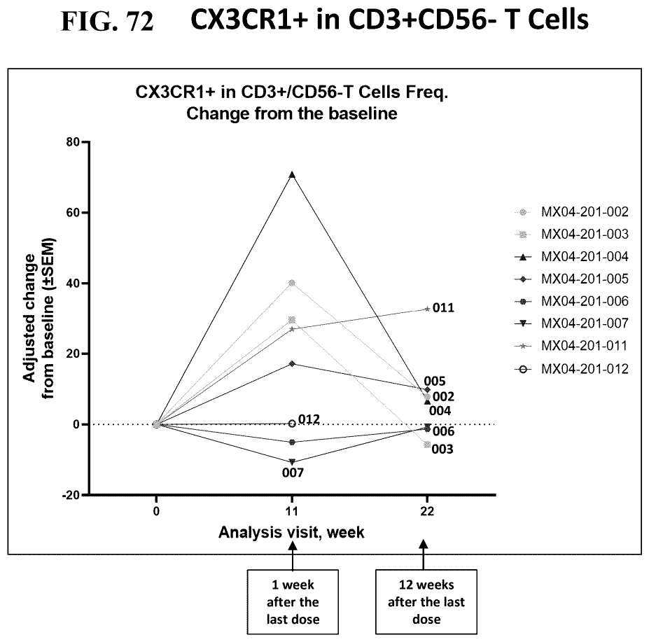

FIG. 72 shows a line graph of the change from the baseline in the percentage of CX3CR1+ cells in CD3+CD56− T Cells in subjects treated with NK cells.

FIG. 73 shows a line graph of the mean change from baseline in the percentage of CX3CR1+ cells in CD3+CD56− T Cells in subjects treated with different doses of NK cells.

### Background of the Markers

The chronic deposit and buildup of amyloid proteins has been found to illicit a pro-inflammatory dysregulation of the adaptive immune system Yan 2021) with increased T cell autoreactivity to amyloid proteins (Monsonego 2003. Mate 2015). T cells have very high CXCR3 expression and migrate to brain via CXCR3 to CXCL10 positive astrocytes that are associated with protein deposits. (Lu 201), Xia 2000). CD4+ and CD8+ T cells cause autoimmune inflammation and damage neurons in the brain (Monsonego, 2003, Male 2015, Lindestram Arelehamm 2022). NK cells can secrete interferon gamma to activate macrophages and microglia to phagocytose misfolded proteins amyloid-beta and tau tangles. (Earls 2020, Marsh 2119). SNK01 cells also traffic into the brain due to their high expression of CXCR3 and are chemoattracted by CXCL 10 positive astrocytes. SNK01 can identify and eliminate autoreactive T cells to reduce neuroinflammation. (Rabinovich 2063, Lu 2667, Nielsen 2014, Gross 2016, Schuster

In the brain, the CX3CR1 receptor is predominantly expressed in microglia. Its ligand is the secreted soluble form of fractalkine (CX3CL1) and is constitutively expressed by neurons. CX3CL1 exerts an inhibitory signal, maintaining microglia in a resting state. (Hemonnot 2019). CX3CL1 is an essential chemokine, for regulating adhesion and chemotaxis through binding to CX3CR1, which plays a critical role in the crosstalk between glial cells and neurons by direct or indirect ways in the central nervous system (CNS). CX3CL1/CX3CR1 axis regulates microglial activation and function, neuronal survival and synaptic function by controlling the release of inflammatory cytokines and synaptic plasticity in the course of neurological disease. CX3CL1/CX3CR1 is necessary for the brain to maintain the homeostasis and effectively ameliorate inflammatory response in damaged brain via regulating the balance of pro- and anti-inflammatory cytokines such as tumor necrosis factor-alpha (TNF-α), interleukin-6 (IL-6). CX3CL1/CX3CR1 binding promotes microglial activation and phagocytosis, thereby promoting the clearance of extracellular am loid beta (AP) plaque, and attenuating p-tau (Luo 2019. Subbarayan 2022)

### REFERENCES

Yan et. al, Dysregulation of the Adaptive Immune System in Patients with Early-Stage Parkinson's Disease—Neurol Neuroimmunol Neuroinflamm (2021) 8:31036
Dai L, Shen Y. Insights into T-cell dysfunction in Alzheimer's disease. Aging Cell. 2021 December; 20(12):e13511. doi: 10.1111/acel.13511. Epub 2021 Nov. 1. PMID: 34725916; PMCID: PMC8672785.
Chen, X., Firulyova, M., Manis, M. et al. Microglia-mediated T cell infiltration drives neurodegeneration in tauopathy. Nature 615, 668-677 (2023).
Kwon et. al,—Neuroinflammation in neurodegenerative disorders: the roles of microglia and astrocytes Translational Neurodegeneration (2020) 9:42
Monsonego et. al, Increased T cell reactivity to amyloid B proteins in older humans and patients with Alzheimers' disease (2003)—J. Clin. Invest 112:415
Mate Function and Redox State of Peritoneal Leukocytes as Preclinical and Prodromic Markers in a Longitudinal Study of Triple-Transgenic Mice for Alzheimer's Disease-Journal of Alzheimer's Disease 43 (2015) 213-226
Lindestram Arlehamm et. al., a-Synuclein-specific T cell reactivity is associated with preclinical and early Parkinson's disease—Nature Communication (2020) 11:1875
Earls et al., NK cells clear α-synuclein and the depletion of NK cells exacerbates synuclein pathology in a mouse model of α-synucleinopathy. PNAS (2020) vol. 117:3 1762
Marsh et al., The adaptive immune system restrains Alzheimer's disease pathogenesis by modulating microglial function PNAS I Published online Feb. 16, 20161|E1316
Rabinovich et. al., Activated, But Not Resting, T Cells Can Be Recognized and Killed by Syngeneic NK Cells J Immunol 2003; 170:3572-3576
Lu, et. al. Regulation of Activated CD4+ T Cells by NK Cells via the Qa-1-NKG2A Inhibitory Pathway Immunity (2007) 26, 593-604
Nielsen N, et. al., Cytotoxicity of CD56bright NK Cells towards Autologous Activated CD4+ T Cells Is Mediated through NKG2D, LFA-1 and TRAIL and Dampened via CD94/NKG2A. (2012) PLoS ONE 7(2): e31959.
Gross et., al., Impaired NK-mediated regulation of T-cell activity in multiple sclerosis is reconstituted by IL-2 receptor modulation PNAS (2016) e2973
Schuster I S, Coudert J D, Andoniou C E, Degli-Esposti M A. “Natural Regulators”: NK Cells as Modulators of T Cell Immunity. Front Immunol. 2016 Jun. 14; 7:235. doi: 10.3389/fimmu.2016.00235. PMID: 27379097; PMCID: PMC4905977.
Hemonnot A L, Hua J, Ulmann L, Hirbec H. Microglia in Alzheimer Disease: Well-Known Targets and New Opportunities. Front Aging Neurosci. 2019 Aug. 30; 11:233. doi: 10.3389/fnagi.2019.00233. PMID: 31543810; PMCID: PMC6730262.
Piao Luo, Shi-feng Chu, Zhao Zhang, Cong-yuan Xia, Nai-hong Chen, Fractalkine/CX3CR1 is involved in the cross-talk between neuron and glia in neurological diseases, Brain Research Bulletin, Volume 146, 2019, Pages 12-21, ISSN 0361-9230,
Meena S. Subbarayan, Aurelie Joly-Amado, Paula C. Bickford. Kevin R. Nash, CX3CL1/CX3CR1 signaling targets for the treatment of neurodegenerative diseases, Pharmacology & Therapeutics, Volume 231, 2022, 107989, ISSN 0163-7258,

The foregoing description of the exemplary embodiments has been presented only for the purposes of illustration and description and is not intended to be exhaustive or to limit the invention to the precise forms disclosed. Many modifications and variations are possible in light of the above teaching. It is contemplated that various combinations or sub combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention is defined by the appended claims rather than the foregoing description and the exemplary embodiments described therein.

What is claimed is:

1. A method of treating Alzheimer's disease in a subject, the method comprising:
a. identifying a subject, wherein the subject has Alzheimer's; and
b. administering to the subject an expanded natural killer (NK) cell population, wherein the NK cells are expanded by a method comprising:
i) isolating at least one of CD56+ cells and/or CD3−/CD56+ cells from the PBMCs;
ii) co-culturing the at least one of CD56+ cells and/or CD3−/CD56+ cells with a combination of feeder cells in the presence of at least two cytokines;
iii) wherein the combination of feeder cells comprises irradiated Jurkat cells and irradiated Epstein-Barr virus transformed lymphocyte continuous line (EBV-LCL) cells; and
iv) wherein the at least two cytokines comprise IL-2 and IL-21.

2. The method of claim 1, wherein the amount of expanded NK cells administered to a subject is a therapeutically effective amount.

3. The method of claim 2, wherein the therapeutically effective amount of expanded NK cells comprises 0.1×10⁹ to 9×10⁹ cells.

4. The method of claim 1, wherein at least 1, 2, 3, 4, 5, 6, 7, 8, 9, 10, 11, 12, 13, 14, 15, 16, 17, 18, 19, 20 or more doses of expanded NK cells is administered to the subject.

5. The method of claim 1, wherein IL-2 is added at a concentration of 50-1000 IU/mL during step ii).

6. The method of claim 1, wherein IL-21 is added at a concentration of 10-100 ng/mL during step ii).

7. The method of claim 1, wherein the Mini-Mental State Exam (MMSE) score of the subject is between 24-30, 19-23, or 10-18 after treatment with expanded NK cells.

8. A method of cell therapy comprising:
a. identifying a subject, wherein the subject has Alzheimer's disease; and
b. administering to the subject an expanded NK cell population, wherein the NK cells are expanded by a method comprising:
i) isolating at least one of CD56+ cells and/or CD3−/CD56+ cells from the PBMCs;
ii) co-culturing the at least one of CD56+ cells and/or CD3−/CD56+ cells with a combination of feeder cells in the presence of at least two cytokines;
iii) wherein the combination of feeder cells comprises irradiated Jurkat cells and irradiated Epstein-Barr virus transformed lymphocyte continuous line (EBV-LCL) cells; and
iv) wherein the at least two cytokines comprise IL-2 and IL-21.

9. A population of expanded NK cells, wherein the NK cells were expanded by a method that comprises:
v) isolating at least one of CD56+ cells and/or CD3−/CD56+ cells from the PBMCs;
vi) co-culturing the at least one of CD56+ cells and/or CD3−/CD56+ cells with a combination of feeder cells in the presence of at least two cytokines;
vii) wherein the combination of feeder cells comprises irradiated Jurkat cells and irradiated Epstein-Barr virus transformed lymphocyte continuous line (EBV-LCL) cells; and
viii) wherein the at least two cytokines comprise IL-2 and IL-21; and
wherein the population of expanded NK cells has been administered to a subject who has Alzheimer's disease.

10. The population of cells of claim 9, wherein the amount of expanded NK cells administered to a subject is a therapeutically effective amount.

11. The method of claim 10, wherein the therapeutically effective amount of expanded NK cells comprises 0.1×10⁹ to 9×10⁹ cells.

12. The population of cells of claim 9, wherein at least 1, 2, 3, 4, 5, 6, 7, 8, 9, 10, 11, 12, 13, 14, 15, 16, 17, 18, 19, 20 or more doses of expanded NK cells is administered to the subject.

13. The population of cells of claim 9, wherein IL-2 is added at a concentration of 50-1000 IU/mL during step ii).

14. The population of cells of claim 9, wherein IL-21 is added at a concentration of 10-100 ng/mL during step ii).

15. The population of cells of claim 9, wherein the Mini-Mental State Exam (MMSE) score of the subject is between 24-30, 19-23, or 10-18 after treatment with expanded NK cells.

16. A method of treating Alzheimer's disease in a subject, the method comprising:
a. identifying a subject, wherein the subject has Alzheimer's disease; and
b. administering to the subject a therapeutically effective amount of a NK cell population (e.g., an autologous NK cell population).

17. The method of any one of the preceding claims, further comprising administration of one or more secondary Alzheimer's disease therapeutics.

18. The method of claim 17, wherein the one or more secondary Alzheimer's disease therapeutics comprises aducanumab.

19. The method of claim 17, wherein the NK cells and the one or more secondary Alzheimer's disease therapeutics are co-administered.

20. The method of claim 17, wherein the NK cells and the one or more secondary Alzheimer's disease therapeutics are administered for 1, 2, 3, 4, 5, 6, 7, 8, 9, 10, 11, 12, 14, 16, 20, 24, 28, 32, or 36 weeks.

21. The method of claim 17, wherein the NK cells and the one or more secondary Alzheimer's disease therapeutics are alternately administered for 1, 2, 3, 4, 5, 6, 7, 8, 9, 10, 11, 12, 14, 16, 20, 24, 28, 32, or 36 weeks.

22. The method of claim 17, wherein administration of the NK cells in combination with one or more secondary Alzheimer's therapies and/or therapeutics, reduces the time required for the one or more secondary Alzheimer's disease therapeutics to achieve a therapeutic effect by 1-fold, 2-fold, 3-fold, 4-fold, 5-fold, 6-fold, 7-fold, 8-fold, 9-fold, or 10-fold.

23. The method of claim 17, wherein administration of the NK cells in combination with one or more secondary Alzheimer's therapies and/or therapeutics, reduces the time required for the NK cells to achieve a therapeutic effect by 1-fold, 2-fold, 3-fold, 4-fold, 5-fold, 6-fold, 7-fold, 8-fold, 9-fold, or 10-fold.

24. The method of claim 17, wherein administration of the NK cells in combination with one or more secondary Alzheimer's therapies and/or therapeutics, reduces the number of doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.

25. The method of claim 17, wherein administration of the NK cells in combination with one or more secondary Alzheimer's therapies and/or therapeutics, reduces the number of doses of the NK cells required to achieve a therapeutic effect by 1-fold, 2-fold, 3-fold, 4-fold, 5-fold, 6-fold, 7-fold, 8-fold, 9-fold, or 10-fold.

26. The method of claim 17, wherein administration of the NK cells in combination with one or more secondary Alzheimer's therapies and/or therapeutics, reduces the doses of the one or more secondary Alzheimer's disease therapeutics required to achieve a therapeutic effect by 1-fold, 2-fold, 3-fold, 4-fold, 5-fold, 6-fold, 7-fold, 8-fold, 9-fold, or 10-fold.

27. The method of claim 17, wherein administration of the NK cells in combination with one or more secondary Alzheimer's therapies and/or therapeutics, reduces the doses of the NK cells required to achieve a therapeutic effect by 1-fold, 2-fold, 3-fold, 4-fold, 5-fold, 6-fold, 7-fold, 8-fold, 9-fold, or 10-fold.

28. A kit comprising the NK cell population of any one of the preceding claims and one or more secondary Alzheimer's disease therapeutics.

29. A formulation comprising the NK cell population of any one of the preceding claims and one or more secondary Alzheimer's disease therapeutics.

30. The method of any one of the preceding claims, wherein identifying a subject as having Alzheimer's disease comprises detecting and/or quantifying one or more biomarkers.

31. The method of claim 30, wherein the one or more biomarkers are quantified and/or detected in the subject's cerebrospinal fluid and/or plasma from peripheral blood.

32. The method of claim 30, wherein the one or more biomarkers comprise YKL-40, CX3CL1, TNF-α, IL-6, IL-8, IL-12/IL-23p40, and/or sTREM2, or any combination thereof.

33. The method of claim 30, wherein the one or more biomarkers comprise Aβ-42/40, Aβ-42, total Tau, pTau, GFAP, and/or NfL, or any combination thereof.

34. The method of any one of the preceding claims, wherein identifying a subject as having Alzheimer's disease comprises administering one or more cognitive assessments.

35. The method of claim 34, wherein the one or more cognitive assessments comprises a Clinical Dementia Rating, Alzheimer's disease assessment scale-cognitive subscale, mini-mental status exam, or any combination thereof.

36. The method of any one of the preceding claims, wherein administration of the NK cells decreases neuroinflammation in the subject as compared to the level of neuroinflammation in the subject prior to administration of the NK cells.

37. The method of any one of the preceding claims, wherein the NK cells are administered intravenously.

38. The method of any one of the preceding claims, wherein the expanded NK cell population or the NK cell population is or comprises SNK01.
